(19) United States
(12) Patent Application Publication
(10) Pub. No.: US 2024/0180842 A1
(43) Pub. Date: Jun. 6, 2024

(54) COLON TARGETED DRUG DELIVERY

(71) Applicant: Axial Therapeutics, Inc., Woburn, WA (US)

(72) Inventors: Hitesh BHAGAT, Framingham, MA (US); Olivia R. McCarthy, Quincy, MA (US); Rajesh R. Wakaskar, Woburn, WA (US)

(21) Appl. No.: 18/525,072

(22) Filed: Nov. 30, 2023

Related U.S. Application Data
(63) Continuation of application No. 18/477,387, filed on Sep. 28, 2023.
Application No. 18/477,387 is a continuation of application No. PCT/US2023/012999, filed on Feb. 14, 2023.
(60) Provisional application No. 63/310,095, filed on Feb. 14, 2022.

(51) Int. Cl. A61K 9/48; A61K 9/00

(52) U.S. Cl. CPC A61K 9/485; A61K 9/0056; A61K 9/4816; A61K 9/4825

(57) ABSTRACT

The present disclosure relates to a pharmaceutical preparation for oral delivery of a therapeutic agent to the colon.

### CROSS-REFERENCE TO RELATED APPLICATIONS

This application is a continuation of U.S. patent application Ser. No. 18/477,387, filed Sep. 28, 2023, which is a continuation of International Patent Application No. PCT/US2023/012999, filed Feb. 14, 2023, which claims priority to, and the benefit of, U.S. Provisional Patent Application No. 63/310,095, filed Feb. 14, 2022, the entire contents of each of which is incorporated herein by reference in their entireties.

### TECHNICAL FIELD

The present disclosure relates to a pharmaceutical preparation for oral delivery of a therapeutic agent to the colon.

### BACKGROUND

Non-absorbable, non-digestible, biocompatible polymers have been used for lowering cholesterol and systemic phosphate levels by targeting adsorption of cholesterol and free phosphate in the gut. These products are biocompatible ion exchange resins that are not absorbed to any significant extent and are excreted from the gastrointestinal (GI) tract after binding their target molecules. For example, the ion exchange resin cholestyramine has been used for sequestering bile acids, which are cholesterol derivatives, so as to lower cholesterol.

Additionally, non-absorbable, non-digestible, biocompatible activated carbon preparations have been investigated to counteract the effects of toxins in poisoning and drug overdoses (e.g., Dillon et al. (1989), Ann. Emerg. Med. 18(5):547-52; Kieslichova et al. (2018), Transplantation Proc. 50:192-197), and uremic toxins in the treatment of chronic kidney disease (CKD) (e.g., Schulman et al. (2016), BMC Nephrology 17:141). For example, an activated carbon particle preparation has been developed and utilized for delaying dialysis in subjects suffering from chronic kidney disease, but the clinical utility of this approach has not been widely accepted, multiple meta-analyses have indicated no clear clinical benefit, and a later stage clinical trial in the US failed to prove efficacy.

Furthermore, it has reported that increased levels of bacterial metabolites, such as p-cresyl sulfate (pCs or PCS), 4-ethyl-phenyl sulfate (4-EPS), and 3-indoxyl sulfate (IS), have been detected in subjects with autism spectrum disorder (ASD). Recently, treatments using oral adsorbents to lower the levels of bacterial metabolites in the gut have been developed by Axial Therapeutics for the treatment of ASD populations.

Previously, oral adsorbents have predominantly been administered in immediate release form as a powder for dispersion, or as capsules or tablets.

Further, the current state of the art is directed toward targeting the terminal ileum/caecum for release by colon-targeted drug delivery systems, and not the descending colon.

Spherical carbon adsorbent has been prescribed to pre-dialysis CKD subjects who are diagnosed with progressive CKD in order to improve uremic symptoms and delay dialysis initiation. It is known that uremic symptoms such as anorexia, bad breath, and nausea caused by the accumulation of uremic toxins in the body due to impaired renal functions are improved by taking spherical carbon adsorbent (Keizo Koide et al. (1987), Rinsho Hyoka (Clinical Evaluation) 15(3):527-564). In addition, it is known that some uremic toxins, such as p-cresyl sulfate originating from p-cresol produced by intestinal bacteria, actively deteriorate renal functions, and adsorption of such uremic toxins in the lumen of the gastro-intestinal (GI) tract reduces the amount of uremic toxins absorbed into the body, which makes it possible to suppress progression of renal dysfunction (decrease in eGFR and increase in serum creatinine level and BUN) of pre-dialysis CKD subjects and to delay transition to dialysis (Tadao Akizawa et al. (1998), Kidney and Dialysis, 45(3):373-388). It is expected that reduction of uremic toxins improves vascular endothelial functions and suppresses calcification (Ayinuer Adijiang et al. (2008), Nephrol Dial Transplant 23:1892-1901), and moreover it is expected that increasing the response of CERA (Continuous EPO Receptor Activator) can contribute to treating or preventing anemia (I-Wen Wu et al. (2014), Nephrol Dial Transplant 29(9):1719-1727).

In Japan, capsule preparations and fine granule preparations of spherical carbon adsorbent having brand names Kremezin® Capsule 200 mg and “Kremezin® Fine Granules 2 g are commercially available. Attempts have been made to form tablet preparations in order to reduce the volume of Kremezin® (see WO2012/121202), but practical use is not achieved yet.

### SUMMARY

The present invention depends, in part, on the development of improved pharmaceutical compositions for delivery of therapeutic agents to the colon. These compositions comprise cores comprising or consisting of one or more therapeutic agents, surrounded by one or more layers which control the release of the therapeutic agent(s) to specifically target delivery to the colon, particularly the descending colon. These pharmaceutical compositions have many uses, including but not limited to (1) targeted delivery of therapeutic agents comprising orally-administered sequestrants or adsorbents for removal of intestinal metabolites (e.g., bacterial metabolites) from the colon, (2) delivery of the therapeutic agent(s) specifically to the colon where the therapeutic agent(s) can modulate the function of the colon and/or the metabolism, growth and/or survival of bacterial strains within the colonic microbiome, and (3) amelioration of drug-drug interactions by delivery of at least one therapeutic agent to the colon while another therapeutic agent is delivered to the stomach, small intestine or caecum.

Without being limited to any particular applications, therapeutic agents, or mechanisms of action, the following embodiments of the pharmaceutical compositions of the invention are described:
(1) A pharmaceutical composition for oral administration comprising a therapeutic agent core, and one or more layers for exposing a surface of the therapeutic agent core. In some embodiments, the surface of the therapeutic agent core is exposed for the first time in the colon. In other embodiments, the surface of the therapeutic agent core is exposed for the first time in the distal portion of the ileum.
(2) The pharmaceutical composition of preparation (1), wherein said therapeutic agent core comprises one or more porous materials.
(3) The pharmaceutical composition of preparation (2), wherein said porous material comprises activated carbon particles.
(4) The pharmaceutical composition of preparation (1), wherein said therapeutic agent core comprises one or more semi-synthetic glucosamine-based cationic polymers (e.g., a chitosan derivative, or Polymers A-D described herein).
(5) The pharmaceutical composition of preparation (1), wherein said therapeutic agent core comprises one polycationic resins and/or acrylamide-based polymers with cyclodextrin or other glycosyl substitutions of a plurality of hydrophobic monomers, including such resins or polymers formed into “plastic antibodies.”
(6) The pharmaceutical composition of any one of (1) to (5), wherein one or more of said plurality of layers comprise a pH-sensitive layer.
(7) The pharmaceutical composition of any one of (1) to (6), wherein said plurality of layers comprise a hydrolyzed zein-derived material.
(8) The pharmaceutical composition of any one of (1) to (7), wherein said plurality of layers further comprise a polysaccharide.
(9) The pharmaceutical composition of (7), wherein the hydrolyzed zein-derived material is applied as the first outer layer over the first, the second, or the third inner layer further comprising a swelling agent.
(10) The pharmaceutical composition of (8), wherein said plurality of layers further comprising a polysaccharide is applied as the first outer layer over a first, second, or third inner layer further comprising a swelling agent.
(11) The pharmaceutical composition of any one of (4) to (10), wherein said first outer layer comprises a water-insoluble polymer.
(12) The pharmaceutical composition of any one of (5) to (11), wherein the first, the second, or the third inner layers comprise a “sticky polymer” applied onto the therapeutic agent core surface.
(13) A pharmaceutical composition of any one of (5) to (12), wherein said first outer layer comprises a pH-responsive enteric polymer and one or more of the first, the second or the third inner layers comprise a protein or hydrolyzed zein-derived material.
(14) A pharmaceutical composition of any one of (5) to (13), wherein said first outer layer comprises a pH-responsive enteric polymer; the first or the second inner layer comprise a layer of protein or hydrolyzed zein-derived material; and the third inner layer comprises a swelling agent.
(15) Additionally, in any of the foregoing embodiments, the enteric polymer can be one or more selected from methacrylic acid copolymer L, methacrylic acid copolymer S, methacrylic acid copolymer LD, hydroxypropyl methylcellulose acetate succinate, cellulose acetate phthalate, and cellulose acetate succinate.
(16) In some embodiments, a pharmaceutical composition according to any one of (1) to (15), wherein the pharmaceutical composition wherein the means for exposing the therapeutic agent core surface for the first time in a delayed-burst release is a granule preparation, a capsule preparation, or a tablet preparation comprising a therapeutic agent coated with an enteric polymer. In some embodiments, the delayed-burst release is in the colon.

In some embodiments, a pharmaceutical composition according to any one listed herein, for reducing the level of a bacterial metabolite, reducing, ameliorating or treating a symptom of a neurological disorder such as autism or autism spectrum disorder (ASD); reducing the level of a blood uremic toxin or bacterial metabolite, improving a uremic symptom, delaying dialysis initiation, or protecting a renal function in a subject with CKD.

In some embodiments, a method for producing a pharmaceutical composition for oral administration according to any one listed herein, the method comprising any of the following steps:
a) a step of coating a therapeutic agent with a protein layer; a protein and polysaccharide layer; a protein and polysaccharide layer, further coated with an enteric polymer layer; a protein and polysaccharide layer which is further deposited upon a swelling polymer layer; or, above, a top layer coated with an enteric polymer layer;
b) a step of encapsulating a therapeutic agent within a capsule coated with any of the layer combinations described herein in a); and
c) a step of coating a compression-molded product of a therapeutic agent with any of the combination described herein in a).

Additionally, the present disclosure further provides those features set forth as described below:
(17) A pharmaceutical composition for oral administration, wherein a means for exposing a surface of a therapeutic agent core for the first time in a delayed-burst release in the colon is provided on said therapeutic agent.
(18) A pharmaceutical composition according to (17), wherein the means for exposing the therapeutic agent core surface for the first time in the descending colon comprises the layer of the therapeutic agent with an enteric polymer layer.
(19) A pharmaceutical composition according to (17), wherein the means for exposing the therapeutic agent core surface for the first time in the descending colon is encapsulation of the therapeutic agent, that can be coated with an enteric polymer layer, within an enteric capsule.
(20) A pharmaceutical composition according to (18) or (19), wherein the enteric polymer is a cellulose-based, vinyl-based, or acrylic-based polymer.
(21) A pharmaceutical composition according to (18) or (19), wherein the enteric polymer is an acrylic-based polymer.
(22) A pharmaceutical composition according to (18) or (19), wherein the enteric polymer is an acrylic-based polymer, and a coating of the acrylic-based polymer is 10% to 60% by weight based on the therapeutic.
(23) A pharmaceutical composition according to (18) or (19), wherein the enteric polymer is an acrylic-based polymer, and a coating of the acrylic-based polymer is 20% to 40% by weight based on the therapeutic.
(24) A pharmaceutical composition according to any one of (21) to (23), wherein the acrylic-based polymer is one or more selected from a copolymer of methacrylic acid and ethyl acrylate, a copolymer of methacrylic acid and methyl methacrylate, and a copolymer of methacrylic acid, methyl acrylate, and methyl methacrylate.
(25) A pharmaceutical composition according to any one of (21) to (23), wherein the enteric polymer is one or more selected from methacrylic acid copolymer L and methacrylic acid copolymer S.
(26) A pharmaceutical composition according to any one of (21) to (25), further comprising one or more polysaccharides selected from chitosan, resistant starches, alginate, alginates, amylose, amyloses, galactomannans, arabinoxyan, inulin, inulins, pectins, guar gums, xanthan gums, chondroitin sulfate, dextrans, cyclodextrins, Nutriose® brand soluble fiber, locust bean gums, soy polysaccharides, pea polysaccharides, and the like, among others.
(27) A pharmaceutical composition according to any one of (21) to (26), further comprising one or more swelling agents selected from cellulose derivatives including HPMC, MC; hydrocolloids including alginate, chitosan, pectin, poly (ethylene oxide), carbopol, poly (vinyl alcohol), crosslinked sodium carboxymethylcellulose, crosslinked poly (vinyl pyrrolidone), sodium starch glycolate, acrylamide, acrylic acid, 2-hydroxyethyl methacrylate, and the like, among others.
(28) A pharmaceutical composition according to any one of (21) to (27), further comprising one or more proteins selected from gelatins, albumins, soy proteins, pea proteins, maize proteins (such as zein), collagens, and the like, among others. In certain embodiments, these proteins can be partially-digested or partially-hydrolyzed.
(29) A pharmaceutical composition according to any one of (17) to (28), wherein the therapeutic agent is a spherical carbon adsorbent.
(30) A pharmaceutical composition according to any one of (17) to (29), wherein the therapeutic agent is activated carbon particles.
(31) A pharmaceutical composition according to any one of (17) to (30), for improving irritability symptom in a subject with ASD.
(32) A pharmaceutical composition according to any one of (17) to (31), for simultaneous administration, or co-administration, with a further therapeutic agent.
(33) A pharmaceutical composition according to any one of claims (17) to (32), further comprising a further therapeutic agent.
(36) A method for producing a pharmaceutical composition for oral administration, wherein a means for exposing a surface of a therapeutic agent for the first time in the colon is provided on said therapeutic agent, the method comprising a step of coating the therapeutic agent with one or more of: a protein layer; a protein and polysaccharide layer; a swelling agent layer; and enteric polymer layer.
(37) The method of (36) for producing a pharmaceutical composition for oral administration wherein a means for exposing a surface of a therapeutic agent for the first time in the colon is provided on said therapeutic agent, the method further comprising a step of encapsulating the therapeutic agent that can be coated with an enteric polymer within an enteric capsule; a step of coating a therapeutic agent with a protein layer; a protein and polysaccharide layer; a protein and polysaccharide layer, further coated with an enteric polymer layer; a protein and polysaccharide layer which is further deposited upon a swelling polymer layer; or, above, a top layer coated with an enteric polymer layer; and a step of encapsulating a therapeutic agent within a capsule coated with any of the layer combinations described herein.

The pharmaceutical preparation for oral administration of a therapeutic agent of the present disclosure has excellent or improved simultaneous usability with other therapeutic agents, thus contributing to excellent or improved medication or therapeutic agent use compliance, and is useful for: reducing the level of a bacterial metabolite, reducing, ameliorating or treating a symptom of a neurological disorder such as autism or autism spectrum disorder (ASD); reducing blood uremic toxins or bacterial metabolites, improving uremic symptoms, delaying dialysis initiation, protecting renal functions, or the like in a subject with CKD.
(38) In one preferred embodiment, the pharmaceutical composition for oral administration comprises a therapeutic agent core comprising porous activated carbon particles having substantially spherical particles that have a minimum average particle diameter of at least 0.005 mm and a maximum average particle diameter of less than 1.5 mm; and a plurality of degradable layers for exposing a surface of the therapeutic agent core for the first time in a timed-burst release in the colon, wherein there is an inner degradable layer that is a microbial responsive layer and an outer degradable layer that comprises one or more pH-responsive polymers that do not degrade in the upper gastrointestinal tract.
(39) In a further preferred embodiment, the pharmaceutical composition of (38), has an outer layer that comprises an acrylic based polymer.
(40) In a further preferred embodiment, the pharmaceutical composition of (39), the acrylic based polymer is selected from the group consisting of (a) methacrylic acid copolymer S or (b) a co-polymer of methacrylic acid and methyl methacrylate.
(41) In a further preferred embodiment, in the pharmaceutical composition of (38), (39) or (40), the outer layer comprises between about 10-60% weight of the acrylic polymer based on the therapeutic core, more preferably between 20-40% weight of the acrylic polymer based on the therapeutic core.
(42) In a further preferred embodiment, in the pharmaceutical composition of (38), (39), (40) or (41), the inner layer comprises hydroxypropylmethylcellulose.
(43) In a further preferred embodiment, in the pharmaceutical composition of (38), (39), (40) or (41), the inner layer comprises hydroxypropylmethylcellulose and a microbial responsive component selected from the group consisting of high amylose corn starch, pea protein, soluble fiber, sodium octenyl succinate starch and fructo-oligosaccharides.
(44) In a further preferred embodiment, in the pharmaceutical composition of (38), (39), (40) or (41), the inner layer comprises a polymer selected from the group consisting of (a) ethyl cellulose or (b) poly(ethyl acrylate-co-methyl methacrylate) co-polymer.
(45) In a further preferred embodiment, in the pharmaceutical composition of (38), (39), (40), (41) or (42), the inner layer comprises a polymer selected from the group consisting of (a) ethyl cellulose or (b) poly(ethyl acrylate-co-methyl methacrylate) co-polymer and a microbial responsive component selected from the group consisting of high amylose corn starch, pea protein, soluble fiber, sodium octenyl succinate starch and fructo-oligosaccharides.
(46) In a further preferred embodiment, the pharmaceutical composition of (42) or (45) has a microbial responsive component that is pea protein.
(47) In a further preferred embodiment, the pharmaceutical composition of (38), (39), (40), (41), (42), (43), (44), (45), or (46), having an inner layer and/or the outer layer that additionally comprises an anti-tacking agent.
(48) In a further preferred embodiment, the pharmaceutical composition of (38), (39), (40), (41), (42), (43), (44), (45), or (46), having an inner layer and/or an outer layer that additionally comprises a plasticizer.

### BRIEF DESCRIPTION OF THE DRAWINGS

FIG. 1 is a schematic representation of the human large intestine, including the regions: caecum, right colon, transverse colon and descending colon, as well as the sigmoid or rectum (reproduced from Cummings and MacFarlane (1991), J. Appl. Bact. 70(6):443-59).

FIGS. 2A-2B are a schematic representation of certain embodiments of the present invention, including a description of the visualized constructs (FIG. 2A) and a key showing the representation of the symbols (FIG. 2B).

FIG. 3 is a schematic representation of the mechanism of action of a therapeutic agent core which is coated with a microbial-responsive layer.

FIG. 4 is a schematic representation of the mechanism of action of a therapeutic agent core which is coated with a microbial-responsive inner layer and a pH-responsive outer layer.

FIG. 5 is a schematic representation of the mechanism of action of a therapeutic agent core which is coated with a swelling agent inner layer and a microbial-responsive outer layer.

FIG. 6 is a graph showing cumulative bacterial metabolite bound over time by a targeted release (TR) composition of activated carbon particles compared to simulated immediate release (IR) administration of activated carbon particles.

FIGS. 7A-7B are a table and diagram that show demographics of clinical trial participants and trial schedule. FIG. 7A, Trial demographics and metadata summary of participants. FIG. 7B, Phase I clinical trial schedule. Participants were screened during a 4-week run in period, followed by dose escalation in weeks 0-2, 2-4, and 4-8, with a follow-up 4-weeks after trial. Abbreviations: MRI, magnetic resonance imaging; BMI, body mass index; ADOS, Autism Diagnostic Observation Schedule; CGI-S, Clinical Global Impression Severity; PARS, Pediatric Anxiety Rating Scale; 6-GSI, Gastrointestinal Severity Index; BSS, Bristol Stool Scale; NRS, Numerical Rating Scale; GSRS, Gastrointestinal Symptom Rating Scale; ABC-I, Aberrant Behavior Checklist-Irritability; ABC-SW, Aberrant Behavior Checklist-Social Withdrawal; SRS-2, Social Responsiveness Scale; RBS-R, Repetitive Behavior Scale Revised; VABS, Vineland Adaptive Behavior Scales; BL, baseline; EOT, end of treatment, FV, final visit.

FIGS. 8A-8G are a series of graphs that show oral activated carbon particle sequestrant treatment reduces levels of several bacterial metabolites. FIG. 8A-G, Metabolite levels in urine from baseline (BL), end of treatment (EOT), and final visit (FV) timepoints from all participants, normalized to creatinine (g metabolite/mmol creatinine) on log 2 scale. Chemical structures shown above associated data panels. Urine from one subject could not be obtained due to incontinence. Abbreviations: BL, baseline; EOT, end of treatment; FV, final visit; 4EPS, 4-ethylphenyl sulfate; pCG, p-cresol glucuronide; pCS; p-cresol sulfate; 3IS, 3-indoxyl sulfate; HPAA, 3-hydroxyphenylacetate; HPHPA, 3-(3-hydroxyphenyl)-3-hydroxypropionate. Data analysis was conducted on the completers group (n=26, with two missing data points in the FV timepoint because two subjects missed assessment due to parental illness). Analysis is exploratory and post hoc in nature, shown as mean and 95% confidence interval analyzed with a Linear Mixed Effects Model with Geisser-Greenhouse correction, multiple comparisons, and false discovery rate correction across all metabolites (nominal p values: * p adj.≤0.05; ** p adj.≤0.01; *** p adj.≤0.001; **** p adj.≤0.0001).

FIG. 9A-D. Activated carbon particle sequestrant administration improves anxiety and irritability, especially in individuals with high baseline scores. FIG. 9A, Anxiety (measured by PARS) scores of all eligible study participants at baseline (BL), the end of treatment (EOT), and final visit (FV) time points, with mean test scores represented as bars. Dotted line indicates threshold for anxiety (n=24). FIG. 9B, PARS anxiety scores of the subset of individuals scoring ≥10 at BL, showing individual change to EOT and FV (n=15). FIG. 9C, Irritability (measured by ABC-I) scores of all eligible study participants at BL, EOT, and FV time points. Dotted line indicates threshold for the top quartile of irritability severity among the ASD population (BL, EOT n=26; FV n=24 due to a missing final visit value for two individuals). FIG. 9D, Irritability (ABC-I) scores of the subset of individuals scoring ≥15 at BL, showing individual change to EOT and FV (n=11). Abbreviations: PARS, Pediatric Anxiety Rating Scale; ABC, Aberrant Behavior Checklist; BL, baseline; EOT, end of treatment, FV, final visit. Data analysis was conducted on the completers group (n=26, with two missing data points in the FV timepoint in ABC-I and at all time points in PARS because two subjects missed assessments due to parental illness). Analysis is exploratory and post hoc in nature, shown as mean and 95% confidence interval. Analyses were performed by repeated measures one-way ANOVA (FIGS. 9A, B, and D) or the Linear Mixed Effects model (FIG. 9C) with Geisser-Greenhouse correction, multiple comparisons, and false discovery rate correction within each test (nominal p values:* p adj.≤0.05; ** p adj.≤0.01).

FIG. 10A-J. Activated carbon particle sequestrant administration lowers 4EPS levels in gnotobiotic mice and ameliorates anxiety-like behavior. FIG. 10A, Bacterial strain pairs were engineered to produce 4-ethylphenol (4EP+) or not (4EP−). See Methods for details. 4EP is converted to 4EPS by the mouse. FIG. 10B, Timeline schematic for colonization of germ-free mice, sequestrant administration, and metabolite and behavioral analysis. FIG. 10C, Separate groups of mice were each colonized with either the 4EP+ or 4EP− strain pair. 4EPS levels quantified in urine of mice two weeks after dietary administration of sequestrant or control diets. Limit of detection is 10 ng/ml (n number left to right: n=18, 20, 12, 18). FIG. 10D, Weight gain of mice on diet containing 5% sequestrant compared to controls (n=10 each). FIG. 10E, Colonization of mice with 4EP producing strain pairs of Lactobacillus plantarum and Bacteroides ovatus. Both bacterial strains equally colonize 4EP+ and 4EP− groups of mice, independent of diet (n number left to right: n=4, 4, 3, 4). FIG. 10F-J, Behavioral test results from mice administered sequestrant or control diet. FIG. 10F, Visual representations of the behavioral assays open field, elevated plus maze, marble burying and grooming. FIG. 10G, Open field (OF) test results presented as, from left to right, a ratio of time spent in the center of the arena over time spent in thigmotaxis along the perimeter during the 10-minute testing period, total distance mice traveled, time in center, and time in thigmotaxis along the perimeter of the arena (n number left to right: n=31, 33, 26, 26). FIG. 10H, Elevated plus maze (EPM) results presented, from left to right, as a ratio of time spent in the open arms of the maze over time spent in the closed arms during the 5-minute testing period, time mice spent in the open arms, time spent in the closed arms, and total time at the terminus (outermost third of open arms)(n number left to right: n=27, 29, 23, 25). FIG. 10I, Number of marbles buried during the 10-minute testing period (n number left to right: n=31, 33, 25, 26). FIG. 10J, Amount of time spent grooming during the 10-minute testing period (n number left to right: n=26, 32, 22, 25). Abbreviations: 4EP, 4-ethylphenol; 4EPS, 4-ethylphenyl sulfate. Data represent mean±SEM analyzed by ordinary two-way ANOVA test with FDR correction, with individual variances computed for each comparison (* p adj.≤0.05; ** p adj.≤0.01; *** p adj.≤0.001; **** p adj.≤0.0001).

FIG. 11A-C. Consort flow diagram, CGI-S, and CGI-I data in clinical samples. FIG. 11A, A total of 41 individuals were screened for eligibility across 3 sites in New Zealand and Australia between April 2019 and January 2020. 30 participants were enrolled following meeting predefined criteria for study (see methods and Table 2). 27 participants completed treatment and 25 completed the follow up visit. FIG. 11B, Clinical global impression improvement scores (CGI-I) for all participants at visit 4, EOT and FV time points (n=26 with two missing participants for FV due to parental illness). All participants' scores were normalized to a 4 at BL, thus this data is relative to 4; lower number=improvement, higher number=worsening. FIG. 11C, Clinical global impression severity (CGI-S) score distribution for completers group at baseline (BL), visit 2, visit 4, end of treatment (EOT), and final visit (FV) time points (n=26 with two missing participants for FV due to parental illness). Abbreviations: CONSORT, Consolidated Standards of Reporting Trials; CGI-S, clinical global impression severity; CGI-I, clinical global improvement; rACC2, rostral anterior cingulate cortex; BL, baseline; V2, visit 2; V4, visit 4; EOT, end of treatment; FV, final visit.

FIG. 12A-D. Quantitative values of metabolites in plasma and correlations between urine and plasma metabolite levels. FIG. 12A, Metabolite levels in urine that were not altered from baseline (BL), end of treatment (EOT), and final visit (FV) timepoints from the completers group, normalized to creatinine (g metabolite/mmol creatinine) on log 2 scale. Chemical structures shown above associated data panels. Urine from one subject could not be obtained due to incontinence, and two participants did not complete the FV timepoint due to parental illness (BL, EOT n=25; FV n=23). HPPA measured below the limit of quantification in ≥50% of individuals; no imputation was performed, missing values not shown. FIG. 12B, Quantitative values of metabolites in plasma samples at baseline (BL) and end of trial (EOT) timepoints with ng/ml levels (log 2 scale) along the y-axis (n=26). FIG. 12C, Pearson correlation between plasma (ng/ml) and urine (ug/mmol creatinine) levels for each measured metabolite (n=25). FIG. 12D, Quantitative values of control metabolite, N-acetyl serine (N-AS) in urine and plasma samples. Abbreviations: BL, baseline; EOT, end of treatment; HPPA, 3-(4-hydroxyphenyl)propionate; HHA, 3-hydroxyhippurate; CMPF, 3-carboxy-4-methyl-5-propyl-2-furanpropanoate; IPA, imidazole propionate; 4EPS, 4-ethylphenyl sulfate; pCG, p-cresol glucuronide; pCS; p-cresol sulfate; 3IS, 3-indoxyl sulfate; HPHPA, 3-(3-hydroxyphenyl)-3-hydroxypropionate; HPPA, 3-(4-hydroxyphenyl)propionate; HHA, 3-hydroxyhippurate; HPAA, 3-hydroxyphenylacetate; N-AS, N-acetyl serine. Data analysis was conducted on the completers group. Data analysis is exploratory and post hoc in nature, shown as mean and 95% confidence intervals in panels a and c, analyzed by a two-tailed paired t-test or ANOVA with multiple comparisons and false discovery rate correction as appropriate (* p≤0.05; ** p≤0.01; *** p≤0.001) and a Pearson's correlation in FIG. 12B.

FIG. 13A-E. Altered amygdalar functional connectivity and Vineland Adaptive Behavior Scales (VABS) diagnostic scores. FIG. 13A, Functional connectivity between the amygdala and rACC brain regions, assessed by fMRI scans at BL and EOT. VABS test scores at baseline (BL), the end of treatment (EOT), and final visit (FV) timepoints, where a lower score indicates increased severity. Dotted line indicates threshold (≤86) of scores categorized as moderately low and low (n=8). FIG. 13B-D, Vineland Adaptive Behavior Scales (VABS). FIG. 13B, Composite scores (n=15). FIG. 13C, Communication scores (n=15). FIG. 13D, Daily living scores (n=15). FIG. 13E, Socialization scores (n=15). In each respective left panel, scores of all study participants with valid VABS are shown, with a dotted line marking the threshold of 86 for moderately low and low scoring individuals. Mean increase between BL and EOT timepoints is noted. Abbreviations: rACC, rostral anterior cingulate cortex; BL, baseline; EOT, end of treatment, FV, final visit. Data analysis was conducted on the completers group, but any individual with over 25% estimated answers in any domain was removed. Data analysis is exploratory and post hoc in nature. FIG. 13A is a subset of the participants who agreed to fMRI, analyzed by a paired t-test, shown, and panels b-e are displayed as mean and 95% confidence intervals with analysis performed by Linear Mixed Effects Analysis with multiple comparisons and false discovery rate correction (* p adj.≤0.05, ** p adj.≤0.01, *** p adj.≤0.001).

FIG. 14A-J. Extended behavior scores of the social responsiveness scale (SRS) and Aberrant Behavior Checklist (ABC). FIG. 14A-F, SRS behavior scores (n=24) at baseline (BL), the end of treatment (EOT), and final visit (FV) timepoints for the composite score (FIG. 14A), repetitive behavior (FIG. 14B), social motivation (FIG. 14C), social awareness (FIG. 14D), social communication (FIG. 14E), and social cognition (FIG. 14F) domains. FIG. 14G-J, ABC behavior scores at BL, EOT, and FV for all individuals for the stereotypic behavior score (BL, EOT, n=26; FV, n=24) (FIG. 14G), inappropriate speech (FIG. 14H), hyperactivity/noncompliance (FIG. 14I), and social withdrawal (FIG. 14J) domains. Dotted lines indicate categorical thresholds for the top quartile among the ASD population. Mean increase between BL and EOT timepoints is noted. Abbreviations: BL, baseline; EOT, end of treatment; FV, final visit. Data analysis was conducted on the completers group minus two subjects who missed assessment due to parental illness. Data analysis is exploratory and post hoc in nature, shown as mean and 95% confidence intervals with statistics for performed by repeated measures one-way ANOVA with multiple comparisons and false discovery rate correction. (* p adj.≤0.05, ** p adj.≤0.01, *** p adj.≤0.001).

FIG. 15. Squared Partial Correlation of Change in Score vs. Change in Biomarkers Controlling for Baseline Score ABC-I Assessment.

FIG. 16. Squared Partial Correlation of Change in Score vs. Change in Biomarkers Controlling for Baseline Score PARS Assessment.

FIG. 17. Models Using Baseline Score+Top (N/3)−1 Specimen/Biomarkers as Covariates Change in Score vs. Change in Biomarkers.

FIG. 18. Squared Partial Correlation of Change in Score vs. Baseline Biomarkers Controlling for Baseline Score ABC-I Assessment.

FIG. 19. Squared Partial Correlation of Change in Score vs. Baseline Biomarkers Controlling for Baseline Score PARS Assessment.

FIG. 20. Models Using Baseline Score+Top (N/3)−1 Specimen/Biomarkers as Covariates Change in Score vs. Baseline Biomarkers.

FIG. 21A-C. FIG. 21A shows a representation of Eudragit NM 30D and Pectin, 043021-1 Films. FIG. 21B shows a representation of Eudragit NM 30D and Soy Polysaccharides, 043021-2 Films. FIG. 21C shows a representation of Eudragit NM 30D and Guar Gum, 043021-3 Films.

FIG. 22. Representation of well plates secured inside of the polycarbonate box,

FIG. 23. Representation of Plate I.

FIG. 24. Representation of Plate II.

FIG. 25. p-cresol adsorption by AB2004 coated with 100% Surelease.

FIG. 26. p-cresol adsorption by AB2004 coated with 20% High Amylo N-400+Surelease.

FIG. 27. p-cresol adsorption by AB2004 coated with 10% Pea Protein+Surelease.

FIG. 28. p-cresol adsorption by AB2004 coated with 10% Nutriose+Surelease.

FIG. 29. Amount of p-cresol adsorbed by coated activated carbon particle sequestrant in the presence of SHIME.

FIG. 30. Amount of indole adsorbed by coated activated carbon particle sequestrant in the presence of SHIME.

FIG. 31. Amount of p-cresol adsorbed by coated activated carbon particle sequestrant in the presence of SHIME—untreated.

FIG. 32. Amount of p-cresol adsorbed by coated activated carbon particle sequestrant in the presence of SHIME—treated.

FIG. 33. Amount of indole adsorbed by coated activated carbon particle sequestrant in the presence of SHIME—untreated.

FIG. 34. Amount of indole adsorbed by coated activated carbon particle sequestrant in the presence of SHIME—treated.

FIG. 35. Localization of bile acids in the GI tract and examples of primary and secondary bile acids.

FIG. 36A-D. Results for a series of semi-synthetic glucosamine based cationic polymers. Polymer A (FIG. 36A), Polymer B (FIG. 36B), Polymer C (FIG. 36C), Polymer D (FIG. 36D).

FIG. 37. Physical properties of semi-synthetic glucosamine polymers.

FIG. 38. provides data showing that Polymer B shows similar preference for conjugated secondary bile acid glycodeoxycholic acid (GDCA) vs. primary conjugated bile acid glycocholic acid (GCA).

FIG. 39. provides data showing that Polymer B shows binding affinity to chenodeoxycholic acid (CDCA) as well as its glycol and tauro conjugated forms FIG. 40. provides examples of R1 groups for active capped cyclodextrins for incorporation into glucosamine polymers for targeting adsorption of secondary bile acids and generation of semi-synthetic polymers with glucosamine backbone.

FIG. 41. Preparation of NPs via a dispersion copolymerization and the structures of hydrophobic monomers adopted for capturing indole.

FIG. 42. provides examples of bile acid sequestrants.

FIG. 43. shows duplicate p-cresol binding obtained for 3 lots. Under the test conditions, at 24 h time point, not less than 178 mg and not more than 242 mg of p-cresol is bound per g of activated carbon particles.

### DETAILED DESCRIPTION

### Definitions

All scientific and technical terms used herein, unless otherwise defined below, are intended to have the same meaning as commonly understood by one of ordinary skill in the art to which this invention belongs. In the case of any conflict, the present specification, including definitions, will control. References to techniques employed herein are intended to refer to the techniques as commonly understood in the art, including variations on those techniques or substitutions of equivalent or later-developed techniques which would be apparent to one of skill in the art. In order to more clearly and concisely describe the subject matter which is the invention, the following definitions are provided for certain terms which are used in the specification and appended claims.

As used herein, a “therapeutic agent” refers to a compound or complex having a therapeutic effect on a living organism, or system or cell thereof, such as a drug, a drug substance, a drug product or an active pharmaceutical ingredient (API). The therapeutic agent can be water soluble or insoluble. The therapeutic agent may or may not be absorbed into the living organism, or system or cell thereof. The therapeutic agent can act directly or indirectly on the living organism, or system or cell thereof.

As used herein, a “therapeutic agent core” of the pharmaceutical compositions of the invention means a volume comprising or consisting of the therapeutic agent, which is contained within at least one (e.g., at least one, at least two, at least 3, or at least 4 or more) layers which substantially surround and enclose the core. The core can be spherical, or approximately or substantially spherical, or can be irregularly shaped (e.g., globular). The therapeutic agent core can be crystalline or amorphous. The therapeutic agent core can be solid or porous, or can comprise a multiplicity of particles which are solid or porous. The therapeutic agent core can comprise multiple therapeutic agents (e.g., sequestrants, antibiotics, anti-inflammatory agents) as well as non-therapeutic agents (e.g., binders, swelling agents).

As used herein, a “therapeutic agent core surface” means the outermost surface of the volume defining the core which is in contact with the innermost layer until the innermost layer is dissolved, degraded or bursts.

As used herein, the terms “layer” and “coating” are used interchangeably and refer to a substantially continuous three-dimensional coating comprising a polymeric material or swelling agent, or a mixture of at least one polymeric material and one or more or non-polymeric materials, that substantially covers, encloses or surrounds a therapeutic agent core of the invention and, optionally, additional layers (e.g., of polymeric materials and/or swelling agents). The layers of the invention can be solid or porous, water-permeable or water-impermeable, protease-degradable or protease resistant, pH-responsive or pH-nonresponsive. The layers of the invention can be referred to as inner or outer, or first inner, second inner, third inner, etc., or first outer or second outer, etc. In each case, an inner layer is understood to be closer to the therapeutic core than an outer layer, a first inner is understood to be closer to the therapeutic core than a second inner, or third inner, etc., and a first outer is understood to be closer to the therapeutic core than a second outer, or third outer, etc.

As used herein, a “delayed-burst release” is defined herein as a release of greater than about 20% of the therapeutic agent of a pharmaceutical composition for oral administration, after a defined time interval. In some embodiments, a colon-activated delayed-burst release has an approximate time interval which starts after the dosage form enters the colon. In some embodiments, the colon-activated delayed-burst release dosage form is a dosage form that releases less than about 20% of its contents in simulated upper-GI conditions over a 24 h time frame. In some embodiments, upon entering the colon, a delayed-burst release formulation can release at least 20% of a coated therapeutic agent in or around a targeted time frame, for example about 1 h after entering the colon; about 3 h after entering the colon; about 5 h after entering the colon; about 7 h after entering the colon; about 9 h after entering the colon, and the like. In some embodiments, the different time frames of release provide the utility of targeting differential regions of the colon.

As used herein, the term “swelling agent” refers to a three-dimensional network of hydrophilic polymer chains that are chemically or physically cross-linked. Swelling agents can absorb water (for example, absorbing water from an aqueous solution) or organic solutions, leading to an increase in volume. After absorbing a solvent, a swelling agent can increase in volume by at least 10% (e.g., at least 10%, at least 20%, or 30% or more) relative to its original volume. As used herein, the terms “pH-responsive polymer” or “pH-responsive layer” or “pH-responsive coating” refer to a polymeric layer or coating that changes its solubility in response to a pH level, or a change in pH level, with at least a 10× change in solubility from a pH in which it is insoluble to a pH in which it is soluble. In the context of the present disclosure, the pH level at which solubility changes can be pH 6.5-7.5 or any value therein, such that the pH-responsive layer is substantially insoluble in the stomach or small intestine, but becomes substantially more soluble in the caecum or colon.

As used herein, the terms “microbial-responsive polymer” or “microbial-responsive layer” or “microbial-responsive coating” refer to a polymeric layer or coating that can be degraded (e.g., proteolyzed, hydrolyzed) by one or more enzymes (e.g., proteases, hydrolases, glycosidases) released by the microbial biome. Thus, a microbial-responsive layer can be degraded in the colon where the microbiome is most plentiful.

The term “binding agent” (or “binding agents”) is an art-recognized term, and is typically a material that holds other materials together through physical, physicochemical and/or hydrogen-bonding interactions, and the like.

As used herein, the term “adsorbent” refers to a compound or material that is capable of adsorbing one or more toxins or deleterious bacterial metabolites and, therefore, acts as a therapeutic agent.

As used herein, the term “activated carbon adsorbent” refers to an activated carbon particle preparation that is capable of adsorbing one or more toxins or deleterious bacterial metabolites and, therefore, acts as a therapeutic agent. An example of an activated carbon adsorbent is provided in WO2018183986, which description is incorporated herein by reference in it's entirety. In some embodiments, the activated carbon adsorbent comprises substantially spherical particles having a minimum average specific surface area determined by the Brunauer-Emmett-Teller (BET) method of at least 500 m2/g and a maximum average specific surface area determined by the Brunauer-Emmett-Teller (BET) method less than 4000 m2/g. In some embodiments, the activated carbon adsorbent comprises substantially spherical particles having a minimum average particle diameter of at least 0.005 mm and a maximum average particle diameter of less than 1.5 mm.

In the present disclosure, the terms “activated carbon adsorbent” or “activated carbon particles” or “activated carbon sequestrant” are used interchangeably and refer to a substantially spherical microparticulate therapeutic agent primarily composed of activated carbon. Activated carbon particle preparations are commercially available from various suppliers, including the products Kremezin® or AST-120 (Kureha Corp., Tokyo, JP), Merckmezin® or Mylan (Merck Hoei Ltd., Osaka, JP), and Kyucal® (Nichi-Iko Corp., Toyama, JP).

In the present disclosure, the phrase “for exposing a surface of the therapeutic agent for the first time in the colon” means that the surface is covered with a plurality of layers, in some embodiments comprising an enteric polymer-containing layer, and/or contained within an enteric capsule, before oral administration, and after oral administration, the surface of the therapeutic agent is substantially covered before the descending colon is reached, and then subsequently uncovered. It is known that the pH in the stomach fluctuates between pH values of about 1 to 6.5 during the day, and the pH fluctuates between about 5 to 6.5 in the upper part of the small intestine including the duodenum.

Accordingly, the phrase “the surface is substantially covered before the descending colon, which is the target site, is reached” means that the adsorption performance for intestinal metabolites, and precursors thereof, or concomitantly-administered or used other drugs or therapeutic agents, in an acidic to weakly acidic environment is suppressed to less than 40% and, in some embodiments, less than 20%, compared with a therapeutic agent that is not provided with said means. The phrase “uncovered when the target site is reached” means that the adsorption performance of the therapeutic is recovered (reactivated) in a neutral environment.

In some embodiments, examples of the “means for exposing a surface of the therapeutic agent for the first time in the colon” or the “means for exposing for the first time in the colon” used in the present disclosure include enteric release preparations, delayed release preparations, sustained release preparations, or large-intestine release preparations using an intestinal bacteria-soluble polymer such as pectin, starch, or the like, in which an enteric base that dissolves site-specifically in the digestive tract from a lower part of the small intestine to the descending colon is used, and dosage forms such as tablet preparations, capsule preparations, and granule preparations are provided. Among other embodiments, in particular, enteric release preparations, delayed release preparations, and large-intestine release preparations using an intestinal bacteria-soluble polymer such as chitosan are provided enabling drug delivery specifically to the large intestine or descending colon according to the properties and the amount of the polymer used.

In some embodiments, the “means for exposing for the first time in the colon” used in the present disclosure includes 1) coating a therapeutic agent with a colon-targeting polymer, and 2) encapsulating a therapeutic agent that can be coated with an enteric polymer, within an enteric capsule.

In other embodiments, the “pharmaceutical composition, wherein the means for exposing a therapeutic agent core surface for the first time in the colon is provided” of the present disclosure includes:
a) a granule preparation, a capsule preparation, or a tablet preparation comprising a therapeutic agent coated with a colon-targeting polymer;
b) a capsule preparation wherein a therapeutic agent is encapsulated within a colon-targeting capsule; and
c) a tablet preparation wherein a compression-molded product of a therapeutic agent is coated with a colon-targeting polymer.

In some embodiments, the “granule preparation” of the present disclosure includes granule preparations, fine granule preparations, or powder preparations set forth in the United States Pharmacopoeia (USP).

As used herein, the term “polysaccharide” refers to a polymer of carbohydrate monomers (e.g., starch, cellulose, glycogen) that can be linear or branched.

As used herein, the term “protein” refers to a polymeric macromolecule comprising one or more polymer chains of amino acid residues. Proteins can also comprise intrachain or interchain crosslinking, as well as covalent modifications with other moieties (e.g., glycans).

As used herein, the term “vegetable protein” refers to a protein preparation isolated from one or more vegetables which have been dried and ground into a fine flour. A substantial amount of the starch and fiber content can be removed by washing with water or other solvents, optionally using enzymes to improve isolation of the vegetable proteins. Similarly, as used herein, the terms “pea protein” or “maize protein” refer to preparations of vegetable protein isolated from peas (e.g., yellow pea or Pisum Sativum) or maize (e.g., Zea mays) which have been dried and ground into a fine flour. A substantial amount of the starch and fiber content can be removed by washing with water or other solvents, optionally using enzymes to improve isolation of the vegetable proteins. For example, in commercially available preparations, pea protein can comprise about 85% of the dry weight of the preparation (e.g., NUTRALYS™ S85F, Roquette Freres, Lestrem, France). Similar preparations can be produced from other vegetables, including peas, beans, lentils, legumes, and maize and constitute equivalents of pea protein.

As used herein, the term “polymer” refers to a substance that has a molecular structure consisting primarily or entirely of a large number of subunits, called “monomers”, that are covalently bound together to form a linear or branched chain. A polymer can comprise many identical subunits (e.g., polylactic acid polymers), or a mixture of many different types of subunits (e.g., polysaccharides, proteins), often in a repeating fashion.

The term “water insoluble” means that a substance is incapable of dissolving in water (or being dissolved by water) or is only sparingly soluble in water (e.g., having a solubility in water of less than 1 mg/mL).

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the recitation of a numerical range for a variable is intended to convey that the invention can be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### Principles of the Invention

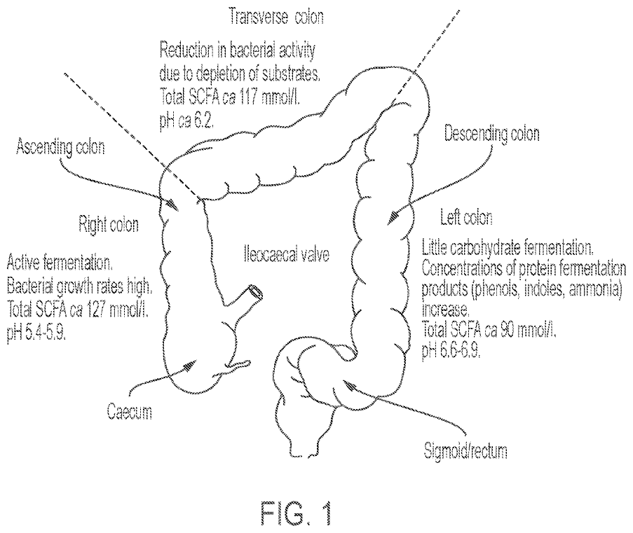

As shown in FIG. 1, the human colon is roughly divided into three compartments. Currently, there is no targeting mechanism available or published for differential delivery of therapeutic agents to regions within the colon.

The present disclosure provides materials and methods based, at least in part, on the following findings:
(a) A microbial-responsive layer (e.g., insoluble protein) enclosing a therapeutic agent core is digested by the proteolytic activity of bacteria naturally present in the colonic microbiome, thereby releasing the therapeutic agent in the colon; such microbial-responsive) (e.g., protease-degradable, hydrolase-degradable, glycosidase-degradable) layers can be composed of poorly-digestible protein (e.g., zein proteins) or digestible protein (e.g., partially hydrolyzed zein, hydrolyzed zein, pea protein, soy proteins, egg proteins, gelatin, etc.) and/or poorly digestible polysaccharides or crosslinked proteins or polypeptides (e.g., polyaminoacids, including poly-L-glutamic acid, poly-L-lysine, poly-L-aspartic acid, polyglucosamines, etc.).
(b) In order to avoid release of the therapeutic agent by proteases in the upper gastrointestinal tract (GIT), an outer layer is provided which encloses a microbial-responsive inner layer and the therapeutic agent core. The outer layer can comprise one or more pH-responsive polymers which are stable in the upper GIT but can dissolve at the higher pH levels in the colon (e.g., pH 6.5-7) and/or can comprise poorly digestible or poorly hydrolyzable polysaccharides which are not substantially degraded during transit to the colon. Thus, in some embodiments, the pH-responsive polymeric outer layer protects the microbial-responsive (e.g., protease-degradable) inner layer in the digestive part of small intestine. Upon reaching the ileum, a pH-responsive polymer coating dissolves, exposing the microbial-responsive inner layer.
(c) Alternatively, a poorly host-digestible or hydrolyzable polysaccharide outer layer provides a hydrated polysaccharide layer to protect the microbial-responsive inner layer from digestion or hydrolysis. Upon reaching the caecum, the polysaccharide is fermented or digested by bacterial enzymes, leaving the microbial-responsive inner layer susceptible to degradation (e.g., by microbial enzyme activity).
(d) A layer of swelling agent can be used to achieve delayed-release of a therapeutic agent in the ascending colon. Because it can be desirable to target some therapeutic agents to the distal end of the ascending colon, the present disclosure further provides compositions and methods for delayed-release of a therapeutic agent after entering the colon. This is accomplished by the presence of a swelling agent layer. When an outer layer enclosing a swelling agent layer begins degrading due to pH or enzymatic activity, the partially-degraded coating allows permeation of water into the swelling agent layer. The permeated water causes swelling of the swelling agent layer, causing the outer layer to burst, thereby releasing therapeutic agent. Control over the amount of swelling agent, rate of water permeation and thickness of the outer layer controls the time period before the outer layer bursts. As gastrointestinal transit moves the pharmaceutical composition further along the large intestine, this effect provides a time-controlled means for burst-release, allowing targeting of therapeutic agent to the colonic location.

The materials, compositions, and methods provided herein are useful for localized delivery of a therapeutic agent into the GI tract of a subject.

The current disclosure provides materials and methods for a more specifically-targeted release profile occurring well past the ileum/caecum entry points of the colon. Using materials and methods provided herein, coupled with adsorption experimental data described herein, this disclosure provides a specific site-targeted release of a therapeutic agent core to the colon. Further, the present disclosure provides materials and methods for utilizing partially-degraded, partially-digested or partially-hydrolyzed film-forming materials which impart unique colon-targeted delivery properties, as well as swelling agent layers to control release from a slowly dissolving coating or layer.

### Controlled-Release Compositions for Delivery to the GI Tract

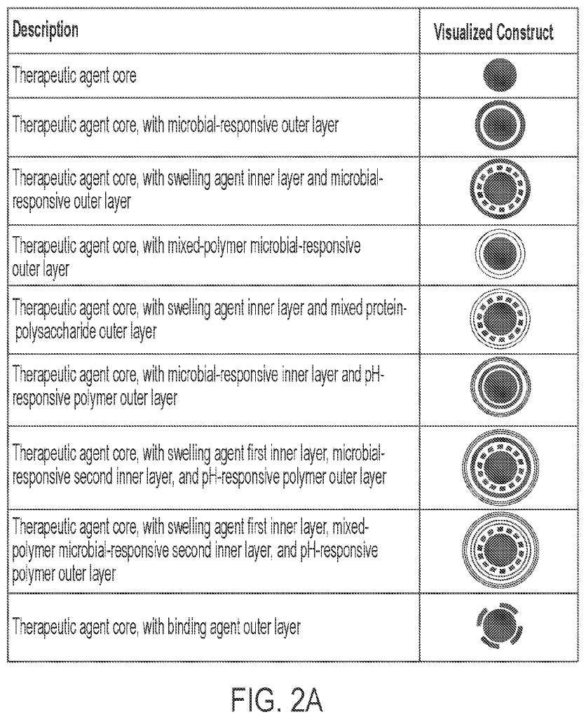

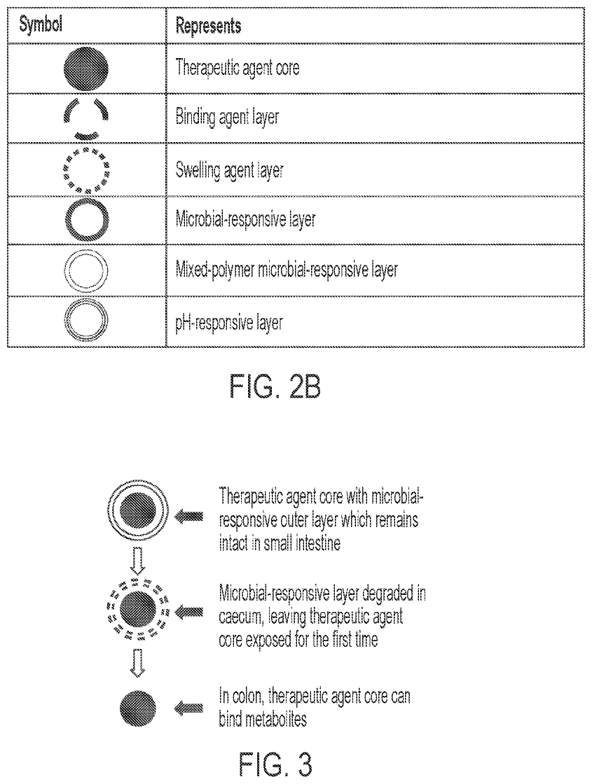

As shown in FIG. 2, in some embodiments, the pharmaceutical compositions of the invention comprise a therapeutic agent core and:
(1) a microbial-responsive outer layer;
(2) a swelling agent inner layer and a microbial-responsive outer layer;
(3) a mixed protein-polysaccharide outer layer;
(4) a swelling agent inner layer and a mixed-polymer (e.g., protein-polysaccharide or water-insoluble polymer-polysaccharide) microbial-responsive outer layer;
(5) a microbial-responsive inner layer and a pH-responsive outer layer;
(6) a swelling agent first inner layer, a microbial-responsive second inner layer and a pH-responsive polymer outer layer;
(7) a swelling agent first inner layer, a mixed-polymer (e.g., protein-polysaccharide or water-insoluble polymer-polysaccharide)microbial responsive inner layer and a pH-responsive polymer outer layer; and
(8) a binding agent outer layer, which may be added to the therapeutic agent core in any of the foregoing embodiments.

In each of the foregoing embodiments, additional layers can be interposed provided that (a) there is at least one “outer” layer which protects at least one “inner” layer from degradation in the upper GI tract (i.e., stomach, small intestine), and (b) all “outer” and “inner” layers have been degraded at a location in the colon such that the therapeutic agent is delivered to the tissues of the colon and/or colonic microbiome. As discussed herein, delayed-burst of one or more of the inner layers can be used to deliver the therapeutic agent further along in the colon, including to the distal ascending colon.

FIG. 3 depicts the use of one pharmaceutical composition of the invention. A pharmaceutical composition is provided in which a therapeutic agent core is enclosed in an outer layer comprising a microbial-responsive layer (e.g., a mixture of hydrolyzed zein protein and a polysaccharide). This layer is not degraded in the upper GI because the microbial-responsive layer (e.g., zein-based coating) is not easily digested/hydrolyzed. In the caecum or further along in the colon, however, the microbial-responsive layer is degraded (e.g., polysaccharide components of the outer layer are hydrolyzed and the zein is subject to proteolytic degradation), releasing the therapeutic agent.

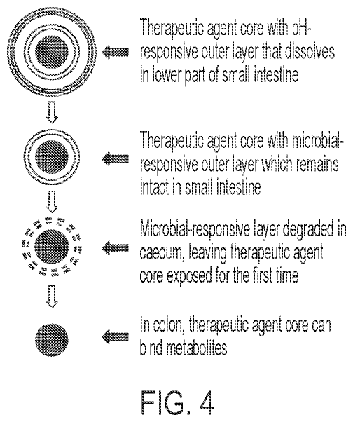

FIG. 4 depicts the use of another pharmaceutical composition of the invention. A pharmaceutical composition is provided in which a therapeutic agent core is surrounded by an outer pH-responsive polymer outer layer that does not dissolve until reaching the lower part of the small intestine. Within the outer layer is a microbial-responsive inner layer (e.g., a mixture of zein protein and polysaccharide). This layer remains substantially intact until the composition transits to the caecum, where the microbial-responsive component is degraded (e.g., the polysaccharide is hydrolyzed and the zein protein is proteolyzed), releasing the therapeutic agent.

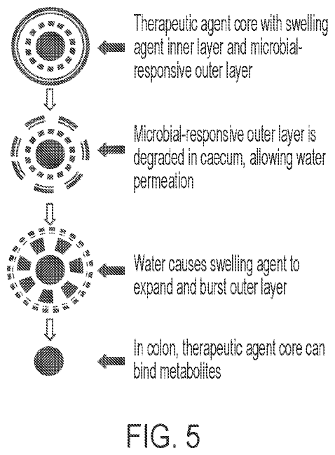

FIG. 5 depicts the use of another pharmaceutical composition of the invention. A pharmaceutical composition is provided in which a therapeutic agent core is surrounded by an inner layer of a swelling agent and an outer layer comprising a microbial-responsive layer. The outer layer remains substantially intact throughout transit in the upper GI, but the degraded in the caecum. As the outer layer is degraded, water can permeate that layer and cause the swelling agent to expand, leading to bursting of the outer layer and release of the therapeutic agent.

In certain embodiments, the pharmaceutical preparation of the present disclosure is “a pharmaceutical composition, wherein an amount of an adsorbate adsorbed in an adsorption test of the pharmaceutical composition is less than 40% and some embodiments less than 20% in an environment having a pH of 5 or less, and 50% or more and in some embodiments, 60% or more in an environment having a pH of 7 or more based on an amount of an adsorbate adsorbed by a therapeutic not provided with the means”. Here, p-cresol, p-cresol sulfate (p-cresyl sulfate), 4-ethyl phenol, and the like are used as adsorbates. The adsorption test can be performed by the following adsorption test method using p-cresol, or the method of Example 4 or 13 described below.

In some embodiments, a first or second inner layer comprises a colon-targeting polymer; in some embodiments, a first outer layer comprises a colon-targeting polymer; in certain other embodiments, an inner or an outer layer of colon-targeting polymer is provided along with a separate layer of an enteric polymer or enteric capsule, that dissolves at a pH of about 5 to 8, or at about a pH of 5 to 7.

The “colon-targeting” or “colon-targeted” polymers used in the present disclosure are not particularly limited, and examples include one or more acrylic-based, cellulose-based, vinyl-based polymers, and the like, alone or in combination, among other examples, which are selected to preferably target delivery of a therapeutic agent to the descending colon.

### Polymeric Layers

In some embodiments, a first or second inner layer comprises a colon-targeting polymer; in some embodiments, a first outer layer comprises a colon-targeting polymer; in certain other embodiments, an inner or an outer layer of colon-targeting polymer is provided along with a separate layer of an enteric polymer or enteric capsule, that dissolves at a pH of about 5 to 8, or at about a pH of 5 to 7.

The “colon-targeting” or “colon-targeted” polymers used in the present disclosure are not particularly limited, and examples include one or more acrylic-based, cellulose-based, vinyl-based polymers, and the like, alone or in combination, among other examples, which are selected to preferably target delivery of a therapeutic agent to the descending colon.

Examples of the acrylic-based polymer include (1) a copolymer of methacrylic acid and ethyl acrylate, (2) a copolymer of methacrylic acid and methyl methacrylate, or (3) a copolymer of methacrylic acid, methyl acrylate, and methyl methacrylate. (1) The copolymer of methacrylic acid and ethyl acrylate is listed in the United States Pharmacopeia (USP) as methacrylic acid copolymer LD, and is commercially available from Evonik Industries AG under the product name Eudragit L30D-55. (2) The copolymer of methacrylic acid and methyl methacrylate is described in the “Japanese Pharmaceutical Excipients” as a representative enteric polymer. Those having a methacrylic acid content of 27.6% to 30.7% (hereinafter also referred to as methacrylic acid copolymer S) and 46.0 to 50.6% (hereinafter also referred to as methacrylic acid copolymer L) can be used as the enteric polymer of the present disclosure, for some embodiments. Methacrylic acid copolymer L and methacrylic acid copolymer S are commercially available from Evonik Industries AG under product names: Eudragit L100 (methacrylic acid copolymer L) and Eudragit S100 (methacrylic acid copolymer S). (3) The copolymer of methacrylic acid, methyl acrylate, and methyl methacrylate is commercially available from Evonik Industries AG under product name: Eudragit FS30D.

Examples of the cellulose-based polymer include cellulose acetate phthalate, cellulose acetate trimellitate, hydroxypropyl methylcellulose phthalate (hereinafter also referred to as hypromellose phthalate), hydroxypropyl methylcellulose acetate succinate (hereinafter also referred to as hypromellose acetate succinate or hypromellose acetic acid ester succinic acid ester), carboxymethyl ethylcellulose, cellulose acetate succinate, methylcellulose phthalate, hydroxymethyl cellulose phthalate, hydroxypropyl methyl acetate maleate, and hydroxypropyl methyl trimellitate, among others; cellulose acetate phthalate, hypromellose phthalate, and hypromellose acetate succinate, in certain embodiments. Hypromellose acetate succinate is commercially available from Shin-Etsu Chemical Co., Ltd., under product name Shin-Etsu AQOAT® in several grades having different pH solubilities such as HPMC-AS-HG or, as AquaSolve™ by Ashland.

Examples of the vinyl-based polymers include polyvinyl acetate phthalate and polyvinyl butyrate phthalate, and the like, among others.

Examples of enteric polymers include methacrylic acid copolymer L, methacrylic acid copolymer S, methacrylic acid copolymer LD, and hydroxypropyl methylcellulose acetate succinate, and the like, among others.

Examples of proteins include gelatins, albumins, soy proteins, pea proteins, maize proteins (such as zein), collagens, and the like, among others. In certain embodiments, these proteins can be partially-digested or partially-hydrolyzed.

Examples of polysaccharides include chitosan, resistant starches, alginate, alginates, amylose, amyloses, galactomannans, arabinoxyan, inulin, inulins, pectins, guar gums, xanthan gums, chondroitin sulfate, dextrans, cyclodextrins, Nutriose® brand soluble fiber, locust bean gums, soy polysaccharides, pea polysaccharides, fructo-oligosaccharides and the like, among others.

Examples of water insoluble polymers include ethylcellulose, poly methylmethacrylate, polyvinyl chloride, and the like, among others.

Examples of swelling agents include cellulose derivatives including HPMC, MC; hydrocolloids including alginate, chitosan, pectin, poly (ethylene oxide), carbopol, poly (vinyl alcohol), crosslinked sodium carboxymethylcellulose, crosslinked poly (vinyl pyrrolidone), sodium starch glycolate, acrylamide, acrylic acid, 2-hydroxyethyl methacrylate, and the like, among others.

In some embodiments, one or more “anti-tacking agent” is used in a coating system to prevent or eliminate self-adhesive properties (for example stickiness) of one or more components of a dosage form during the manufacturing process. In some embodiments, anti-tacking agents include talc, talcum, magnesium stearate, glyceryl monostearate, and the like.

Two or more enteric polymers can be used in combination in a variety of proportions. In some embodiments, enteric polymers are combined such that the polymers dissolve at a pH of 5 to 8; in other embodiments, a pH of 5 to 7. In some embodiments, a combination of methacrylic acid copolymer L and methacrylic acid copolymer S can be used, with the methacrylic acid copolymer S in an amount of 0.3 to 5 times and in an amount of 1 to 3 times methacrylic acid copolymer L in terms of weight ratio.

In some embodiments, two or more colon-targeting polymers can be used in combinations, in a variety of proportions.

In some embodiments, one or more colon-targeting polymer or polymers are combined with a lubricant, a plasticizer, a pigment, an anti-tacking agent and the like as necessary, and the combination is used for one or more layers. Examples of the lubricant to be added include, among others, talc, and examples of the plasticizer include, among others, triethyl citrate, and/or dibutyl sebacate.

### Therapeutic Agent Cores

### 1. Adsorbents as Therapeutic Agent Cores to Adsorb One or More Toxins or Metabolites in the GI Tract

Activated carbon preparations have been investigated to counteract the effects of uremic toxins in the treatment of chronic kidney disease (CKD) (e.g., Schulman et al. (2016), BMC Nephrology 17:141). For example, an activated carbon particle preparation has been developed and utilized for delaying dialysis in subjects suffering from chronic kidney disease (AST-120, Kureha Corp., Tokyo, JP).

It has been reported that increased levels of bacterial metabolites, such as p-cresyl sulfate (PCS), 4-ethyl-phenyl sulfate (4-EPS), and 3-indoxyl sulfate (IS), have been detected in subjects with autism spectrum disorder (ASD). Recently, treatments have been developed using orally administered activated carbon particle adsorbents to lower the levels of bacterial metabolites for the treatment of ASD populations.

Although activated carbon adsorbents have been prescribed to improve uremic symptoms and delay dialysis initiation, their use places great constraints on a subject's medication adherence. First, depending on the types of other drugs (concomitant drugs) taken by the subject, activated carbon adsorbent can adsorb other drugs at a stage before other drugs are absorbed into the body. Therefore, there are limitations on the dosing timing of spherical carbon adsorbent, resulting in reduced subject compliance. A reminder is provided to avoid taking the activated carbon adsorbent simultaneously with other drugs, and to take the activated carbon adsorbent 30 minutes, 1 hour or more after taking other drugs. Second, the dosage of the existing activated carbon adsorbent is large (2 g three times/day, or 6 g/day), which causes difficulty in subject compliance. In the case of a capsule preparation, up to 10 capsules have to be taken at a time, for certain indications.

Given that most adsorbents are non-specific in their binding characteristics (specificity, selectivity, etc.), their immediate release forms have the opportunity to bind many molecules in the GIT, including co-administered medications, digestive enzymes, bile acids, food digestion products—amino acids, peptides, fatty acids, phosphates, oxalates, etc. Indeed, co-administration of medication is contra-indicated for many oral adsorbents. The present disclosure provides materials and methods that recognize that site of absorption of medications and nutrients is separate and upstream from the site of generation of metabolites. The present disclosure provides that protecting the adsorbent from such binding in the upper GIT should allow for co-administration of drugs and nutrients, while diminishing drug-drug or drug-nutrient interactions. Further, materials and methods of the present disclosure can make the adsorbent more available for binding the metabolites, thus, increasing the binding efficacy of said adsorbent or adsorbents.

Accordingly, the present disclosure provides compositions and methods for the targeting of adsorbents to different parts of the lower GI. The present disclosure further provides targeting therapeutics to a site or sites of proteolytic enzyme activity (e.g., the distal colon).

Furthermore, the present disclosure provides compositions and methods for binding unabsorbed nutrients from the ileal effluent; binding small or large molecules in the colon; or therapeutics to specifically target the site of proteolytic enzyme activity.

Additionally, an object of the present disclosure is to provide pharmaceutical preparations yielding dosing regimens that are acceptable for taking other drugs simultaneously; reduce or minimizing dosing amounts/quantities administered, and increase efficacy compared to those achieved by conventional spherical carbon adsorbents, devoid of a coating formulation.

The present disclosure provides compositions and methods that serve as alternatives to immediate-release formulations which are encumbered and limited by their exposure of the adsorbents to the contents of the entire GIT, resulting in a much lowered or diminished efficacy/efficiency of adsorption of adsorbates.

The present disclosure provides compositions and methods related to the adsorption performance of conventional spherical carbon adsorbent after oral administration directed towards improving adsorption performance resulting from traveling through the GI-tract. The present disclosure provides compositions and methods to improve or mitigate the observation that when spherical carbon adsorbent is exposed to the intestinal lumen fluid, the ability of said spherical carbon adsorbent to adsorb uremic toxins or bacterial metabolites, or their precursors is reduced, and when said spherical carbon adsorbent is orally administered, the adsorption performance of said spherical carbon adsorbent is reduced at a stage before said spherical carbon adsorbent reaches the target caecum/large intestine, where intestinal bacteria are present and where uremic toxins or bacterial metabolites, or their precursors are adsorbed (i.e., at the duodenum/small intestine stage). Accordingly, the present disclosure provides evaluations of systematic design variations/physical modifications on a therapeutic agent (for example, a porous adsorbent material) directed toward maintaining optimal adsorption performance in the caecum/large intestine after oral administration, and further, the present disclosure provides pharmaceutical preparations and methods for preparing a pharmaceutical preparation that is capable of maintaining adsorption performance also in the caecum/large intestine while not affecting the plasma concentration of other drugs, when other drugs are simultaneously administered, as accomplished in the present disclosure.

The present disclosure provides a pharmaceutical preparation for delivery to the large intestine of a therapeutic agent, which enables a concomitant drug to be simultaneously administered, has a higher toxic-substance adsorbability than conventional therapeutic agents, and is effective for reducing blood uremic toxins, adsorbing bacterial metabolites, improving uremic symptoms, delaying dialysis initiation, or protecting renal functions in a CKD subject, by controlling the release of a therapeutic from a capsule preparation and/or the coated state of the toxic substance adsorbing surface of the therapeutic in the GI tract.

The pharmaceutical composition for oral administration preparations of the present disclosure act to adsorb uremic toxins or bacterial metabolites with greater efficacy or in larger quantities than conventional, non-coated spherical carbon adsorbents, and therefore are useful for reducing blood uremic toxins or bacterial metabolites, improving irritability in ASD, for treating, inhibiting or ameliorating behavioral symptoms of a neurological disorder such as autism or autism spectrum disorder (ASD), and associated pathologies including intestinal hyperpermeability or leaky gut, improving uremic symptoms, delaying dialysis initiation, or protecting renal functions in a subject with a renal disease, in particular, CKD. Examples of uremic symptoms of a subject with CKD include fatigue, anorexia, insomnia, pruritus, and nausea. It is known that some uremic toxins or bacterial metabolites, such as p-cresol sulfate originating from p-cresol produced by intestinal bacteria in the caecum/large intestine or descending colon, actively deteriorate renal functions. By adsorbing such uremic toxins or bacterial metabolites in the lumen of the GI tract to reduce the amount of uremic toxins or bacterial metabolites absorbed into the body, the pharmaceutical preparations of the present disclosure can protect renal functions and suppress progression of renal dysfunction (decrease in eGFR and increase in serum creatinine level and BUN) of a subject with CKD and delay transition to dialysis. It is expected that reduction of blood uremic toxins or bacterial metabolites improves vascular endothelial functions and suppresses calcification, and moreover it is expected that increasing the response of C.E.R.A. (Continuous EPO Receptor Activator) can contribute to treating or preventing anemia.

Without being limited by theory, in some embodiments for water insoluble therapeutic agents, the therapeutic agent, before being provided with a means for exposing its surface for the first time in the colon, has a specific surface area (a BET multipoint method) of 500 or more, to about 4000 (in some embodiments, 1000 to 1700) square meters per gram (m²/g) and an average particle size (determinable by a variety of techniques, including via laser diffractometry among others) of 0.005 mm to 1.5 mm. In some embodiments, the therapeutic agent has a specific surface area (a BET multipoint method) of less than 4000 m²/g, with some embodiments in the range of 1200 to 1600 (m²/g). The specific surface area (determined using a BET multipoint method) and the average particle size (for example, as determined by laser diffractometry) can be measured in accordance with the United States Pharmacopeia (USP) or the Japanese Pharmacopoeia.

Examples of therapeutic agents usable in the present disclosure include activated carbon (including spherical carbon adsorbents) and zeolites. In general, therapeutic agents are functional substances. For example, zeolites, which are sometimes called “boiling stones”, can be used for deodorization, moisture absorption, water absorption, water purification, and the like, and are used as a catalysts and separation materials in the petrochemical field. Activated carbon is used in purification and deodorization of water in the environmental field. In addition to zeolites and activated carbon, it is expected in recent years that PCP (porous coordination polymers)/MOF (metal-organic frameworks) can serve as novel therapeutic agents, and are applied to, for example, selective storage and sustained release of molecules and ions. In the present disclosure, the function of a therapeutic agent means, in particular, a material being capable of demonstrating the ability to adsorb target adsorbates (such as bacterial metabolites in the case of ASD; uremic toxins, bacterial metabolites, phosphorus in the case of hyperphosphatemia, and potassium in the case of hyperkalemia) under physiological conditions, as an oral adsorbent that can ensure efficacy as an orally-administered pharmaceutical product. The present disclosure is applicable to therapeutic agents displaying local (colonic) or systemic (in other parts of a subject living organism) therapeutic effects.

In some embodiments, the therapeutic agents of the present disclosure are commercially available, known, or produced by known production methods, which can be utilized herein. For example, activated carbon as one embodiment of the therapeutic agent, can be produced by carbonizing and activating a spherical phenol resin through heat treatment under predetermined conditions; washing and reheating the resin by predetermined methods, and sieving the resin as necessary, as indicated in Japanese Patent No. 3585043. The physicochemical properties of the therapeutic agent used in the present disclosure can be specified primarily by the specific surface area, the pore volume, and the packing density. For example, Japanese Patent No. 3585043 shows characteristics with values such as specific surface area: 800 to 2000 m²/g; pore volume: 0.2 to 1.0 mL/g; packing density: 0.5 to 0.75 g/m; pore diameter: 1.7 to 2.0 nm, maximum particle size: 425 mM or less, and average particle size: 350 mM or less. Japanese Patent No. 5124094 shows characteristics values such as specific surface area: 1150 to 1500 m²/g; pore volume: 0.2 to 1.0 mL/g; packing density: 0.5 to 0.7 g/mL, maximum particle size: 425 mM or less, and average particle size: 350 mM or less (Japanese Patent No. 5124094). Examples of other therapeutic agents for use in the present disclosure include medicinal carbon set forth in the United States Pharmacopoeia (USP), rice husk carbon or rice straw carbon containing amorphous silica (Japanese Patent, Laid-Open No. 2014-181144), organic porous bodies (Japanese Patent, Laid-Open No. 2014-77138), and PCP/MOF (Hirayasu Furukawa et al., Science 2010; 329: 424-428, Alexandra M. Z. Slawin et al., Angew. Chem. Int. Ed. 2010; 49: 8630-8634). Network/porous polymers, resins, and the like, used as therapeutic agents for hyperphosphatemia and hyperkalemia, can be used as well.

2. Adsorbents in Combination with Other Drugs as Therapeutic Agents to Adsorb One or More Toxins or Metabolites in the GI Tract

In some embodiments, the present disclosure provides for delivery of a suitably coated therapeutic agent core (e.g., activated carbon particles) that is intended to bind bacterial metabolites generated in the colon. In certain other embodiments, the present disclosure provides materials and methods to bind curli proteins and/or curli protein sub-units or fragments that Curli-producing bacteria residing in the colon, particularly the descending colon, can produce. In certain other embodiments, the present disclosure provides materials and methods suitable for co-administering an adsorbent with an amyloid inhibitor.

In other embodiments, an adsorbent of the present disclosure is co-administered with a modulator of secondary bile acid metabolism, and/or a secondary bile acid sequestrant (BAS). In some embodiments, an amyloid inhibitor is coated for targeted colonic release. In some embodiments, a modulator of secondary bile acid metabolism is coated for targeted colonic release. In other embodiments, a bile acid sequestrant (BAS) is coated for targeted colonic release.

### 3. Bile Acid Sequestrants as Therapeutic Agents to Adsorb One or More Toxins or Metabolites in the GI Tract

Bacterial gut microbes in the small intestine and colon metabolize conjugated primary bile acids to produce unconjugated primary and secondary bile acids. Increased levels of secondary bile acids in the colon can increase the risk of colon damage including inflammation, increased gastrointestinal permeability, and cancer. Increased levels of secondary bile acids in the liver via portal vein recirculation can increase the risk of liver damage including inflammation, non-alcoholic steato-hepatitis (NASH), fibrosis, cirrhosis, and cancer. Elevated secondary bile acids can cause inflammatory and immunological responses that extend beyond the gut to the periphery and the central nervous system. A cationic polymer molecule that selectively binds secondary bile acids such as deoxycholic acid (DCA) and lithocholic acid (LCA) and their conjugated forms in the GI could prevent colon damage, the reabsorption of secondary bile acids to the liver, and inflammatory and immunological sequelae.

A therapeutic with this profile could be used treat diseases related to secondary bile acids like colon cancer, Crohn's Disease, other inflammatory bowel diseases, maladies of the liver such as NASH, liver cancer (e.g., hepatocellular carcinoma (HCC)), fibrosis, and cirrhosis, Parkinson's disease, Alzheimer's disease and autoimmune diseases as well as modulate immune responses. A targeted approach, in which synthetic polymers selectively bind secondary bile acids over primary bile acids in the colon, can reduce or avoid upregulation of bile acid synthesis as has been seen with unselective bile acid sequestrants. A compound of this type that does not bind other acidic metabolites would also have an advantage over unselective binders. An additional advantage of using a polymeric bile acid sequestrant is the lack of systemic exposure which should provide a favorable safety profile.

In other embodiments, an adsorbent of the present disclosure is co-administered with a modulator of secondary bile acid metabolism, and/or a secondary bile acid sequestrant (BAS). In some embodiments, an amyloid inhibitor is coated for targeted colonic release. In some embodiments, a modulator of secondary bile acid metabolism is coated for targeted colonic release. In other embodiments, a bile acid sequestrant (BAS) is coated for targeted colonic release.

### Combination Therapies

In some embodiments, the pharmaceutical compositions for oral administration preparations of the present disclosure can be formed into a combination therapeutic agent or drug with the above-described therapeutic agents or drugs to be concomitantly-administered, dosed or used.

For example, a combination therapeutic agent or drug can be produced by encapsulating a therapeutic agent or drug coated with a colonic delivery formulation and a concomitant therapeutic agent or drug within one capsule. A combination therapeutic agent or drug can also be produced by encapsulating a therapeutic agent within a colon-targeted polymer layer or capsule and further encapsulating the colon-targeted polymer layer coated construct or capsule and a concomitant therapeutic agent or drug within a single capsule. The proportion of a concomitant therapeutic agent or drug to be combined can be variously set; in some embodiments, the ratio of a first therapeutic agent to a second therapeutic agent to be concomitantly used can be in the range of about 1:0.0001 to about 20; in other embodiments, the range can be about 1:0.0005 to about 10 in terms of weight ratio.

When used in combination with a further or additional therapeutic agent or drug, the pharmaceutical composition for oral administration preparation of the present disclosure can be administered concomitantly or simultaneously. Examples of usable concomitant therapeutic agents or drugs include a variety of oral therapeutic agents or drugs such: as stimulants, as methylphenidate, dexmethylphenidate, Adaphen, Artige, Cognil, Equasym, Inspiral, Methylin, Phenida, Prohiper, Tradea, Concerta, Concerta XL, Daytrana, Metadate CD, Equasym XL, Ritalin LA Ritalin SR, Rubifen SR, Penid, Focalin, Attenade, Quillivant XR, QuilliChew ER, Medikinet XL; Bupropion, Escitalopram, Thorazine; citalopram, sertraline, paroxetine, fluoxetine, Abilify, aripiprazole, Risperdal, risperidone, Brintellix, Trintellix, vortioxetine, sleep aids such as melatonin or melatonin receptor agonists; from biogenic amine reuptake inhibitors, biogenic amine transporter (BAT) inhibitors, selective serotonin reuptake inhibitors (SSRIs), dual norepinephrine-dopamine reuptake inhibitors (NDRIs), norepinephrine reuptake inhibitors (NERIs), dual serotonin-norepinephrine reuptake inhibitors (SNRIs), dopamine transporter (DAT) inhibitors, dopamine reuptake inhibitors, norepinephrine transporter (NET) inhibitors, serotonin modulators, serotonin receptor agonists, serotonin receptor partial-agonists, serotonin receptor inverse-agonists, serotonin receptor antagonists, muscarinic receptor agonists, muscarinic receptor antagonists, muscarinic receptor allosteric modulators, excitatory amino acid receptor modulators, AMPA receptor modulators, glutamate receptor modulators, metabotropic glutamate receptor modulators, ionotropic glutamate receptor modulators, antipsychotic agents, atypical antipsychotic agents, anti-insomnia agents, tricyclic antidepressants (TCAs), benzodiazepines, tranquilizers, hypnotics, sedatives, sedative-hypnotics, beta-adrenergic receptor blockers, cognition enhancers, nootropic agents, selective dopamine agonists, non-selective dopamine agonists (pan-agonists), dopamine receptor partial agonists, dopamine receptor positive allosteric modulators (PAMs), MAO inhibitors, angiotensin II receptor blockers, angiotensin converting enzyme inhibitors, calcium antagonists, diuretics, and antiplatelet drugs/anticoagulants, angiotensin II receptor blockers, angiotensin converting enzyme inhibitors, calcium antagonists, diuretics, hyperuricemia drugs, hyperlipidemia drugs, diabetes drugs, steroid/immunosuppressants, antiplatelet drugs/anticoagulants, hyperphosphatemia drugs, erythropoietic stimulating agents, analgesics, antiarrhythmic drugs, antidepressants, Attention Deficit Disorder (ADD) drugs, Attention Deficit Hyperactivity Disorder (ADHD) drugs, Alzheimer-type dementia drugs, Parkinson's disease drugs, proton pump inhibitors (PPIs), antiallergic drugs, and antibacterial drugs, sleep aids, cardiovascular drugs such as angiotensin II receptor blockers, angiotensin converting enzyme inhibitors, calcium antagonists, and diuretics used in drug therapy for subjects with chronic kidney disease; hyperuricemia drugs, hyperlipidemia drugs, diabetes drugs, steroids/immunosuppressants, antiplatelet drugs/anticoagulants, hyperphosphatemia drugs, erythropoietic stimulating agents, analgesics, antiarrhythmic drugs, antidepressants, Alzheimer-type dementia drugs, Parkinson's disease drugs, proton pump inhibitors (PPIs), antiallergic drugs, and antibacterial drugs prescribed based on the complications and primary diseases; and over-the-counter (OTC) drugs or therapeutic agents”

“Angiotensin II receptor blockers” refer to losartan, candesartan, valsartan, telmisartan, olmesartan, irbesartan, azilsartan, and the like”

“Angiotensin converting enzyme inhibitors” refer to captopril, enalapril, alacepril, derapril, cilazapril, lisinopril, benazepril, imidapril, temocapryl, quinapril, trandolapril, perindopril erbumine, and the like.

“Calcium antagonists” refer to nifedipine, amlodipine, efonidipine, cilnidipine, nicardipine, nisoldipine, nitrendipine, nilvadipine, barnidipine, felodipine, benidipine, manidipine, azelnidipine, alanidipine, diltiazem, and the like.

“Diuretics” refer to trichlormethiazide, benzylhydrochlorothiazide, hydrochlorothiazide, methiclane, indavamide, tripamide, mefruside, furosemide, triamterene, and the like.

“Antiplatelet drugs/anticoagulants” refer to aspirin, clopidogrel, prasugrel, ticlopidine, cilostazol, ethyl icosapentate, dipyridamole, sarpogrelate, beraprost, limaprost alfadex, warfarin, dabigatran, rivaroxaban, apixaban, edoxaban, rivaroxaban, apixaban, and the like.

### Treatment Methods

The dosages of the pharmaceutical composition for oral administration preparations of the present disclosure vary according to the symptoms, age, body weight, and the like, of the subjects in need thereof. Some embodiments are administered one to several times per day; some embodiments constitute about 300 mg to about 2000 mg in total daily dose of a therapeutic agent; some embodiments constitute about 300 mg to about 1000 mg dose at a time per adult subject, in terms of the amount of the therapeutic agent; and some embodiments constitute about 900 mg to 6000 mg or about 900 mg to 3000 mg as a daily dose.

### EXAMPLES

Without being limited by theory, some embodiments of the present disclosure are described in detail by way of the following examples. The examples of the present disclosure are not meant to be limiting, as the skilled artisan can appreciate numerous additional embodiments can be contemplated.

### Example 1

### Safety and Target-Engagement of an Oral Small Molecule Sequestrant in Adolescents with Autism Spectrum Disorder: An Open-Label Phase 1b/2a Trial

### Preclinical Methods

### Mouse Husbandry

All animal husbandry and experiments were approved by the Caltech Institutional Animal Care and Use Committee. Throughout the study, colonized animals were maintained in autoclaved microisolator cages with autoclaved bedding (Aspen Chip Bedding, Northeastern Products Corp, Warrensburg, NY), water, and chow. Standard chow was provided to the animals (Laboratory Autoclavable Rodent Diet—5010, LabDiet; St. Louis, MO, USA) until diet was switched to irradiated 5% AST-120 (Kureha Corp., Tokyo, JP) or control diets (Teklad). This percentage of AST-120 in mouse chow was previously used safely in mice⁸⁶. Mice were maintained at an ambient temperature of 71-75F, 30%-70% humidity, at a cycle of 13 hours light and 11 hours dark.

### Experimental Design of Mouse Experiments

Germ-free (GF) C57BL/6J male weanlings (3 weeks of age) from the Mazmanian laboratory colony (CalTech) were colonized by gavage of 100 ul of 1:1 mixture of 10⁹ CFU/ml B. ovatus (+/−4EP pathway genes) and wild type L. plantarum. At 5 weeks of age, mice were switched to the irradiated 5% AST-120 or control diets (Teklad) for the remainder of the experiment. Mice were weighed weekly beginning at diet switch. Urine was collected at 7 weeks of age prior to behavior testing. Behavior testing began at 7 weeks of age, 3 days after urine collection.

Analysis of Metabolites from Urine of Mice

Urine was passively collected by brief restrain of mouse over aluminum foil. 4EPS levels were quantified by LC/MS and normalized to creatinine levels by Charles River Laboratories (Boston, MA).

### Behavior Testing

Behavior testing was performed as previously described^{34,87,88}. All mice were tested using the same battery of behavioral tests, starting at six weeks of age, in the following order: EPM, open-field testing, marble burying, grooming, social behavior, and USV (male-female context). Mice were allowed to settle for at least two days after cage changing before they were tested, and tests were performed 2-3 days apart to allow mice to rest between tests. Mice were acclimated to the behavior testing room for one hour prior to testing. Mice were tested during the light phase of the light/dark cycle.

### Elevated Plus Maze (EPM)

EPM was performed in a maze with 25 cm by 5 cm arms and a 5 cm by 5 cm center, recorded using an overhead camera, and tracked and analyzed using the EthoVision XT 10 software package (Noldus Information Technology; Leesburg, VA, USA). Prior to testing, the maze was disinfected using Rescue disinfectant (Virox technologies, Oakville, ON, Canada) then allowed to evaporate. Mice were then introduced to the arena and allowed to explore for 5 minutes while being tracked. The number of entries into and the time spent in open and closed arms as well as the outer third of the open arms (the terminus) were analyzed. If a mouse fell or jumped from the apparatus during the test it was removed from the dataset.

### Open-Field Test

The open-field test was performed in 50×50 cm² white Plexiglas arenas, recorded using an overhead camera, and tracked and analyzed using the EthoVision XT 10 software package (Noldus Information Technology; Leesburg, VA, USA). Prior to testing, the arena was disinfected using Rescue disinfectant (Virox technologies, Oakville, ON, Canada) then allowed to evaporate. Mice were then introduced to the arena and allowed to explore for 10 minutes while being tracked. The total distance traveled, and the number of entries and time spent in a 17×17 cm² center square were analyzed. Fecal pellets left during the assay were quantified.

### Marble Burying

Marble burying was performed in a normal cage bottom (Lab Products; Seaford, DE) filled with 3-4 cm of fresh, autoclaved wood chip bedding (Aspen chip bedding, Northeastern Products Corp; Warrensburg, NY). Mice were first habituated to the cage for 10 minutes, and subsequently transferred to a holding cage while the bedding was leveled, and 20 glass marbles (4×5) were placed on top. Mice were then returned to their own cage and removed after 10 minutes. The number of buried marbles (50% or more covered) was then recorded and photographed for reference. A fresh cage was used for each mouse, and marbles were soaked in Rescue disinfectant (Virox technologies, Oakville, ON, Canada) and dried in bedding in between tests.

### Grooming

Mice were placed in autoclaved, empty standard cages (Lab Products; Seaford, DE) and video recorded from the side for 15 minutes. The final 10 minutes were scored manually by a blinded, trained researcher for grooming behavior.

### Clinical Methods

### Clinical Study Design and Ethical Approval

The AXL-2004-001 study (ANZCTR (anzctr.org.au/) ACTRN12618001956291) was an open-label, outpatient, multiple ascending dose Phase 1b/2a study in an ASD-diagnosed adolescent (12-<18 years old) population with confirmed gastrointestinal symptoms (e.g., diarrhea, constipation, abdominal pain, bloating). 41 individuals were screened between Apr. 18, 2019 and Jan. 23, 2020. 30 participants were enrolled across three sites in Australia and New Zealand, including the Queensland Children's Hospital in Brisbane (14 subjects), Brain and Mind Centre in Sydney (6 subjects), and Optimal Clinical Trials in Auckland (10 subjects). There was no formal sample size calculation for the phase I study because it focused on safety and tolerability. This approach was common in early-stage exploratory clinical trials. All necessary licenses and permissions to use the behavioral assessments outlined in the study protocol were obtained prior to initiating the study.

The study protocol, investigator brochure, participant information and consent forms, participant facing questionnaires, recruitment documentation and procedures, and documentation regarding the investigators experience and qualifications were submitted to Health and Disability Ethics Committees (New Zealand), Children's Health Queensland Hospital and Health Service Human Research Ethics Committee, and Bellberry Human Research Ethics Committee for ethical review and approval. The study was conducted in accordance with the Declaration of Helsinki (Fortaleza, October 2013), ICH E6 guidelines, good clinical practices, and local regulations.

### Study Participation

This open label study consisted of 4 different dosing plans based on the subject's weight at Visit 1 (V1). Eligible subjects were escalated through three dosing periods during the 8-week treatment period starting with the lowest dose for their dosing plan. See Supplemental Methods for more details. Subjects were requested to consume AST-120 90 minutes after any other concomitant medications. Safety and tolerability were confirmed before a subject escalated to the next dosing level. If subjects were unable to tolerate a dosing level, they were returned to previous dosing level for the remainder of the treatment period. Following the last dose of AST-120 subjects returned to the clinic 28 days later for a follow-up safety evaluation (Final Visit, FV). The last visit of the study was completed on May 15, 2020.

### Study Participants and Study Populations

A total of 41 adolescent subjects, aged 12-17 years inclusive, were screened for eligibility for participation in the study, and the 30 who met the study-specific eligibility criteria were enrolled and received at least one dose of AST-120 (Safety Population). Of the 41 subjects screened and 30 enrolled, 40 and 29, respectively, were male. A predominantly male cohort was targeted to reduce variability in response in this exploratory study that surveyed a wide range of behavioral assessments. One participant withdrew after the first dose due to the investigator's decision based on the subject presenting with an unrelated viral infection. Another subject withdrew consent during the low dose period due to anticipated admission to hospital for pre-existing behavioral difficulties. One participant withdrew due to significant study non-compliance, and two did not complete FV assessments due to the caregiver being unwell and unable to accompany the subjects. A total of 27, (26 male and 1 female), completed at least up to the End of Treatment (EOT) visit (Completers Population). One subject, the female participant, was included in the Safety Population, but was not included in the exploratory efficacy analysis. This subject was removed from the exploratory efficacy analysis because their participation in the trial coincided with the initial COVID-19 pandemic outbreak and its associated societal restrictions put into effect in Australia. These restrictions prevented the subject from conducting normal routines and accessing normal services. As determined by the site Principal Investigator, these abrupt changes in routine had an impact on the behavior of the participant; therefore, this subject was excluded from the efficacy analysis.

### Safety Assessments

The primary endpoint of the study was the safety and tolerability of AST-120 as assessed by physical exams, vital signs, clinical laboratory measurements (hematology, serum chemistry, urinalysis), and Adverse Events.

### Blood Collection

Blood was obtained using uniform collection kits from Sonic Clinical Trials (SCT)(Australia) sent to each facility. Blood was drawn from study participants on visits 1, 4, and 5 and aliquoted for health monitoring by Sonic Clinical Trials (SCT) (Australia) and metabolite analysis by Metabolon, Inc (Durham, NC). Blood chemistry panels performed by SCT included albumin, alkaline phosphatase, alanine amino transferase, aspartate amino transferase, blood urea nitrogen, urea, corrected calcium, bicarbonate, chloride, creatinine, gamma-glutamyl transpeptidase, glucose, lactate dehydrogenase, magnesium, phosphorus, potassium, sodium, total bilirubin, conjugated bilirubin, unconjugated bilirubin, and total protein. Haematology panels included measurement of platelets, haematocrit, red blood cells, haemoglobin, reticulocytes, total white blood cell count and absolute and percentages of neutrophils, lymphocytes, monocytes, eosinophils, and basophils.

### Urine Collection

Each participant was provided with a urine home collection kit and instructions to collect all of the first morning void a maximum of 2 days before clinic visit and place in refrigerator to bring to their visit or to be picked up by courier. Urinalysis samples were collected during the in-clinic visit. Aliquoting for metabolite analysis and health monitoring urinalysis were performed by SCT and included measurements of pH, specific gravity, ketones, protein, glucose, nitrite, urobilinogen, leukocyte esterase, and blood.

### Human Plasma Metabolite Quantification

Human plasma was analyzed by Metabolon, Inc (Durham, NC). Briefly, plasma was spiked with internal standards (4-ethylphenyl sulfate-d₄, p-cresol sulfate-d₇, 3-hydroxyhippurate-¹³C₂, ¹⁵N, 3-hydroxyphenylacetate-d₃, 3-(3-hydroxyphenyl)-3-hydroxypropionate-d₃, 3-indoxyl sulfate-¹³C₆, 3-(4-hydroxyphenyl)propionate-d₄, p-cresol glucuronide-d₇, N-acetylserine-d₃,), protein precipitated, and analyzed on an Agilent 1290/AB Sciex 5500 QTrap LC-MS/MS system equipped with a UHPLC C18 column. Quantitation was performed using a weighted linear least squares regression analysis with a weighting of 1/x or 1/x² generated from fortified calibration standards prepared immediately prior to each run.

### Human Urine Metabolite Quantification

Human urine was analyzed by Metabolon, Inc (Durham, NC). Briefly, urine was diluted 10-fold and spiked with internal standards (p-cresol sulfate-d₇, 3-hydroxyhippurate-¹³C₂, ¹⁵N, 3-hydroxyphenylacetate-d₃, 3-(3-hydroxyphenyl)-3-hydroxypropionate-d₃, 3-indoxyl sulfate-¹³C₆, 3-(4-hydroxyphenyl)propionate-d₄, and p-cresol glucuronide-d₇, N-acetylserine-d₃,), then an aliquot was subjected to either a solvent crash (for p-cresol sulfate, 3-indoxyl sulfate, and p-cresol glucuronide) or derivatization (for 3-hydroxyhippurate, 3-hydroxyphenylacetate, 3-(3-hydroxyphenyl)-3-hydroxypropionate, N-acetylserine, and 3-(4-hydroxyphenyl)propionate) and analyzed on an Agilent 1290/AB Sciex 5500 QTrap LC-MS/MS system equipped with a UHPLC C18 column in negative mode. Quantification of 4-ethylphenyl sulfate was performed by the same method with a solvent crash (using the internal standard, 4-ethylphenyl sulfate-d₄), but without sample dilution. Quantitation was performed using a weighted linear least squares regression analysis with a weighting of 1/x generated from fortified calibration standards prepared immediately prior to each run. All urine metabolites were normalized to creatinine levels.

### Exploratory Efficacy Assessments

Exploratory efficacy outcomes included changes from BL at EOT and FV on the GSI-6, NRS, GSRS, BSS, RBS-R VABS, CASI-5, SRS, CGI-S and CGI-I, ABC, or PARS diagnostics. Efficacy assessments were administered on site at the respective clinics during visits. VABS, PARS, and CGI-S and CGI-I were conducted by the PI or qualified designee. The GSI-6, NRS, GSRS, RBS-R, BSS, CASI-5 SRS, and ABC questionnaires were completed by the designated caregivers of the participants. In the VABS assessment, 10 participants did not pass the under 25% estimated answers criterion of any domain during assessment, and thus had to be removed from this analysis, according to the VABS manual, p. 47.5

### AST-120 Treatment Dosage

For subjects weighing ≥60 kgs, three daily doses each of:
Period 1:0.75 g Days 1-14 (2 weeks)
Period 2: 1.5 gm Days 15-28 (2 weeks)
Period 3: 2 g Days 29-56 (4 weeks)

For subjects weighing 50-<60 kgs, three daily doses each of:
Period 1:0.75 g Days 1-14 (2 weeks)
Period 2: 1.0 g Days 15-28 (2 weeks)
Period 3: 1.75 g Days 29-56 (4 weeks)

For subjects weighing 40-<50 kgs, three daily doses each of:
Period 1:0.5 g Days 1-14 (2 weeks)
Period 2:0.75 g Days 15-28 (2 weeks)
Period 3: 1.5 g Days 29-56 (4 weeks)

For subjects weighing 30-<40 kgs, three daily doses each of:
Period 1:0.5 g Days 1-14 (2 weeks)
Period 2:0.75 g Days 15-28 (2 weeks)
Period 3: 1.0 g Days 29-56 (4 weeks)

### Magnetic Resonance

### MR Scan Protocol

The imaging protocol was designed with a number of competing requirements. To meet the goals of the study, the protocol was designed first, to obtain anatomic, resting state, and diffusion weighted image data of high quality, second, to be well tolerated by the target subjects, and third, to be comparable between the three imaging sites. The study design used each subject as their own control, to mitigate effects between sites and scanners. Prior to design the imaging sites were consulted, both to ascertain their system capabilities, and to draw on their extensive experience imaging subjects with ASD, before designing a protocol. The major design criteria were therefore:
1) The protocol must acquire high resolution anatomic images, rs-fMRI, and DTI data.
2) EPI based scans (rs-fMRI and DTI) would need to be distortion corrected.
3) Total scan time needed to be 45 minutes or less, and individual scans should be kept as close to 5 minute duration as possible, due to subject tolerance.
4) It was expected that any given scan might be corrupted by motion, so scans were designed to be partially redundant.
5) The scan parameters needed to be as consistent as possible between sites, despite different scanner manufacturers and models (Site 1: GE Discovery 750, software level DV26.0, Site 2: Siemens Skyra, software level VE11, Site 3: Siemens Prisma Fit, software level VE11).

### Scan Parameters

All scans were collected using phased array receive only head coils (32 channels at Sites 1 and 2, 64 channels at Site 3). High resolution anatomic images (T1w and T2w) were acquired with 1 mm isotropic resolution. T1w images (2@4:01 each, for sites 1 and 2, 1@4:00 for site 3) were sagittally oriented using a 3D MPRAGE sequence. A single resolution matched T2w image (4:28) was acquired (the T2_CUBE sequence at Site 1, T2_SPACE at Sites 2 and 3). Two gradient echo multiband EPI rs-FMRI acquisitions (300 volumes each) were performed with 2.5 mm isotropic resolution, 1 second repetition time, multiband factor 3. 51 slices were acquired obliquely with the bottom slice oriented on the line between the bottom of the cerebellum and the bottom of the orbitofrontal cortex. The phase encode was reversed between the first and second scan (AP for the first scan, PA for the second) to allow for distortion correction. Two diffusion scans were also acquired as part of the protocol (5:56 each), but they were not used for this analysis.

### Data Processing

Prior to processing, all data were named and organized following the BIDS 1.2.1 specification. Anatomical and fMRI data used in this manuscript were preprocessed using fMRIPrep 20.0.489,90 (RRID:SCR_016216), which is based on Nipype 1.4.291 (RRID:SCR_002502).

### Anatomical Data Preprocessing

A total of 2 T1-weighted (T1w) images were found within each BIDS dataset. All of them were corrected for intensity non-uniformity (INU) with N4BiasFieldCorrection, distributed with ANTs 2.2.0⁹³ (RRID:SCR_004757). The T1w-reference was then skull-stripped with a Nipype implementation of the antsBrainExtraction.shworkflow (from ANTs), using OASIS30ANTs as target template. Brain tissue segmentation of cerebrospinal fluid (CSF), white-matter (WM) and gray-matter (GM) was performed on the brain-extracted T1w using fast (FSL 5.0.9, RRID:SCR_002823). A T1w-reference map was computed after registration of 4 T1w images (after INU-correction) using mri_robust_template (FreeSurfer 6.0.1). Brain surfaces were reconstructed using recon-all (FreeSurfer 6.0.1, RRID:SCR_001847), and the brain mask estimated previously was refined with a custom variation of the method to reconcile ANTs-derived and FreeSurfer-derived segmentations of the cortical gray-matter of Mindboggle (RRID:SCR_002438). Volume-based spatial normalization to two standard spaces (MNI152NLin6Asym, MNI152NLin2009cAsym) was performed through nonlinear registration with antsRegistration (ANTs 2.2.0), using brain-extracted versions of both T1w reference and the T1w template. The following templates were selected for spatial normalization: FSL's MNI ICBM 152 non-linear 6th Generation Asymmetric Average Brain Stereotaxic Registration Model [RRID:SCR_002823; TemplateFlow ID: MNI152NLin6Asym], ICBM 152 Nonlinear Asymmetrical template version 2009c [RRID:SCR_008796; TemplateFlow ID: MNI152NLin2009cAsym].

### Functional Data Preprocessing

For each of the 4 BOLD runs found per subject (across all tasks and sessions), the following preprocessing was performed. First, a reference volume and its skull-stripped version were generated using a custom methodology of fMRIPrep. A deformation field to correct for susceptibility distortions was estimated based on fMRIPrep's fieldmap-less approach. The deformation field was that resulting from co-registering the BOLD reference to the same-subject T1w-reference with its intensity inverted. Registration was performed with antsRegistration (ANTs 2.2.0), and the process regularized by constraining deformation to be nonzero only along the phase-encoding direction, and modulated with an average fieldmap template. Based on the estimated susceptibility distortion, a corrected EPI (echo-planar imaging) reference was calculated for a more accurate co-registration with the anatomical reference. The BOLD reference was then co-registered to the T1w reference using bbregister (FreeSurfer) which implements boundary-based registration. Co-registration was configured with six degrees of freedom. Head-motion parameters with respect to the BOLD reference (transformation matrices, and six corresponding rotation and translation parameters) are estimated before any spatiotemporal filtering using mcflirt (FSL 5.0.9). BOLD runs were slice-time corrected using 3dTshift from AFNI 20160207 (RRID:SCR_005927). The BOLD time-series (including slice-timing correction when applied) were resampled onto their original, native space by applying a single, composite transform to correct for head-motion and susceptibility distortions. These resampled BOLD time-series were referred to as preprocessed BOLD in original space, or just preprocessed BOLD. The BOLD time-series were resampled into standard space, which generated a preprocessed BOLD run in MNI152NLin6Asym space. First, a reference volume and its skull-stripped version were generated using a custom methodology of fMRIPrep. Automatic removal of motion artifacts using independent component analysis (ICA-AROMA) was performed on the preprocessed BOLD on MNI space time-series after removal of non-steady state volumes and spatial smoothing with an isotropic, Gaussian kernel of 6 mm FWHM (full-width half-maximum). Corresponding “non-aggressively” denoised runs were produced after such smoothing. Additionally, the “aggressive” noise-regressors were collected and placed in the corresponding confounds file. Several confounding time-series were calculated based on the preprocessed BOLD: framewise displacement (FD), DVARS and three region-wise global signals. FD and DVARS were calculated for each functional run, both using their implementations in Nipype (following the definitions by Power et al. 2014). The three global signals were extracted within the CSF, the WM, and the whole-brain masks. Additionally, a set of physiological regressors were extracted to allow for component-based noise correction (CompCor). Principal components were estimated after high-pass filtering the preprocessed BOLD time-series (using a discrete cosine filter with 128 s cut-off) for the two CompCor variants: temporal (tCompCor) and anatomical (aCompCor). tCompCor components were then calculated from the top 5% variable voxels within a mask covering the subcortical regions. This subcortical mask was obtained by heavily eroding the brain mask, which ensured it did not include cortical GM regions. For aCompCor, components were calculated within the intersection of the aforementioned mask and the union of CSF and WM masks calculated in T1w space, after their projection to the native space of each functional run (using the inverse BOLD-to-T1w transformation). Components were also calculated separately within the WM and CSF masks. For each CompCor decomposition, the k components with the largest singular values were retained, such that the retained components' time series were sufficient to explain 50 percent of variance across the nuisance mask (CSF, WM, combined, or temporal). The remaining components were dropped from consideration. The head-motion estimates calculated in the correction step were also placed within the corresponding confounds file. The confound time series derived from head motion estimates and global signals were expanded with the inclusion of temporal derivatives and quadratic terms for each. Frames that exceeded a threshold of 0.5 mm FD or 1.5 standardised DVARS were annotated as motion outliers. All resamplings were performed with a single interpolation step by composing all the pertinent transformations (i.e., head-motion transform matrices, susceptibility distortion correction when available, and co-registrations to anatomical and output spaces). Gridded (volumetric) resamplings were performed using antsApplyTransforms (ANTs), configured with Lanczos interpolation to minimize the smoothing effects of other kernels. Non-gridded (surface) resamplings were performed using mri_vol2surf (FreeSurfer). Many internal operations of fMRIPrep use Nilearn 0.6.2 (RRID:SCR_001362), mostly within the functional processing workflow.

fMRI Data Analysis

To quantify connectivity between the bilateral amygdala and rostral anterior cingulate cortex (rACC), a region of interest (ROI) approach was used employing methods from prior work. The bilateral amygdala was defined using the Harvard-Oxford atlas. The rACC ROI was just anterior to the genu of the corpus callosum and had been used in prior work. This ROI was defined by a 5 mm sphere located at Montreal Neurological Institute (MNI) coordinates x=0, y=38, z−4. Average time courses for each ROI were extracted, demeaned, detrended, Hamming windowed, and correlated to generate a single correlation value (r) for each participant both before and after treatment. Baseline and end of treatment values for amygdala-rACC coupling were compared using a paired t-test. The treatment-induced change in bilateral amygdala-rACC coupling was then correlated with baseline anxiety score.

### Statistical Information

Results presented here were from post hoc analyses of the data from the clinical trial using Graphpad Prism 9. Here, bar graphs representing the preclinical data by mean±SEM analyzed by ordinary two-way ANOVA test with FDR correction using the Benjamini Krieger and Yekutieli method, with individual variances computed for each comparison were presented. Clinical data was presented as mean and 95% confidence intervals analyzed by Repeated Measures ANOVA, or linear mixed effects model, with Geisser-Greenhouse correction tests and false discovery rate correction by the Benjamini, Krieger and Yekutieli method. Metabolite data was presented as individual graphs but was statistically analyzed across all metabolites and samples. Clinical behavioral metrics were analyzed within each test. Pearson's correlations were performed comparing change in metabolite levels to change in behavioral scores for the PARS and ABC-I tests. fMRI values were analyzed using a two-tailed paired t-test. Study participants were studied as a single group, and all comparisons, especially those within the subgroup of individuals in the top quartile of ASD severity were post hoc and exploratory in nature. Missing data were not imputed, and data were analyzed for subjects who withdrew from the study, for any reason prior to study completion, regardless of treatment duration, up to the point of discontinuation.

Autism spectrum disorder (ASD) is defined by hallmark behaviors involving reduced communication and social interaction, as well as repetitive activities and restricted interests. ASD represents a broad spectrum from minimally affected individuals to those requiring intense support, with additional manifestations often including anxiety, irritability/aggression, and altered sensory processing. Gastrointestinal (GI) issues are also common in ASD, and studies have identified changes in the gut microbiome of individuals with ASD compared to control populations, which complemented findings of differences in intestinal metabolites in feces and circulation. However, a role for the gastrointestinal tract or microbiome in ASD remained controversial. Herein, an oral gastrointestinal-restricted adsorbent (AST-120) was reported, that had affinity for small aromatic or phenolic molecules, relieved anxiety-like behaviors that were driven by a gut intestinal metabolite in mice. Accordingly, a pilot human study was designed and completed to evaluate the safety of AST-120 in an open-label, single cohort, multiple ascending dose clinical trial that enrolled 30 adolescents with ASD and gastrointestinal symptoms in New Zealand and Australia. AST-120 was shown to have good safety and tolerability across all dose levels, and no drug-related serious adverse events were identified. Significant reductions in specific urinary and plasma levels of gut bacterial metabolites were observed between baseline and end of ASY-120 treatment, demonstrating likely target engagement. Furthermore, improvements were observed in multiple exploratory behavioral endpoints, most significantly in post-hoc analysis of anxiety and irritability, as well as gastrointestinal health after 8 weeks of treatment. These results from an open-label study (trial registration #ACTRN12618001956291) suggested that targeting intestinal metabolites with an oral adsorbent was a safe and well-tolerated approach to improving symptoms associated with ASD and thereby emboldened larger placebo-controlled trials.

AST-120 is a high surface-area spherical carbon adsorbent that has affinity for uremic toxins including those of gut bacterial origin, such as the simple phenols, 4EPS, p-cresyl sulfate (pCS), and p-cresyl glucuronide (pCG), as well as the indole derivative, 3-indoxyl sulfate (3IS) and hippuric acid, based on evidence from rodent models and patients with chronic kidney disease and IBS. It was found that, taken orally, it binds and sequesters related aromatic metabolites as it passes through the gastrointestinal tract without being absorbed and was ultimately excreted, effectively lowering systemic metabolite exposure. It was hypothesized that AST-120 would also reduce the structurally related 3-hydroxyhippurate (HHA) and phenylpropanoic acids, or other related small-molecule metabolites such as 3-(3-hydroxyphenyl)-3-hydroxypropionate (HPHPA), 3-(4-hydroxyphenyl)propionate (HPPA), 3-hydroxyphenylacetate (HPAA), 3-carboxy-4-methyl-5-propyl-2-furanpropanoate (CMPF), and imidazolepropionate (IPA). There was accumulating evidence that increased levels of this chemical class of intestinal metabolites was associated with ASD. For instance, 4EPS, pCS, 3IS, hippuric acid, and hydroxyphenylacetic acid metabolite levels were found to be elevated in children with ASD, and levels of some of these metabolites also correlated with gastrointestinal and behavioral symptoms. These findings go beyond simple associations between intestinal metabolites and behavioral endpoints; namely production of 4EPS by gut bacteria resulted in changes in brain cell function and increased anxiety-like and ASD-like behaviors in mice. pCS administered to mice lead to deficits in social communication and repetitive behaviors, and both pCS and 3IS promoted anxiety-like and depression-like features in rodents. To date, no studies have attempted to modify the production or concentrations of this class of compound in human neuropsychiatric disorders.

### Results

### 2AST-120 Reduces 4EPS and Anxiety-Like Behavior in Mice.

It was reported that 4EPS was elevated in the plasma of individuals with ASD, though bacterial sources for production of the metabolite remained unknown. The gut microbiome is predicted to harbor genes that convert tyrosine, the precursor of several mammalian neurotransmitters, to 4-ethylphenol (4EP), which could then be sulfated to 4EPS. Sulfation in the liver or other organs is a common detoxifying activity in mice and humans for structurally related phenolic molecules.

Several bacterial species were systematically tested for the enzymatic activity required for biosynthesis of 4EP from tyrosine. Next, genes that showed predicted activity were cloned into genetically tractable strains of gut bacteria. Subsequently, gnotobiotic mice were colonized with isogenic bacterial strains that were engineered to convert tyrosine to 4-ethylphenol (4EP+group), or mutants of the same strains that lack genes encoding enzymes that mediate this conversion (4EP− group). It was verified that gut microbial production of 4EP, followed by efficient host sulfation, lead to the presence of 4EPS in urine of 4EP+ mice (FIGS. 4A-4C; control). 4EPS promoted anxiety-like behavior in several testing paradigms: first, open-field exploration where mice ventured less into the more exposed zone of the arena, second, the elevated plus maze (EPM) where 4EP+ mice spent less time in the terminus of the open arms, and third, the marble burying test (FIGS. 4F-I). This experimental paradigm enabled effective testing of drugs that neutralized 4EPS.

Following colonization, both groups of mice were placed on a diet consisting of 5% AST-120 by weight of chow or matched control diet two weeks prior to behavior testing (FIG. 4B). As expected, systemic levels of 4EPS measured in urine were lowered by oral AST-120 administration (FIG. 4C). There were no differences in bacterial colonization levels and weight gain was not significantly influenced (FIGS. 4D, 4E), and no signs of distress, illness or other differences were observed among the mouse groups. Behavioral analysis revealed that treatment with AST-120 ameliorated behavioral deficits in the open field and elevated plus maze tests for anxiety (FIGS. 4F-4H). Specifically, 4EP+ mice given AST-120 spent more time exploring the exposed (i.e., riskier) areas of the tests compared to 4EP+ mice on the control diet. AST-120 treatment also stabilized performance in the anxiety/repetitive behavior-related marble burying test (FIG. 4I). These results were accompanied by trending improvements in the grooming test for repetitive behavior (FIG. 4J). Results from this simplified mouse model indicated that AST-120 was effective in reducing systemic levels of 4EPS and prevented 4EPS-induced anxiety-like behaviors.

### Phase I Clinical Trial Design.

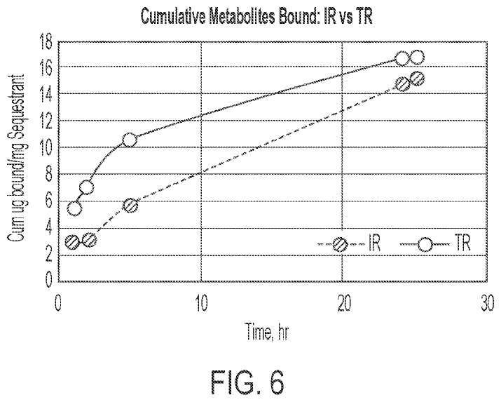

An open-label, phase 1b/2a clinical trial was designed and conducted at three sites in New Zealand and Australia with primary endpoints for safety and tolerability as determined by reported/observed adverse effects and laboratory results. Secondary endpoints included target engagement, which was assessed objectively by measuring microbially-derived metabolites in plasma and urine. Behavioral endpoints were exploratory. At screening, ASD diagnosis was confirmed using the Autism Diagnostic Observational Schedule, Second Edition (ADOS-2) and the presence of gastrointestinal symptoms was verified through the Gastrointestinal Severity Index (6-GSI) and a 14-day bowel habit diary. 30 adolescents (29 male, 1 female) previously diagnosed with ASD (FIG. 1A, FIG. 6A) met the inclusion criteria as outlined in Table 2. After enrollment, each participant was administered baseline behavioral assessments using the Pediatric Anxiety Rating Scale (PARS), Aberrant Behavior Checklist (ABC), Social Responsiveness Scale (SRS-2), Repetitive Behavior Scale Revised (RBS-R), and Vineland Adaptive Behavior Score (VABS-3) and several gastrointestinal symptom metrics were measured, including the 6-GSI53, Bristol Stool Scale (BSS), and Gastrointestinal Symptom Rating Scale tool (GSRS). All inclusion and exclusion criteria can be found in Table 2. Study participants were asked to take three daily, weight-adjusted (see Methods for details), ascending oral doses of AST-120 totaling ≤2.25 g/day, escalating to ≤4.5 g/day at 2 weeks and ≤6 g/day daily at 4 weeks until the end of treatment on Week 8, with a final visit 4 weeks after end of treatment (FIG. 1B). Urine, blood and stool samples were collected, and behavioral assessment performed, at baseline (BL), end of treatment (EOT), and final visit (FV), with continuous health monitoring throughout (FIG. 1B, Table 3). 27 of 30 enrolled participants completed to the EOT, but one was removed due to COVID schedule interruptions, resulting in a completers group of 26. 24 participants completed to the FV.

AB-2004 is Safe and Well Tolerated in Adolescents with ASD.

Assessment of overall health, including gastrointestinal symptoms, was determined by the clinical global impressions scale for severity and improvement (CGI-S and CGI-I, respectively). 76.9% of participants (20 out of 26) improved at least one point on the CGI-I scale from BL to EOT (FIG. 5B). While gastrointestinal symptoms were an inclusion criterion based on 6-GSI and a 14-day bowel habit e-diary assessment during screening, 19.2% of participants presented with no clinical gastrointestinal disorder based on the CGI at the time of assessment (including normal and borderline scores). Importantly, the number of participants with no measurable gastrointestinal symptoms doubled from BL to EOT (19.2% to 38.5%) (FIG. 5C).

Median adherence to dosing was 97.5% and no laboratory concerns arose, showing AST-120 was well tolerated. Importantly, overall safety metrics showed that no serious adverse events related to the drug or any deaths occurred during the reporting period of the study. The majority of mild or moderate adverse effects were in the gastrointestinal category, including abdominal pain and nausea (Table 1). The study therefore met its primary endpoints for safety and tolerability, extending the safety record of this drug to an adolescent ASD population for the first time.

### Microbial Metabolite Levels are Lowered by AB-2004.

Based on the known pharmacology of binding phenolic compounds, and its practically complete lack of systemic absorption, it was hypothesized that oral AST-120 would diminish levels of specific intestinal metabolites in circulation by facilitating their excretion in the feces. As predicted, AST-120 treatment resulted in reduced levels of 4EPS, pCG, pCS, 3IS, HPHPA, and HPAA in urine from the BL to EOT timepoints (FIGS. 2A-2G, FIG. 6A), with similar profiles in plasma (FIG. 6B). Concentrations between urine and plasma were highly correlated for many metabolites (FIG. 6C). Urine metabolites largely rebounded to pre-drug (BL) levels at the FV timepoint 4 weeks after treatment had concluded, which supported the conclusion that metabolite levels were influenced directly by AST-120 administration (FIGS. 2A-2G). N-acetyl serine levels, which were measured as a control metabolite not bound by AST-120, did not change in urine or plasma between BL and EOT (FIG. 6D). These data indicated that target engagement of gut-derived intestinal metabolites by AST-120 could effectively reduce their systemic levels.

### Oral AB-2004 May Alter Brain Connectivity.

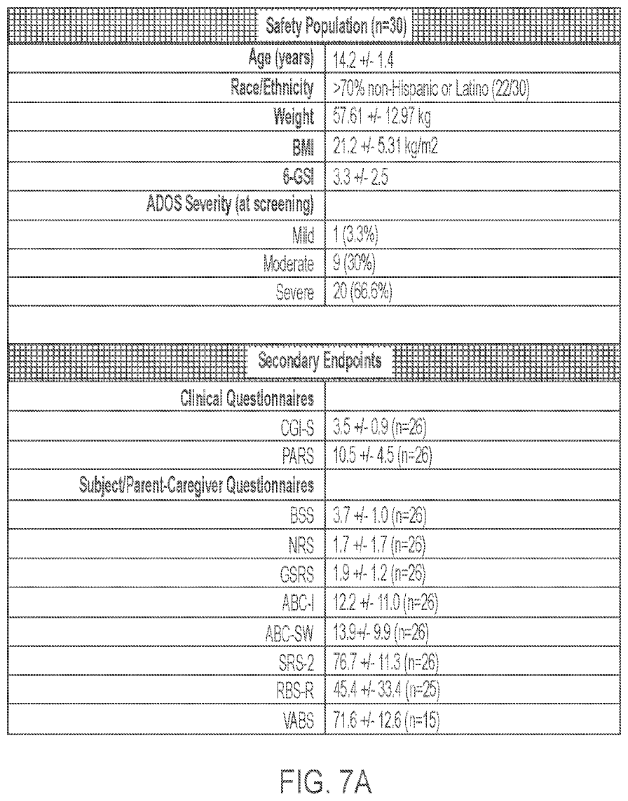

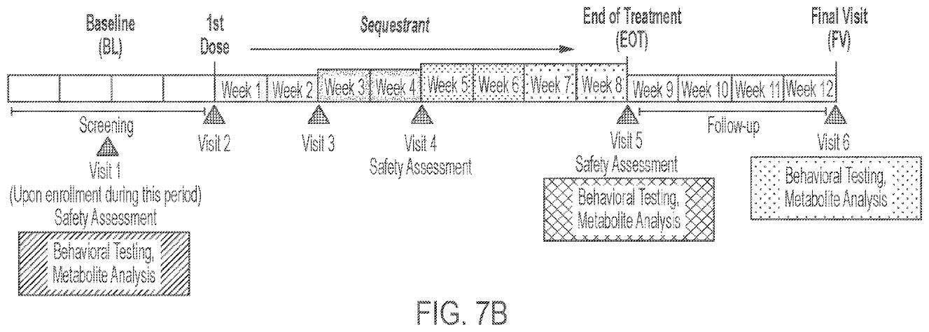

As a measure of brain activity patterns, resting state functional magnetic resonance imaging (fMRI) were performed on a small subset of ten study participants to estimate connectivity between brain regions. Two 5-minute scans were conducted at BL and EOT timepoints that focused on changes in regions associated with emotional behavior responses. This included regions such as the amygdala, which was crucial for emotional processing networks such as those involving anxiety, and the anterior cingulate cortex (ACC), which was involved in emotional and cognitive networks. Atypical activity in one or both of these regions was observed in preclinical studies⁶³ and in ASD cohorts. A decrease in coupling between the amygdala and the rostral anterior cingulate cortex (rACC2) was observed (FIG. 7A), an encouraging outcome since higher amygdala-rACC connectivity was associated with higher anxiety. This pilot finding suggested that further study of functional connectivity in the amygdala and ACC may provide insights into mechanisms-of-action of AST-120 and the metabolites it binds.

### Exploratory Efficacy Outcomes Show Improvement of Core ASD Behavior.

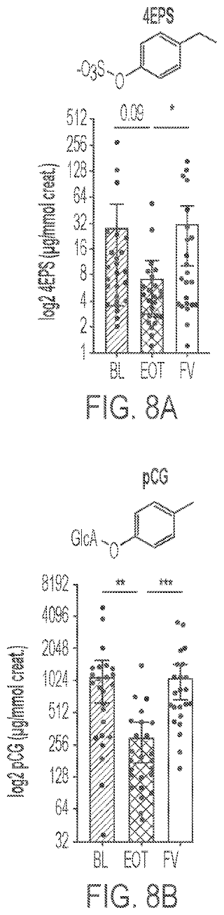

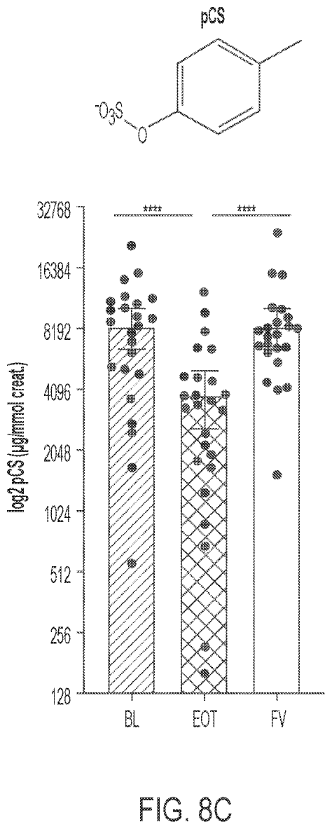

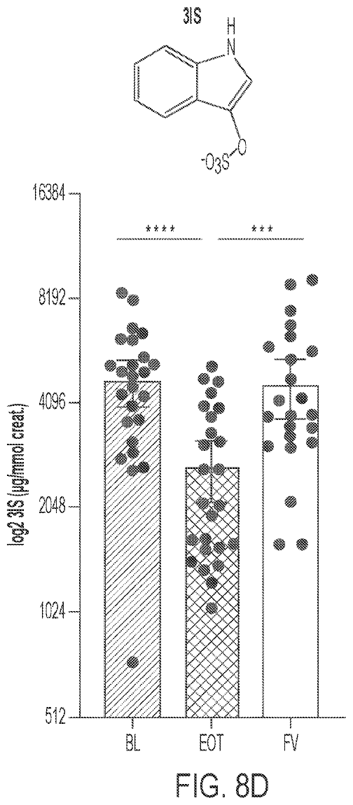

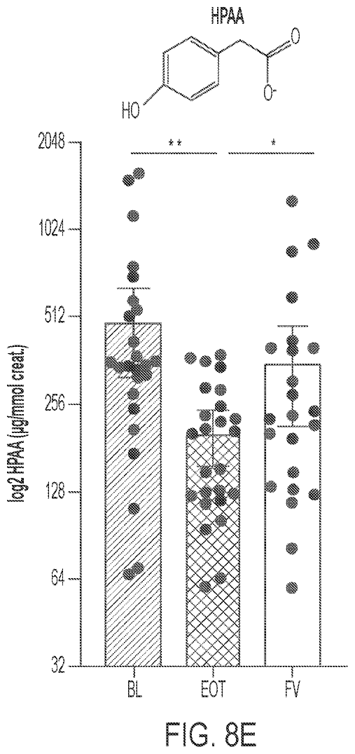

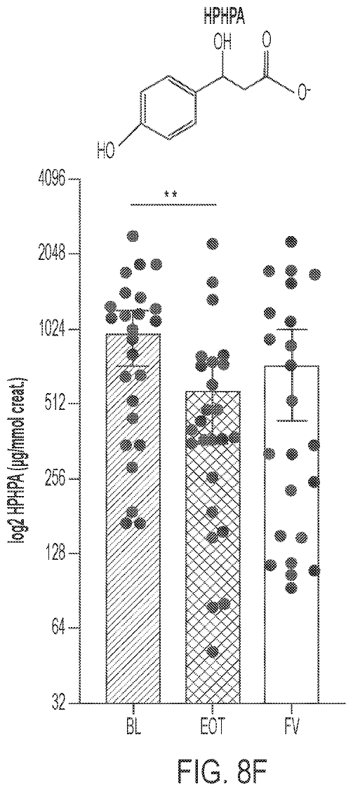

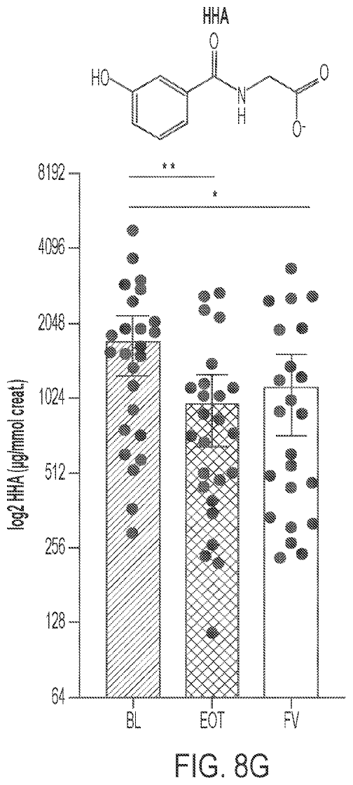

Several domains of behavioral data for all study participants were captured as exploratory endpoints. The VABS5 was administered at BL, EOT, and FV, and overall scores as well as communication and socialization scores were significantly increased by EOT (FIGS. 7B-7E). Although ten participants were removed from this analysis due to incomplete data, thereby limiting the sample size of this comparison, a mean increase of 7.8 points from BL to EOT in the Adaptive Behavior Composite score was above the minimal clinically relevant cutoff of 3.75 points (FIG. 7B). Modest indications of improved social communication and repetitive behavior of study participants was also detected in other behavioral assessments, including the SRS (FIGS. 8A-8F) and to a lesser extent in the ABC (FIGS. 8G-8J). Importantly, both assessment tools had been used to evaluate the efficacy of ASD therapeutics.

### AB-2004 Reduces Anxiety and Irritability.

The most striking behavioral outcomes of AST-120 treatment in two highly prevalent non-core domains of ASD, namely anxiety and irritability were reported. In particular, study participants with elevated BL anxiety scores ≥10, as measured by the PARS test, showed marked improvements in anxiety between initial (BL) and last (EOT) dose, a positive effect that persisted 1 month after withdrawal of drug (FV) (FIGS. 3A, 3B). These results indicated that AST-120 may be effective in treating elevated anxiety in individuals with ASD, as the minimum clinically important change in the PARS score was 15%⁷⁶. The results described herein showed an average improvement of 30% among the group with anxiety at BL, with 9 out of 15 individuals with elevated anxiety resulting in a diagnostic score that qualified as remission (score of 10 or below).

Irritability is also frequent in the ASD population and can be assessed as part of the ABC scale. A significant overall decrease in irritability as measured by the ABC-I subscale between BL and EOT was observed (FIG. 3C). In particular, individuals with high BL irritability (scores ≥15), which represented the top quartile of severity within the ASD population as a whole, displayed a remarkable 9.1 point decrease in ABC-I (FIG. 3D). The improvements at EOT were largely mitigated after drug washout (FV) in most participants (FIG. 3D). Correlations between a single metabolite and behavioral scores were not clear (Table 4). These data revealed that almost all study participants with elevated anxiety or irritability showed significant behavioral improvements following 8 weeks of treatment with AST-120.

### Discussion

Based on data from the completed open-label trial described herein, AST-120 is safe and well-tolerated for use in an adolescent ASD population, with no serious adverse events related to the drug. This study also suggested target engagement by AST-120, as evidenced by reduced levels of intestinal metabolites in plasma and urine following 8 weeks of treatment, and a general rebound to baseline levels after 4 weeks of drug washout. Further, AST-120 decreased the number participants presenting with gastrointestinal symptoms; however, it was unclear whether intestinal issues were linked to other endpoints. Though this study was powered for safety and tolerability, surprisingly, indicators of improvements in ASD-associated behaviors, namely anxiety and irritability, were observed. Decreased anxiety persisted after drug removal, whereas improvements in irritability largely returned to baseline levels by the final visit. A contribution for metabolites bound by AST-120 that were not measured here, either of host, dietary, or microbial origin, could not be excluded. Also, this study did not resolve indirect effects of drug through potential changes in nutrition, immune status, and gastrointestinal function, for example, and further proof-of-mechanism will require additional work. However, this was the first interventional study that linked phenolic metabolites in the gut with clinical features of ASD. While the preliminary evidence for improvements in behavior within this small ASD cohort were encouraging, the absence of a control arm necessitated double-blinded placebo-controlled trials to confirm efficacy of AST-120.

There are currently no approved pharmacological therapies for the treatment of the core symptoms of ASD. Two drugs, risperidone and aripiprazole, are approved by the U.S. Food and Drug Association (FDA) for treatment of irritability in ASD individuals. Irritability behaviors are common in pediatric ASD and have major implications in child development, receptivity to behavioral therapy, and child/caregiver health-related quality of life. Both drugs are atypical antipsychotic medications and are associated with a range of side effects such as somnolence, metabolic changes, weight gain, leukopenia, and tardive dyskinesia. In a phase 3 study of aripiprazole with inclusion criteria based on high irritability levels (ABC-I≥18), the response rate, or percentage of individuals with 25% improvement in ABC-I scores and a CGI-I≤2, was 49-56% in the drug arms, with a 34.7% response rate in the placebo arm. In the present study, a post-hoc analysis showed a 75% and 82% response rate in subgroups meeting somewhat similar criteria (ABC-I 18 or 15, respectively).

A placebo-controlled randomized trial is performed to test the effects of AST-120 in an ASD cohort powered to report changes in irritability.

TABLE 1
Treatment Emergent Adverse Events. n = number of subjects with
events; % = percent of subjects with events; E = number of events.

| | Adverse Event System Organ | Safety population (n = 30) | |
|---|---|---|---|
| | Class/Preferred Term | n (%) E | Severity |
| Total | Overall study | 24 (80.0%) 68 | Mild, Moderate, Severe |
| Related | Overall study | 0 (0%) 0 | N/A |
| Possibly | Overall study | 14 (46.7%) 21 | Mild, Moderate |
| related | Gastrointestinal Disorders | | |
| | Abdominal Pain | 2 (6.7%) 2 | Mild |
| | Nausca | 2 (6.7%) 2 | Mild |
| | Diarrhea | 1 (3.3%) 2 | Mild, Moderate |
| | Abdominal Discomfort | 1 (3.3%) 1 | Mild |
| | Abdominal Tenderness | 1 (3.3%) 1 | Mild |
| | Dyspepsia | 1 (3.3%) 1 | Mild |
| | Rectal Hemorrhage | 1 (3.3%) 1 | Mild |
| | Abdominal Pain Upper | 1 (3.3%) 1 | Moderate |
| | General disorders | | |
| | Pyrexia | 2 (6.7%) 2 | Mild |
| | Nervous system disorders | | |
| | Headache | 3 (10.0%) 3 | Mild |
| | Disturbance in attention | 1 (3.3%) 1 | Mild |
| | Psychiatric disorders | | |
| | Insomnia | 1 (3.3%) 1 | Moderate |
| | Anxiety | 1 (3.3%) 1 | Moderate |
| | Respiratory, Thoracic, and | | |
| | Mediastinal disorders | | |
| | Epistaxis | 1 (3.3%) 1 | Mild |
| | Skin/Subcutaneous Tissue | | |
| | disorders | | |
| | Rash | 1 (3.3%) 1 | Moderate |
| Not | Overall study | 21 (70.0%) 47 | Mild, Moderate, Severe |
| related | | | |

TABLE 2
Exclusion Criteria

Inclusion Criteria
Clinically diagnosed, documented Autism Spectrum Disorder (DSM-V criteria) confirmed with ADOS-2
at Visit 1 or within 18 months prior to Visit 1.
Adolescents ≥12 and <18 years of age at the time of consent
Subject and/or care giver must be able to read/write in English.
Able to provide developmentally and age-appropriate written informed consent as required per local
regulations for subject and/or legal guardian.
Male subjects who are post-pubertal must be sterile (surgically or otherwise) for at least 6 months or are
using single barrier contraception during the duration of the trial and up until 1 month after the last dose
AB-2004.
OR
Female subjects that are not lactating and have a negative pregnancy test at the Screening and who are
surgically sterile for at least 6 months or who agree to use double-barrier contraception, an intrauterine
device, or an oral contraceptive over the duration of the trial and at least 4 weeks after the last dose of AB-
2004
Subjects who weigh a minimum of the 5^{th}-percentile for their age at Screening
On a stable therapeutic regimen-including medication, supplements, and probiotics-for at least 30 days
prior to Screening and while participating in the trial.
Good health, as determined by the Principal Investigator (PI) or qualified designee, on the basis of medical
history, physical examination and screening laboratory results.
Good health, as determined by the Principal Investigator (PI) or qualified designee, on the basis of medical
history, physical examination and screening laboratory results.
History of gastrointestinal symptoms (diarrhea, constipation, abdominal pain, bloating) confirmed in the e-
diary with at least 50% compliance of entry for at least 14 days during the screening period.
Exclusion Criteria
History of inflammatory bowel disease, bowel obstruction, diverticulosis, or colon polyps.
Oral. injected, inhaled antibiotic within 30 days prior to Screening
Currently taking a controlled- or extended-release medication
A febrile gastrointestinal illness involving >1 day of diarrhea within 30 days prior to Screening
History of significant gastric or intestinal surgery (excluding appendectomy).
History of diabetes.
Have any clinically significant condition, is inmunocompromised, or has an unstable intercurrent illness
that would, in the opinion of the Investigator, preclude study participation or interfere with safety and
tolerability assessments of the subject.
Have an active malignancy or history of malignancy within 2 years prior to Screening.
History of significant head injury within 6 months prior to Screening.
Clinically significant blood pressure abnormalities.
Have a comorbid major psychiatric condition (e.g., schizophrenia or bipolar disorder) at Screening that in
the opinion of the Investigator may interfere with the subject's ability to complete study
procedures/comply with study requirements.
Have met criteria for a moderate to severe substance use disorder as described within the Diagnostic and
Statistical Manual of Mental Disorders V (DSM-5) criteria within 5 years prior to Screening.
Have a history of positive hepatitis B (surface antigen) and/or hepatitis C virus antibody lab results or
acquired immunodeficiency syndrome (AIDS).
Have a positive urine toxicology screen for drugs of abuse (non-prescribed amphetamines,
benzodiazepines, barbiturates, cannabinoids, or cocaine).
Have a positive alcohol test
In the investigator's opinion at significant risk for suicidal behavior
Have received any investigational product or device or participated in another investigational study within
30 days before the Screening visit; or received an investigational live bacterial product or fecal matter
transplant within 90 days before the Screening visit; or is scheduled to receive an investigational device or
another investigational drug during the course of this study.
Are from vulnerable populations, including but not limited to employees (temporary, part-time, full-time,
etc), or a family member of the research staff conducting the study, or of the Sponsor, or the Clinical
Research Organization or of the Institutional Review Board.

TABLE 3
Schedule of Assessments

| | | Screening | Treatment Period | | | | | | | Recovery |
|---|---|---|---|---|---|---|---|---|---|---|
| | | Period | Dose | Telephone | Dose | Telephone | Dose | Telephone | | Period |
| | | Screening | Period 1 | Safety | Period 2 | Safety | Period 3 | Safety | | Follow- |
| | | Visit [symbol] | Start | Check | Start | Click | Start | Check | EOT | up |
| | | Visit | | | | | | | | |
| | | V1 | | | | | | | V5 | V6 |
| | | (BL) | V2 | | V3 | | V4 | | (EOT) | (FV) |
| | | AB-2004 Trial Day | | | | | | | | |
| | | | 1 | | 15 (+5) | | 29 (+5) | | 57 (+5) | 85 (±5) |
| PROCEDURES | | −28 to −1 | Day 1 | 8 (+2) | Week 2 | 22 (+2) | Week 4 | 43 (+2) | Week 8 | Week 12 |
| Informed | | X | | | | | | | | |
| Consent/ | | | | | | | | | | |
| Assent | | | | | | | | | | |
| Demographic | | X | | | | | | | | |
| Data | | | | | | | | | | |
| Vital Signs [symbol] | | X | X | | X | | X | | X | X |
| Medical/ | | X | | | | | | | | |
| Surgical, GI | | | | | | | | | | |
| History | | | | | | | | | | |
| (including | | | | | | | | | | |
| diet), 6 [symbol] | | | | | | | | | | |
| Prior & | | X | X | X | X | X | X | X | X | X |
| Concomitant | | | | | | | | | | |
| Meds | | | | | | | | | | |
| Physical | Full | X | | | | | | | X | X |
| Exam [symbol] | Abbreviated | | X | | X | | X | | | |
| Inclusion/ | | X | X | | X | | X | | | |
| Exclusion | | | | | | | | | | |
| Criteria | | | | | | | | | | |
| Adverse | | X | X | X | X | X | X | X | X | X |
| Events | | | | | | | | | | |
| Set up | Survey | X | | | | | | | | |
| download & | training | | | | | | | | | |
| Train on | Drug diary | | X | | | | | | | |
| e-dairy [symbol] | training | | | | | | | | | |
| Review of [symbol] | | X | X | X | X | X | X | X | X | X |
| diary/paper | | | | | | | | | | |
| diary | | | | | | | | | | |
| compliance | | | | | | | | | | |
| Home | Volume & | X | X | | ◯ | | X | | X | X |
| Collection- | pH | | | | | | | | | |
| Urine and | Urine | X | X | | ◯ | | X | | X | X |
| Stool [symbol] | Biomarkers [symbol] | | | | | | | | | |
| | Stool | X | X | | ◯ | | X | | X | X |
| | Biomarkers [symbol] | | | | | | | | | |
| Blood Labs^{f} | Hematology & | X | ◯ | | ◯ | | X | | X | ◯ |
| | Serum | | | | | | | | | |
| | Chemistry | | | | | | | | | |
| | Blood | X | ◯ | | ◯ | | X | | X | ◯ |
| | Biomarkers [symbol] | | | | | | | | | |
| | Genetic | | ◯ | | | | | | | |
| | Sample | | | | | | | | | |
| Urine Labs | Urinalysis | X | ◯ | | ◯ | | X | | X | ◯ |
| | Drug & | X | ◯ | | ◯ | | X | | X | ◯ |
| | Alcohol [symbol] | | | | | | | | | |
| Pregnancy | | X | X | | ◯ | | X | | X | ◯ |
| Test [symbol] | | | | | | | | | | |
| Lactulose/ | | X | | | | | | | X | |
| Mannitol Test | | | | | | | | | | |
| Clinician | ADOS-2 (if | X | | | | | | | | |
| Questionnaires | required) | | | | | | | | | |
| | CGI-S | X | X | | | | X | | X | X |
| | CGI-I | | | | | | X | | X | X |
| | PARS | X | | | | | | | X | X |
| | Vinelands 3 | X | | | | | | | X | X |
| Patient | GSRS Dietary | X | X | | X | | X | | X | X |
| Questionnaires | Questionnaires | | | | | | | | | |
| | ABC, RBS-R, | X | | | | | | | X | X |
| | SRS-2, CASI-5 | | | | | | | | | |
| | BSS & NRS | X | X | X | X | X | X | X | X | X |
| | (daily e-diary) [symbol] | | | | | | | | | |
| Dispense/ | | | X | | X | | X | | X | |
| Collect Study | | | | | | | | | | |
| Medication | | | | | | | | | | |
| MRI Scan [symbol] | | | ◯ | | | | | | ◯ | |

S = Clinical Global Impression-Severity; EOT = End of Treatment; GI = gastrointestinal; GSRS = Gastrointestinal Symptom Rating Scale; IP = Investigational Product; MRI = magnetic resonance imaging; NRS = Numeric Rating Scale; PARS = Pediatric Anxiety Rating Scale; p-CS = p-Cresol sulfate; RBS-R = Repetitve Behaviors Scale - Revised; SRS-2 = Social Respnsiveness Scale-2; V = Visit. [symbol]
indicates data missing or illegible when filed

Key: X: Mandatory, O: Optional. Footnotes for Schedule of Assessments:
a) Vital signs included body weight, pulse rate (beats per minute), blood pressure (systolic and diastolic blood pressure, mm Hg), and respiratory rate (respirations per minute) after at least 2 minutes at rest in a supine position. Height was only to be recorded at Visit 1.
b) Physical Examination (PE)—full PE including at least general appearance, head (eyes, ears, nose, mouth, throat), skin, neurological, musculoskeletal, cardiovascular, respiratory, abdomen and extremities; abbreviated PE was symptom-directed.
c) An e-diary was used to assess GI symptoms, including pain, additional electronic patient reported outcomes (BSS and NRS), and IP dosing. A paper diary was provided to subjects to use if there were technical issues with the-diary application or if subjects were unable to complete the details in the e-diary for any reason.
d) Home collection samples. First sample was to be collected within 1 week of Visit 1 and subsequent samples were to be collected 2 to 3 days before the next scheduled visit.
e) Biomarker assessments performed in samples from blood (plasma or serum), urine, and feces. Blood: intestinal permeability (e.g., zonulin, fatty acid-binding protein 2, α-1 antitrypsin, lipopolysaccharide binding protein) and systemic inflammation (e.g., TNF-α, IL-2, IL-2R, IL-2Ra, IFN-γ, IL-4, IL-5, IL-12, IL-10, IL-13, IL-17, IL-10, IL-6, and/or IL-8). Urine: microbial and host metabolites (e.g., 4-EPS, p-CS, 5HIAA). Feces: intestinal inflammation (fecal calprotectin) and metagenomics.
f) Laboratory tests to establish eligibility were to be completed within 28 days prior to enrollment and results reviewed by the Investigator or authorized delegate before enrollment.
g) Drug of Abuse Screen—dipstick urine drug tests were screened for non-prescribed cannabis, cocaine, amphetamines, benzodiazepines, and barbiturates to ensure subjects were not self-medicating during the study. A separate urine alcohol test was performed by the central laboratory.
h) Pregnancy testing was applicable to female subjects only. The test was to be conducted on either a urine or serum sample.
i) BSS and NRS were captured in the e-diary/paper diary on a daily basis.
j) MRIs were performed on a subset of subjects (˜10). All subjects were to be offered an MRI at sites where MRIs were available.
k) Screening visit was to be conducted over 2 to 3 visits. Consent/assent, assessments to confirm eligibility, and initiation of the e-diary/paper diary were to be completed at the first part of Visit 1, whereas other tests were to be performed over a period of up to 28 days.

Abbreviations: 4-EPS=4-Ethylphenyl Sulfate; 5HIAA=5-Hydroxyindoleacetic acid; 6-GSI=Gastrointestinal Severity Index; ABC=Aberrant Behavior Checklist; ADOS-2=Autism Diagnostic Observational Schedule, Second Edition; BSS=Bristol Stool Scale; CASI-5=Child and Adolescent Symptom Inventory 5; CGI-I=Clinical Global Impression-Improvement; CGI-S=Clinical Global Impression-Severity; EOT=End of Treatment; GI=gastrointestinal; GSRS=Gastrointestinal Symptom Rating Scale; IP=Investigational Product; MRI=magnetic resonance imaging; NRS=Numeric Rating Scale; PARS=Pediatric Anxiety Rating Scale; p-CS=p-Cresol sulfate; RBS-R=Repetitive Behaviors Scale—Revised; SRS-2=Social Responsiveness Scale-2; V=Visit.

TABLE 4
Metabolite and Behavior Correlations

| | | High BL PARS | | All subjects PARS | | High BL ABC-I | | All subjects ABC-I | |
|---|---|---|---|---|---|---|---|---|---|
| | | (n = 15) | | (n = 26) | | (n = 11) | | (n = 26) | |
| Metabolite | Sample | r | p-Value | r | p-Value | r | p-Value | r | p-Value |
| 3-IS | Plasma | −0.11 | 0.70 | 0.20 | 0.35 | 0.03 | 0.89 | 0.03 | 0.89 |
| 4-EPS | Plasma | 0.35 | 0.22 | −0.16 | 0.45 | −0.03 | 0.90 | −0.03 | 0.90 |
| HHA | Plasma | 0.39 | 0.17 | −0.18 | 0.42 | 0.07 | 0.75 | 0.07 | 0.75 |
| HPAA | Plasma | −0.47 | 0.09 | −0.52 | 0.01 | −0.32 | 0.12 | −0.32 | 0.12 |
| HPHPA | Plasma | 0.37 | 0.20 | −0.27 | 0.22 | 0.04 | 0.86 | 0.04 | 0.86 |
| HPPA | Plasma | 0.42 | 0.13 | 0.29 | 0.18 | 0.02 | 0.92 | 0.02 | 0.92 |
| p-CG | Plasma | 0.08 | 0.79 | 0.18 | 0.41 | 0.10 | 0.64 | 0.10 | 0.64 |
| p-CS | Plasma | 0.00 | 0.99 | 0.31 | 0.15 | 0.05 | 0.79 | 0.05 | 0.79 |
| 3-IS | Urine | 0.32 | 0.24 | −0.01 | 0.98 | 0.02 | 0.93 | 0.02 | 0.93 |
| 4-EPS | Urine | 0.28 | 0.31 | −0.34 | 0.12 | −0.18 | 0.39 | −0.18 | 0.39 |
| HHA | Urine | 0.00 | 0.99 | −0.23 | 0.28 | −0.23 | 0.27 | −0.23 | 0.27 |
| HPAA | Urine | −0.37 | 0.17 | −0.39 | 0.06 | −0.36 | 0.07 | −0.36 | 0.07 |
| HPHPA | Urine | −0.04 | 0.88 | −0.25 | 0.26 | −0.25 | 0.23 | −0.25 | 0.23 |
| HPPA | Urine | −0.18 | 0.51 | 0.00 | 0.98 | −0.31 | 0.13 | −0.31 | 0.13 |
| p-CG | Urine | 0.13 | 0.63 | 0.22 | 0.31 | −0.05 | 0.82 | −0.05 | 0.82 |
| p-CS | Urine | 0.21 | 0.45 | 0.09 | 0.67 | −0.17 | 0.42 | −0.17 | 0.42 |

TABLE 5
Demographics (completers)

| Subject | | | | | | | ADOS |
|---|---|---|---|---|---|---|---|
| ID | Age | Wt (kg) | Wt (lb) | Ht (cm) | Ht (in) | BMI | Severity |
| 001-001 | 12.1 | 64.8 | 143 | 152 | 60 | 28.05 | 8 |
| 001-003 | 14.6 | 49.5 | 109 | 175.5 | 70 | 16.07 | 9 |
| 001-005 | 15.5 | 62.4 | 137 | 174 | 69 | 20.61 | 7 |
| 001-007 | 12.1 | 30.1 | 66 | 147 | 58 | 13.93 | 8 |
| 002-001 | 13 | 70.3 | 155 | 169 | 67 | 24.61 | 10 |
| 002-002 | 17.3 | 65.1 | 143 | 177.6 | 70 | 20.64 | Autism |
| 002-004 | 12.9 | 58.2 | 128 | 152.2 | 60 | 25.12 | 6 |
| 002-005 | 13.5 | 84 | 185 | 164.5 | 65 | 31.04 | 10 |
| 002-006 | 13.1 | 42.5 | 94 | 171.7 | 68 | 14.42 | 6 |
| 002-010 | 13.4 | 57.9 | 127 | 169.2 | 67 | 20.22 | 3 |
| 002-011 | 13.3 | 44.3 | 97 | 159 | 63 | 17.52 | 8 |
| 002-012 | 14.2 | 56.2 | 124 | 160 | 63 | 21.95 | 9 |
| 002-014 | 13.8 | 70.2 | 154 | 163 | 65 | 26.42 | 9 |
| 002-015 | 12.8 | 39.2 | 86 | 145.7 | 58 | 18.47 | 9 |
| 003-001 | 13.7 | 92.25 | 203 | 171.1 | 68 | 31.51 | 10 |
| 003-002 | 16.1 | 56.25 | 124 | 172.4 | 68 | 18.93 | 8 |
| 003-005 | 13.5 | 49 | 108 | 170.1 | 68 | 16.94 | 6 |
| 003-006 | 16.8 | 70.35 | 155 | 170.2 | 68 | 24.29 | Autism |
| 003-007 | 13.4 | 42.65 | 94 | 158.6 | 63 | 16.96 | 9 |
| 003-010 | 15 | 67.1 | 148 | 176.02 | 70 | 21.66 | 7 |
| 003-012 | 14.5 | 58.35 | 128 | 178 | 71 | 18.42 | 7 |
| 003-013 | 15.9 | 55.15 | 121 | 177.2 | 70 | 17.56 | 5 |
| 003-014 | 16.3 | 67.75 | 149 | 176.9 | 70 | 21.65 | Autism |
| 003-015 | 15.2 | 49.6 | 109 | 175.9 | 70 | 16.03 | 6 |
| 003-016 | 13.5 | 44 | 97 | 170.1 | 68 | 15.21 | 6 |
| 003-017 | 16.2 | 53.15 | 117 | 174.7 | 69 | 17.41 | Autism |
| | | | | | | | Spectrum |

Note:
ADOS severity of either Autism or Autism Spectrum is reflective of the fact that the subject was tested using Module 4 of the ADOS and as such a numerical score isn't available

TABLE 6
ABC Subscales

| | | | Subscale | Subscale III | Subscale IV | Subscale V |
|---|---|---|---|---|---|---|
| Subject | | Subscale I | II (Social) | (Stereotypic | (Hyperactivity/ | (Inappropriate |
| Number | Visit | (Irritability) | Withdrawal) | Behaviour) | Noncompliance) | Speech) |
| 001-001 | V1 (Screening) | 22 | 12 | | | |
| 001-001 | V5 (EOT/ET) | 13 | 3 | 6 | 6 | 0 |
| 001-001 | V6 | 12 | 0 | 5 | 6 | 1 |
| 001-003 | V1 (Screening) | 16 | 44 | 18 | 13 | 5 |
| 001-003 | V5 (EOT/ET) | 6 | 26 | 14 | 7 | 4 |
| 001-003 | V6 | 2 | 13 | 4 | 4 | 1 |
| 001-005 | V1 (Screening) | 3 | 9 | 3 | 4 | 6 |
| 001-005 | V5 (EOT/ET) | 5 | 9 | 1 | 4 | 4 |
| 001-005 | V6 | 2 | 6 | 1 | 5 | 6 |
| 001-007 | V1 (Screening) | 26 | 15 | 5 | 24 | 1 |
| 001-007 | V5 (EOT/ET) | 8 | 8 | 0 | 11 | 0 |
| 001-007 | V6 | 12 | 7 | 0 | 18 | 2 |
| 002-001 | V1 (Screening) | 0 | 0 | 0 | 0 | 0 |
| 002-001 | V5 (EOT/ET) | 0 | 4 | 1 | 9 | 2 |
| 002-001 | V6 | 0 | 9 | 0 | 10 | 5 |
| 002-002 | V1 (Screening) | 6 | 11 | 1 | 20 | 2 |
| 002-002 | V5 (EOT/ET) | 8 | 11 | 1 | 15 | 3 |
| 002-002 | V6 | 2 | 8 | 3 | 15 | 2 |
| 002-004 | V1 (Screening) | 24 | 22 | 5 | 22 | 4 |
| 002-004 | V5 (EOT/ET) | 15 | 4 | 4 | 14 | 1 |
| 002-004 | V6 | 16 | 7 | 2 | 17 | 4 |
| 002-005 | V1 (Screening) | 29 | 27 | 6 | 19 | 6 |
| 002-005 | V5 (EOT/ET) | 28 | 33 | 1 | 22 | 8 |
| 002-005 | V6 | 26 | 30 | 4 | 24 | 7 |
| 002-006 | V1 (Screening) | 2 | 21 | 0 | 4 | 1 |
| 002-006 | V5 (EOT/ET) | 1 | 16 | 1 | 5 | 0 |
| 002-006 | V6 | 2 | 15 | 2 | 4 | 0 |
| 002-010 | V1 (Screening) | 20 | 13 | 5 | 23 | 7 |
| 002-010 | V5 (EOT/ET) | 15 | 12 | 7 | 17 | 1 |
| 002-010 | V6 | 22 | 21 | 5 | 22 | 0 |
| 002-011 | V1 (Screening) | 1 | 0 | 0 | 2 | 0 |
| 002-011 | V5 (EOT/ET) | 1 | 0 | 0 | 2 | 0 |
| 002-011 | V6 | 1 | 0 | 0 | 0 | 0 |
| 002-012 | V1 (Screening) | 15 | 14 | 2 | 17 | 2 |
| 002-012 | V5 (EOT/ET) | 3 | 8 | 0 | 4 | 1 |
| 002-012 | V6 | 15 | 16 | 2 | 17 | 4 |
| 002-014 | V1 (Screening) | 8 | 10 | 4 | 14 | 1 |
| 002-014 | V5 (EOT/ET) | 2 | 10 | 0 | 9 | 2 |
| 002-014 | V6 | 7 | 5 | 1 | 10 | 2 |
| 002-015 | V1 (Screening) | 15 | 8 | 4 | 16 | 1 |
| 002-015 | V5 (EOT/ET) | 11 | 5 | 1 | 15 | 1 |
| 002-015 | V6 | 15 | 9 | 0 | 15 | 1 |
| 003-001 | V1 (Screening) | 40 | 27 | 6 | 36 | 7 |
| 003-001 | V5 (EOT/ET) | 17 | 8 | 0 | 12 | 3 |
| 003-001 | V6 | 37 | 19 | 3 | 27 | 3 |
| 003-002 | V1 (Screening) | 2 | 20 | 3 | 8 | 2 |
| 003-002 | V5 (EOT/ET) | 0 | 9 | 0 | 4 | 1 |
| 003-002 | V6 | 0 | 6 | 2 | 5 | 2 |
| 003-005 | V1 (Screening) | 5 | 8 | 4 | 8 | 1 |
| 003-005 | V5 (EOT/ET) | 3 | 10 | 0 | 11 | 2 |
| 003-005 | V6 | 9 | 12 | 3 | 13 | 3 |
| 003-006 | V1 (Screening) | 0 | 8 | 0 | 10 | 0 |
| 003-006 | V5 (EOT/ET) | 0 | 6 | 0 | 6 | 1 |
| 003-006 | V6 | 0 | 9 | 0 | 8 | 1 |
| 003-007 | V1 (Screening) | 24 | 20 | 10 | 26 | 8 |
| 003-007 | V5 (EOT/ET) | 24 | 21 | 8 | 25 | 7 |
| 003-007 | V6 | 25 | 19 | 10 | 34 | 7 |
| 003-010 | V1 (Screening) | 23 | 16 | 10 | 28 | 5 |
| 003-010 | V5 (EOT/ET) | 14 | 10 | 6 | 19 | 1 |
| 003-010 | V6 | 24 | 10 | 7 | 27 | 5 |
| 003-012 | V1 (Screening) | 4 | 1 | 1 | 0 | 0 |
| 003-012 | V5 (EOT/ET) | 3 | 1 | 0 | 2 | 1 |
| 003-012 | V6 | 0 | 1 | 0 | 0 | 0 |
| 003-013 | V1 (Screening) | 5 | 1 | 0 | 7 | 0 |
| 003-013 | V5 (EOT/ET) | 7 | 2 | 1 | 11 | 0 |
| 003-013 | V6 | 14 | 10 | 2 | 15 | 0 |
| 003-014 | V1 (Screening) | 2 | 14 | 6 | 2 | 2 |
| 003-014 | V5 (EOT/ET) | 4 | 13 | 4 | 9 | 3 |
| 003-014 | V6 | 1 | 12 | 3 | 5 | 2 |
| 003-015 | V1 (Screening) | 10 | 18 | 5 | 15 | 5 |
| 003-015 | V5 (EOT/ET) | 8 | 15 | 0 | 12 | 1 |
| 003-015 | V6 | 3 | 4 | 2 | 6 | 1 |
| 003-016 | V1 (Screening) | 14 | 4 | 6 | 22 | 4 |
| 003-016 | V5 (EOT/ET) | 14 | 14 | 5 | 20 | 4 |
| 003-017 | V1 (Screening) | 1 | 19 | 10 | 1 | 1 |
| 003-017 | V5 (EOT/ET) | 3 | 27 | 11 | 4 | 4 |

TABLE 7
PARS

| Subject | V1 (Baseline) | V5 (EOT) | V6 (Recovery) |
|---|---|---|---|
| 001-001 | 4 | 10 | 6 |
| 001-003 | 15 | 4 | 1 |
| 001-005 | 8 | 11 | 6 |
| 001-007 | 11 | 6 | 6 |
| 002-001 | 9 | 0 | 2 |
| 002-002 | 13 | 7 | 0 |
| 002-004 | 8 | 15 | 5 |
| 002-005 | 11 | 9 | 16 |
| 002-006 | 11 | 10 | 6 |
| 002-010 | 7 | 12 | 20 |
| 002-011 | 7 | 0 | 5 |
| 002-012 | 12 | 0 | 0 |
| 002-014 | 3 | 1 | 7 |
| 002-015 | 16 | 10 | 15 |
| 003-001 | 19 | 6 | 13 |
| 003-002 | 10 | 0 | 2 |
| 003-005 | 10 | 15 | 7 |
| 003-006 | 11 | 8 | 8 |
| 003-007 | 18 | 15 | 18 |
| 003-010 | 18 | 16 | 18 |
| 003-012 | 12 | 11 | 0 |
| 003-013 | 2 | 10 | 10 |
| 003-014 | 4 | 15 | 1 |
| 003-015 | 14 | 11 | 11 |

| | | |
|---|---|---|
| 003-016 | 11 | Subjects were not able to |
| 003-017 | 10 | complete V5 or V6 |

TABLE 8
VABS

| | | | | | | Daily | |
|---|---|---|---|---|---|---|---|
| | | | | Standardized | Communication | Living | Socialization |
| | | | Total ABC | Composite | Standard | Standard | Standard |
| age | Subject ID | | Scores | Scores | Score | Score | Score |
| 12.1 | 001-001 | V1 (Screening) | 81 | 28 | 20 | 31 | 30 |
| 12.1 | 001-001 | V5 (EOT) | 88 | 30 | 20 | 38 | 30 |
| 12.1 | 001-001 | V6 | 97 | 33 | 20 | 41 | 36 |
| 14.6 | 001-003 | V1 (Screening) | 247 | 79 | 81 | 100 | 66 |
| 14.6 | 001-003 | V5 (EOT) | 226 | 74 | 81 | 70 | 75 |
| 14.6 | 001-003 | V6 | 214 | 71 | 84 | 77 | 53 |
| 15.5 | 001-005 | V1 (Screening) | 234 | 76 | 66 | 92 | 76 |
| 15.5 | 001-005 | V5 (EOT) | 238 | 77 | 70 | 89 | 79 |
| 15.5 | 001-005 | V6 | 265 | 85 | 84 | 92 | 89 |
| 12.1 | 001-007 | V1 (Screening) | 202 | 68 | 76 | 72 | 54 |
| 12.1 | 001-007 | V5 (EOT) | 237 | 77 | 100 | 93 | 44 |
| 12.1 | 001-007 | V6 | 287 | 93 | 102 | 104 | 81 |
| 13.1 | 002-001 | V1 (Screening) | 248 | 79 | 85 | 100 | 63 |
| 13.1 | 002-001 | V5 (EOT) | 314 | 105 | 108 | 133 | 73 |
| 13.1 | 002-001 | V6 | 334 | 114 | 108 | 132 | 94 |
| 17.3 | 002-002 | V1 (Screening) | 260 | 83 | 64 | 100 | 96 |
| 17.3 | 002-002 | V5 (EOT) | 234 | 76 | 77 | 89 | 68 |
| 17.3 | 002-002 | V6 | 271 | 87 | 79 | 96 | 96 |
| 12.9 | 002-004 | V1 (Screening) | 361 | 126 | 106 | 133 | 122 |
| 12.9 | 002-004 | V5 (EOT) | 264 | 84 | 100 | 118 | 46 |
| 12.9 | 002-004 | V6 | 257 | 82 | 83 | 116 | 58 |
| 13.5 | 002-005 | V1 (Screening) | 189 | 63 | 70 | 89 | 30 |
| 13.5 | 002-005 | V5 (EOT) | 217 | 72 | 68 | 89 | 60 |
| 13.5 | 002-005 | V6 | 222 | 73 | 68 | 93 | 61 |
| 13.1 | 002-006 | V1 (Screening) | 246 | 79 | 81 | 95 | 70 |
| 13.1 | 002-006 | V5 (EOT) | 268 | 85 | 85 | 100 | 83 |
| 13.1 | 002-006 | V6 | 251 | 80 | 78 | 98 | 75 |
| 13.4 | 002-010 | V1 (Screening) | 207 | 69 | 77 | 76 | 54 |
| 13.4 | 002-010 | V5 (EOT) | 212 | 70 | 70 | 79 | 63 |
| 13.4 | 002-010 | V6 | 221 | 73 | 92 | 79 | 50 |
| 13.8 | 002-011 | V1 (Screening) | 337 | 116 | 104 | 133 | 100 |
| 13.8 | 002-011 | V5 (EOT) | 356 | 124 | 110 | 132 | 114 |
| 13.8 | 002-011 | V6 | 347 | 121 | 113 | 122 | 112 |
| 14.2 | 002-012 | V1 (Screening) | 148 | 51 | 30 | 65 | 53 |
| 14.2 | 002-012 | V5 (EOT) | 302 | 101 | 77 | 140 | 85 |
| 14.2 | 002-012 | V6 | 185 | 62 | 46 | 85 | 54 |
| 13.8 | 002-014 | V1 (Screening) | 295 | 98 | 85 | 116 | 94 |
| 13.8 | 002-014 | V5 (EOT) | 184 | 62 | 48 | 73 | 63 |
| 13.8 | 002-014 | V6 | 221 | 73 | 68 | 76 | 77 |
| 12.8 | 002-015 | V1 (Screening) | 208 | 69 | 68 | 82 | 58 |
| 12.8 | 002-015 | V5 (EOT) | 229 | 75 | 83 | 85 | 61 |
| 12.8 | 002-015 | V6 | 232 | 75 | 83 | 93 | 56 |
| 13.7 | 003-001 | V1 (Screening) | 185 | 62 | 58 | 66 | 61 |
| 13.7 | 003-001 | V5 (EOT) | 217 | 72 | 70 | 79 | 68 |
| 13.7 | 003-001 | V6 | 189 | 63 | 52 | 77 | 60 |
| 16.1 | 003-002 | V1 (Screening) | 238 | 77 | 82 | 83 | 73 |
| 16.1 | 003-002 | V5 (EOT) | 241 | 78 | 88 | 85 | 68 |
| 16.1 | 003-002 | V6 | 246 | 79 | 82 | 88 | 76 |
| 13.5 | 003-005 | V1 (Screening) | 259 | 83 | 81 | 102 | 76 |
| 13.5 | 003-005 | V5 (EOT) | 309 | 102 | 83 | 126 | 100 |
| 13.5 | 003-005 | V6 | 283 | 91 | 81 | 104 | 98 |
| 16.8 | 003-006 | V1 (Screening) | 273 | 87 | 94 | 96 | 83 |
| 16.8 | 003-006 | V5 (EOT) | 282 | 91 | 96 | 92 | 94 |
| 16.8 | 003-006 | V6 | 291 | 95 | 103 | 94 | 94 |
| 13.4 | 003-007 | V1 (Screening) | 182 | 62 | 60 | 80 | 42 |
| 13.4 | 003-007 | V5 (EOT) | 224 | 73 | 78 | 85 | 61 |
| 13.4 | 003-007 | V6 | 217 | 72 | 75 | 82 | 60 |
| 15 | 003-010 | V1 (Screening) | 202 | 68 | 75 | 79 | 48 |
| 15 | 003-010 | V5 (EOT) | 206 | 69 | 81 | 81 | 44 |
| 15 | 003-010 | V6 | 204 | 68 | 77 | 83 | 44 |
| 14.5 | 003-012 | V1 (Screening) | 93 | 32 | 32 | 23 | 38 |
| 14.5 | 003-012 | V5 (EOT) | 168 | 57 | 38 | 59 | 71 |
| 14.5 | 003-012 | V6 | 168 | 57 | 38 | 59 | 71 |
| 15.9 | 003-013 | V1 (Screening) | 227 | 74 | 75 | 92 | 60 |
| 15.9 | 003-013 | V5 (EOT) | 231 | 75 | 79 | 88 | 64 |
| 15.9 | 003-013 | V6 | 231 | 75 | 79 | 88 | 64 |
| 16.3 | 003-014 | V1 (Screening) | 255 | 82 | 88 | 109 | 58 |
| 16.3 | 003-014 | V5 (EOT) | 257 | 82 | 91 | 96 | 70 |
| 16.3 | 003-014 | V6 | 292 | 96 | 85 | 111 | 96 |
| 15.2 | 003-015 | V1 (Screening) | 220 | 72 | 79 | 70 | 71 |
| 15.2 | 003-015 | V5 (EOT) | 263 | 84 | 96 | 92 | 75 |
| 15.2 | 003-015 | V6 | 242 | 78 | 82 | 77 | 83 |
| | 003-016 | Analysis not completed due to parental illness | | | | | |
| | 003-017 | | | | | | |

Note:
Bold subject numbers not included because their assessments were not valid based on assessment criteria

TABLE 9
SRS

| | | SRS | Social | Social | Social | Social | Repetitive |
|---|---|---|---|---|---|---|---|
| Subject | interval_name | Composite | Awareness | Cognition | Communication | Motivation | Behavior |
| 001-001 | V1 (Screening) | 80 | 89 | 81 | 69 | 62 | 89 |
| 001-001 | V5 (EOT/ET) | 74 | 73 | 70 | 67 | 62 | 87 |
| 001-001 | V6 | 68 | 73 | 70 | 63 | 58 | 68 |
| 001-003 | V1 (Screening) | 98 | 76 | 87 | 100 | 93 | 98 |
| 001-003 | V5 (EOT/ET) | 85 | 73 | 76 | 86 | 77 | 89 |
| 001-003 | V6 | 85 | 76 | 74 | 90 | 79 | 78 |
| 001-005 | V1 (Screening) | 70 | 51 | 72 | 74 | 62 | 68 |
| 001-005 | V5 (EOT/ET) | 68 | 45 | 66 | 68 | 67 | 75 |
| 001-005 | V6 | 69 | 54 | 74 | 67 | 62 | 71 |
| 001-007 | V1 (Screening) | 92 | 79 | 91 | 89 | 87 | 87 |
| 001-007 | V5 (EOT/ET) | 81 | 81 | 81 | 83 | 71 | 68 |
| 001-007 | V6 | 71 | 67 | 66 | 71 | 75 | 62 |
| 002-001 | V1 (Screening) | 58 | 60 | 52 | 56 | 62 | 61 |
| 002-001 | V5 (EOT/ET) | 71 | 67 | 65 | 69 | 71 | 71 |
| 002-001 | V6 | 70 | 67 | 61 | 75 | 58 | 71 |
| 002-002 | V1 (Screening) | 75 | 54 | 72 | 74 | 77 | 76 |
| 002-002 | V5 (EOT/ET) | 76 | 73 | 74 | 75 | 67 | 73 |
| 002-002 | V6 | 69 | 54 | 63 | 72 | 73 | 66 |
| 002-004 | V1 (Screening) | 64 | 67 | 59 | 58 | 62 | 71 |
| 002-004 | V5 (EOT/ET) | 75 | 86 | 72 | 77 | 67 | 64 |
| 002-004 | V6 | 70 | 76 | 70 | 70 | 56 | 68 |
| 002-005 | V1 (Screening) | 96 | 95 | 96 | 91 | 95 | 82 |
| 002-005 | V5 (EOT/ET) | 86 | 86 | 91 | 78 | 79 | 82 |
| 002-005 | V6 | 91 | 89 | 87 | 90 | 79 | 87 |
| 002-006 | V1 (Screening) | 75 | 79 | 66 | 74 | 73 | 71 |
| 002-006 | V5 (EOT/ET) | 72 | 54 | 66 | 69 | 77 | 76 |
| 002-006 | V6 | 73 | 64 | 61 | 73 | 75 | 76 |
| 002-010 | V1 (Screening) | 87 | 82 | 81 | 81 | 81 | 89 |
| 002-010 | V5 (EOT/ET) | 80 | 73 | 76 | 74 | 69 | 89 |
| 002-010 | V6 | 88 | 86 | 81 | 85 | 79 | 89 |
| 002-011 | V1 (Screening) | 62 | 51 | 65 | 62 | 54 | 68 |
| 002-011 | V5 (EOT/ET) | 58 | 57 | 63 | 55 | 46 | 66 |
| 002-011 | V6 | 54 | 57 | 57 | 55 | 42 | 57 |
| 002-012 | V1 (Screening) | 83 | 79 | 79 | 81 | 87 | 73 |
| 002-012 | V5 (EOT/ET) | 74 | 73 | 79 | 74 | 69 | 61 |
| 002-012 | V6 | 88 | 82 | 87 | 90 | 83 | 73 |
| 002-014 | V1 (Screening) | 73 | 64 | 79 | 73 | 62 | 69 |
| 002-014 | V5 (EOT/ET) | 85 | 73 | 83 | 93 | 69 | 76 |
| 002-014 | V6 | 74 | 76 | 78 | 70 | 62 | 73 |
| 002-015 | V1 (Screening) | 76 | 76 | 78 | 70 | 69 | 76 |
| 002-015 | V5 (EOT/ET) | 73 | 73 | 76 | 70 | 67 | 68 |
| 002-015 | V6 | 70 | 67 | 72 | 67 | 67 | 68 |
| 003-001 | V1 (Screening) | 79 | 70 | 79 | 79 | 67 | 80 |
| 003-001 | V5 (EOT/ET) | 83 | 76 | 89 | 84 | 67 | 75 |
| 003-001 | V6 | 74 | 60 | 78 | 78 | 67 | 66 |
| 003-002 | V1 (Screening) | 75 | 79 | 63 | 84 | 64 | 66 |
| 003-002 | V5 (EOT/ET) | 70 | 76 | 57 | 78 | 71 | 53 |
| 003-002 | V6 | 71 | 79 | 57 | 78 | 69 | 59 |
| 003-005 | V1 (Screening) | 77 | 73 | 72 | 79 | 75 | 69 |
| 003-005 | V5 (EOT/ET) | 76 | 73 | 72 | 81 | 75 | 59 |
| 003-005 | V6 | 78 | 76 | 76 | 81 | 73 | 64 |
| 003-006 | V1 (Screening) | 52 | 57 | 48 | 44 | 54 | 62 |
| 003-006 | V5 (EOT/ET) | 59 | 51 | 52 | 58 | 60 | 68 |
| 003-006 | V6 | 61 | 64 | 50 | 61 | 58 | 68 |
| 003-007 | V1 (Screening) | 87 | 79 | 85 | 85 | 79 | 82 |
| 003-007 | V5 (EOT/ET) | 86 | 82 | 83 | 91 | 71 | 78 |
| 003-007 | V6 | 85 | 73 | 83 | 85 | 77 | 80 |
| 003-010 | V1 (Screening) | 82 | 82 | 79 | 79 | 71 | 80 |
| 003-010 | V5 (EOT/ET) | 80 | 86 | 78 | 80 | 64 | 76 |
| 003-010 | V6 | 72 | 79 | 74 | 72 | 58 | 66 |
| 003-012 | V1 (Screening) | 69 | 64 | 59 | 67 | 71 | 73 |
| 003-012 | V5 (EOT/ET) | 67 | 60 | 65 | 70 | 67 | 61 |
| 003-012 | V6 | 58 | 57 | 59 | 52 | 60 | 59 |
| 003-013 | V1 (Screening) | 60 | 60 | 55 | 66 | 54 | 57 |
| 003-013 | V5 (EOT/ET) | 59 | 76 | 57 | 65 | 46 | 48 |
| 003-013 | V6 | 58 | 64 | 57 | 68 | 42 | 52 |
| 003-014 | V1 (Screening) | 83 | 86 | 74 | 81 | 83 | 76 |
| 003-014 | V5 (EOT/ET) | 68 | 79 | 59 | 67 | 69 | 61 |
| 003-014 | V6 | 68 | 79 | 61 | 68 | 69 | 61 |
| 003-015 | V1 (Screening) | 81 | 79 | 78 | 82 | 67 | 78 |
| 003-015 | V5 (EOT/ET) | 76 | 70 | 76 | 77 | 71 | 68 |
| 003-015 | V6 | 77 | 70 | 78 | 75 | 71 | 75 |

### Example 2

### Exploratory Analysis of Metabolite Biomarkers or Composite Biomarkers

An exploratory analysis was conducted to determine the nature of the relationship between change in metabolites from Baseline to Week 8 and change in assessment scores over that period, for subjects who participated in AXL-1224-2004-001. This was an open-label, single-arm, multiple ascending dose study.

### Objectives:

(1) To determine which biomarker changes from Baseline to Week 8 were most strongly related to change in assessment score for Aberrant Behavior Checklist—Irritability (ABC-I) and Pediatric Anxiety Rating Scale (PARS), within three overlapping subject populations.
(2) To measure evidence that changes in biomarkers are generally related to changes in assessment score.

Population analysis was conducted among subjects who met the following conditions: all subjects who completed the study through week 8 (N=26), subjects who had a screening ABC-I score of 15 (N=11) or higher or a screening PARS score of 10 or higher (N=15), and subjects who had a Screening ABC-I score of 15 or higher and a screening PARS score of 10 or higher (N=8).

The response variables included: Change in assessment score, calculated as raw change (Week 8 score—Screening score) in ABC-I score and PARS score. The covariate was the baseline score (ABC-I or PARS). The predictor variable was calculated as follows: Change in biomarker=log₂-Fold Change (log 2(Week 8 value/Baseline value)) for 12 biomarkers measured in two specimen types (Serum and Urine)—22 total combinations. The baseline value was the average of the screening and day 1 values, when both were available.

### Analysis for Objective 1

The squared partial correlation between each response variable and each predictor variable, adjusting for baseline assessment score, was calculated to measure the strength of the relationships between the change in assessment scores and the change in biomarkers, for each analysis population. The theoretical range for these values was between 0 (little to no relationship) and 1 (a perfect linear relationship). Higher values represented a stronger relationship.

The relationship between assessment and biomarker varied depending on the assessment, biomarker, and analysis population. Biomarkers 4-EPS, INDPYR, N-AS generally showed the strongest relationship with ABC-I across analysis populations. Biomarkers HPAA, N-AS, and p-CS generally showed the strongest relationship with PARS across analysis populations.

### Analysis for Objective 2

Following Objective 1, The top k biomarkers for each assessment, in terms of squared partial correlation, were selected for a multiple regression analysis. For each analysis population, k was chosen to be N/3, rounded down, to mitigate the risk overfitting. These biomarkers, along with baseline score, were used to predict the change in assessment score.

Across analysis populations and assessments, the biomarker+baseline-score models outperformed the baseline-score-only models by between 10-50 percentage points in multiple-R². This implied that changes in several of the exploratory biomarkers contain important, unique, and potentially predictive information about changes in assessment scores.

### ASD Metabolomics Assays and Sample Analysis

Samples from the AST-120 Phase 1b/2a study was analyzed. Targeted analysis was conducted in urine (11 analytes; IPA removed) and plasma (12 analytes).

A quantitative assay for 4-EPS and pCS was developed. Quantitative LC-MS assay were developed for 4-EPS and pCS for human urine and plasma to provide relatively quick and cost-effective generation of “fast turnaround” data.

Urine and plasma samples for 4-EPS and pCS were analyzed.

Targeted analysis was carried out in urine (11 analytes) and plasma (12 analytes)

### ASD Targeted Metabolite Panel History

The original panel targeted 14 metabolites that were selected based on: structural similarity to 4-EPS and putative role in or correlation with behavioral phenotypes (ASD, anxiety, executive function, etc.). The metabolites were 4-EP: 4-ethyl phenol, 4-EPS: 4-ethylphenyl sulfate, pC: para-cresol, pCS: para-cresyl sulfate, pCG: p-cresyl glucuronide, 3-IS (3-indoxyl sulfate), NAS: N-acetylserine, Indole pyruvate, IPA: Imidazole propionate, CMPF: 3-Carboxy-4-methyl-5-propyl-2-furanpropionic acid, 3-HHA: 3-hydroxyhippuric acid or 3-hydroxyhippurate, 3-HPHPA: 3-(3-hydroxyphenyl)-3-hydroxyproprionic acid, HPPA: 4-hydroxyphenylpropionic acid, and HPAA: 3-hydroxyphenylacetic acid or 3-hydroxyphenylacetate.

In the revised targeted panel, a down-selected targeted panel based on lack or prevalence in ASD samples or inability to validate technically in 11 urine and 12 plasma samples. The metabolites 4-EP and pC were excluded. Indole pyruvate was removed from both the urine and plasma panel and Imidazole propionate (IPA) was removed from the plasma panel. The metabolites that were included were 4-EPS, pCS, pCG: p-cresol glucuronide, 3-IS, NAS: N-acetylserine, CMPF: 3-Carboxy-4-methyl-5-propyl-2-furanpropionic acid, 3-HHA: 3-hydroxyhippuric acid or 3-hydroxyhippurate, 3-HPHPA: 3-(3-hydroxyphenyl)-3-hydroxyproprionic acid, HPPA: 4-hydroxyphenylpropionic acid, and HPAA: 3-hydroxyphenylacetic acid or 3-hydroxyphenylacetate.

TABLE 10
Autism metabolite panel - Urine

| | Quantitation Range | | Observed Range in | | | No. samples |
|---|---|---|---|---|---|---|
| | from Validation | | Clinical Samples after | | Urine | ALOQ or |
| Analyte | (Urine) | | 10-x dilution per method | | Method | BLOQ |
| 4-Ethylphenyl sulfate (4- | 1.00-400 | ng/mL | 0.85-423 | ng/mL | 3 | 23/151 ALOQ |
| EPS) | | | | | | |
| p-Cresol sulfate (pCS) | 50.0-20,000 | ng/mL | 903-769,000 | ng/mL | 1 | 13/151 ALOQ |
| Imidazolepropionate (IPA) | 15.0-6,000 | ng/mL | 50.4-5,640 | ng/mL | 4 | 0/151 |
| N-Acetylserine | 5.00-2,000 | ng/mL | 362-4,650 | ng/mL | 2 | 0/151 |
| 3-Carboxy-4-methyl-5- | 2.50-1,000 | ng/mL | BLOQ-2,100 | ng/mL | 3 | 1/151 ALOQ |
| propyl-2-furanpropionic | | | | | | 1/151 BLOQ |
| acid (CMPF) | | | | | | |
| 3-Hydroxyhippurate (HHA) | 20.0-8,000 | ng/mL | 112-116,600 | ng/mL | 2 | 4/151 ALOQ |
| 3-Hydroxyphenylacetate | 2.50-1,000 | ng/mL | 37.4-26,700 | ng/mL | 2 | 15/151 ALOQ |
| (HPAA) | | | | | | |
| 3-(3-Hydroxyphenyl)-3- | 20.0-8,000 | ng/mL | 54.8-69,500 | ng/mL | 2 | 2/151 ALOQ |
| hydroxypropionate | | | | | | |
| (HPHPA) | | | | | | |
| 3-Indoxyl sulfate | 50.0-20,000 | ng/mL | 555-53,700 | ng/mL | 1 | 2/151 ALOQ |
| 3-(4- | 0.500-200 | ng/mL | BLOQ-95.1 | ng/mL | 2 | 40/151 BLOQ |
| Hydroxyphenyl)prepionate | | | | | | |
| (HPPA) | | | | | | |
| p-Cresol glucuronide (pCG) | 10.0-4,000 | ng/mL | BLOQ- 170,000 | ng/mL | 1 | 6/151 ALOQ |
| | | | | | | 1/151 BLOQ |

| | | | | |
|---|---|---|---|---|
| Indolepyruvate | N/A | N/A | N/A | N/A |

Indole pyruvate was not included in final urine assay. A quantitative assay was unable to be developed either with or without derivatization. Furthermore, it was shown that the compound was unstable in the analysis solution while in the autosampler. See validation report for more context and detail. All urine samples were diluted 10−x as part of the method. Observed ranges shown were what assay result showed. The actual original concentration in urine sample was 10−x higher. Quantitation ranges (100 or 400-fold) were established based on the analysis of 6 male and 6 female plasma and urine samples. In some embodiments, quantification ranges were established by the lower limits of assay sensitivity.

TABLE 11
Autism metabolite panel - PLASMA

| | Quantitation Range | | Quantitation Range | | Urine | Plasma |
|---|---|---|---|---|---|---|
| Analyte | (Urine) | | (Plasma) | | Method | Method |
| 4-Ethylphenyl sulfate (4-EPS) | 1.00-400 | ng/mL | 1.00-400 | ng/mL | 3 | 2 |
| p-Cresol sulfate (pCS) | 50.0-20,000 | ng/mL | 25.0-10,000 | ng/mL | 1 | 2 |
| Imidazolepropionate (IPA) | 15.0-6,000 | ng/mL | 3.0-300 | mg/mL | 4 | 1 |
| N-Acetylserine | 5.00-2,000 | ng/mL | 7.50-3,000 | ng/mL | 2 | 3 |
| 3-Carboxy-4-methyl-5-propyl-2- | 2.50-1,000 | ng/mL | 5.00-2,000 | ng/mL | 3 | 2 |
| furanpropionic acid (CMPF) | | | | | | |
| 3-Hydroxyhippurate (HHA) | 20.0-8,000 | ng/mL | 2.00-800 | ng/mL | 2 | 3 |
| 3-Hydroxyphenylacetate | 2.50-1,000 | ng/mL | 2.00-200 | ng/mL | 2 | 3 |
| (HPAA) | | | | | | |
| 3-(3-Hydroxyphenyl)-3- | 20.0-8,000 | ng/mL | 0.750-300 | ng/mL | 2 | 3 |
| hydroxypropionate (HPHPA) | | | | | | |
| 3-Indoxyl sulfate | 50.0-20,000 | ng/mL | 40.0-4,000 | ng/mL | 1 | 2 |
| 3-(4-Hydroxyphenyl)propionate | 0.500-200 | ng/mL | 0.500-200 | ng/mL | 2 | 3 |
| (HPPA) | | | | | | |
| p-Cresol glucuronide (pCG) | 10.0-4,000 | ng/mL | 2.00-800 | ng/mL | 1 | 2 |

| | | | | |
|---|---|---|---|---|
| Indolepyruvate | N/A | 50.0-5,000 | ng/mL | 3 |

Indole pyruvate was omitted from the plasma panel due to observed run-to-run (lot-to-lot) performance issues observed in analysis of AXL-1224-2004-001 study samples. IPA was omitted from the plasma panel due to low hit-rate (only 2/139 samples were positive in CHARGE quantitative analysis). Quantitation ranges (100 or 400-fold) were established based on the analysis of 6 male and 6 female plasma and urine samples. In some embodiments, quantification ranges were established by the lower limits of assay sensitivity.

TABLE 12
Autism metabolite panels - validation overview

| The assay validation consisted of three analytical runs | | |
|---|---|---|
| which addressed the following performance parameters: | Urine Assays | Plasma Assays |
| Linearity, Accuracy, and Precision of Calibration Standards | Pass (all) | Pass (all) |
| Accuracy and Precision at the Lower Limit of Quantitation | Pass (all) | Pass (all) |
| A curacy and Precision of Quality Control Samples | Pass (all <10% Inter- | Pass fall <10% Inter- |
| | assay CV) | assay CV) |
| Sample Stability | | |
| Bench-top stability (3 h and 6 h at room temperature; | Pass (all <10% Bias) | Pass (all <10% Bias) |
| 3 h on ice) | | |
| Freeze-Thaw stability (3 freeze-thaw cycles) | Pass (most <3% Bias; | Pass (all <10% Bias) |
| | CMPF −11.6%) | |
| Relative Recovery | Pass (all, range 91 to | Fail for Indolepruvate |
| | 107%) | (141%), all other |
| | | passed (95-105% |
| | | recovery) |
| Carryover | All Passed, but some | All Passed, but some |
| | carryover noted for CMPF | carryover noted for |
| | (18.5%), HPAA (6.5%), | CMPF (9.4%) and |
| | HPHPA (5.1%), and | Indolepyruvate |
| | HPPA (7.6%) | (24.5%) |

### Autism Metabolite Panels—Assay Details

### Urine Methods

For Assay 1, samples were prepared by diluting human urine 10-fold [10 μL urine+90 μL PBS]. Samples were spiked with an Internal Standard [+40 ul Internal Standard Working Solution (WIS8)]. A solvent crash was performed using ACN/MeOH extraction. An aliquot was removed for Method 1 [150 μL]. An aliquot was derivatized for Method 2 using 100 μL of the ACN/MeOH extracted sample.

In method 1, LCMS was conducted by injecting aliquot onto Agilent 1290/AB Sciex 5500 QTrap LC-MS/MS with UHPLC C18 column. pCS, 3-IS, and pCG were measured in Negative Mode. In method 2, LCMS was conducted by injecting aliquot onto Agilent 1290/AB Sciex 5500 QTrap LC-MS/MS with UHPLC C18 column. N-AS, HPA, HPAA, HPHPA, and HPAA were measured in Negative mode.

For Assay 2, samples were prepared using undiluted urine [50 μL]. The samples were spiked with an Internal Standard [+20 μl Internal Standard Working Solution (WIS3)]. A solvent crash was performed using ACN/MeOH extraction. An aliquot was removed for 2 native LC/MS methods [3 and 4; 150 μL].

In method 3, LCMS was conducted by injecting aliquot onto Agilent 1290/AB Sciex 5500 QTrap LC-MS/MS with UHPLC C18 column. 4-EPS and CMPF were measured in Negative Mode. In method 4, LCMS was conducted by injecting aliquot onto Agilent 1290/AB Sciex 5500 QTrap LC-MS/MS with a second UHPLC C18 column. Imidaxolepropionate (IPA) was measured in Positive Mode.

### Plasma Methods

All 3 samples were prepared using undiluted plasma [50 μL] and spiked with Internal Standard [+20 μL WIS]. Protein was precipitated using ACN/MeOH extraction and spun. 100 μL of aliquot was removed for 2 native LC/MS methods, methods 1 and 2. An aliquot was derivatized for Method 3 using 100 μL of the ACN/MeOH extracted sample.

In method 2, LCMS was conducted by injecting aliquot onto Agilent 1290/AB Sciex 5500 QTrap LC-MS/MS with UHPLC C18 column. pCS, 3-IS, pCG, 4-EPS, and CMPF were measured in Negative Mode. In method 3, LCMS was conducted by injecting aliquot onto Agilent 1290/AB Sciex 5500 QTrap LC-MS/MS with UHPLC C18 column. N-AS, HPA, HPAA, HPHPA, and HPAA were measured in Negative mode.

### Results

### 1. Composite Biomarkers for Patient Identification (Likelihood of Responding to Treatment)

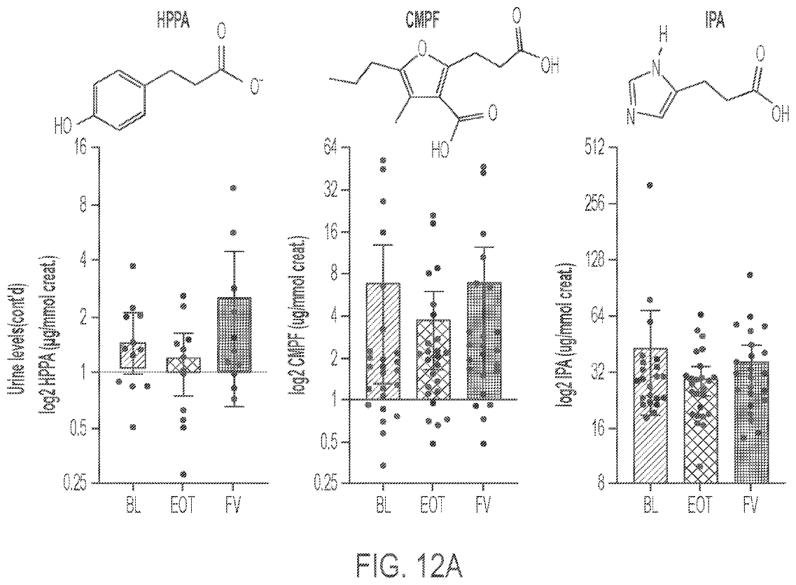

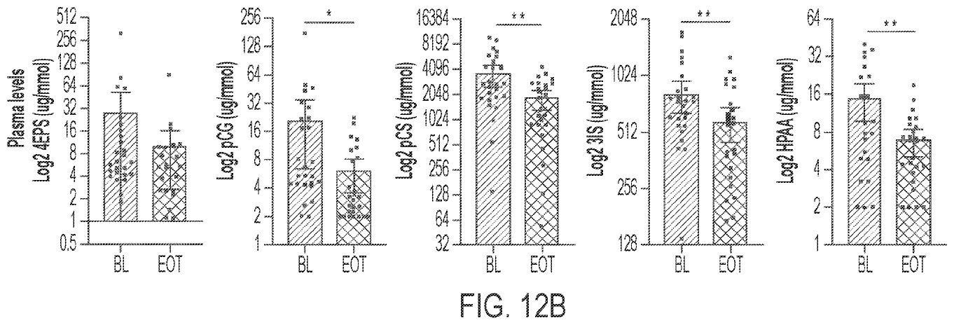

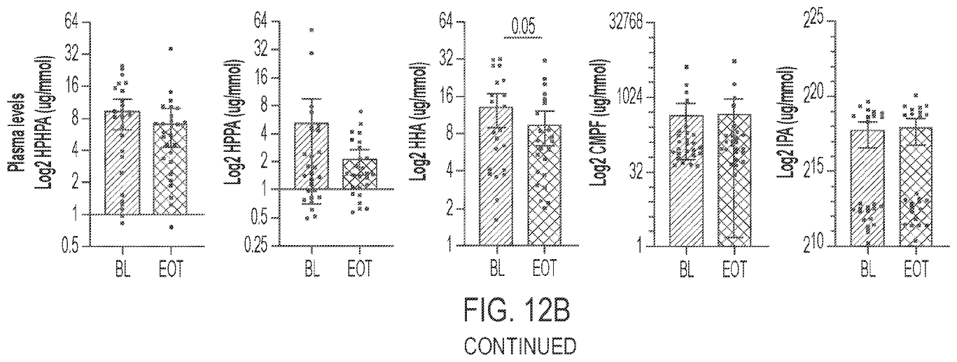

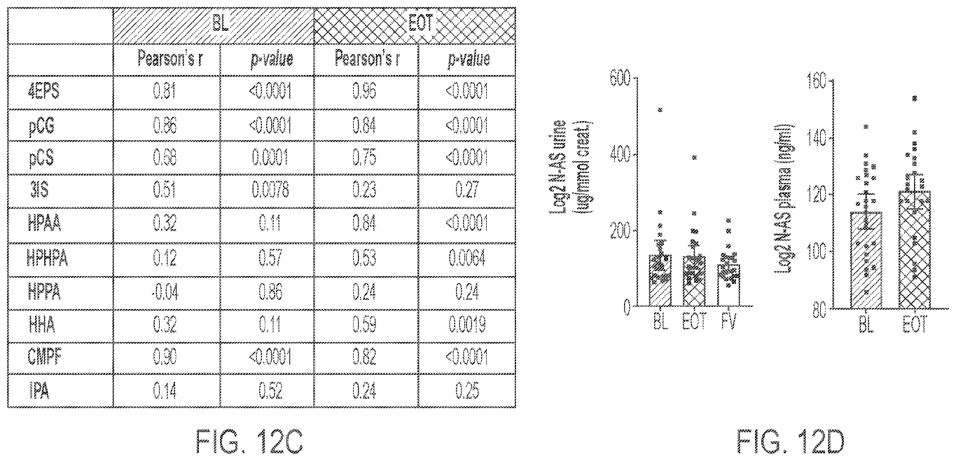

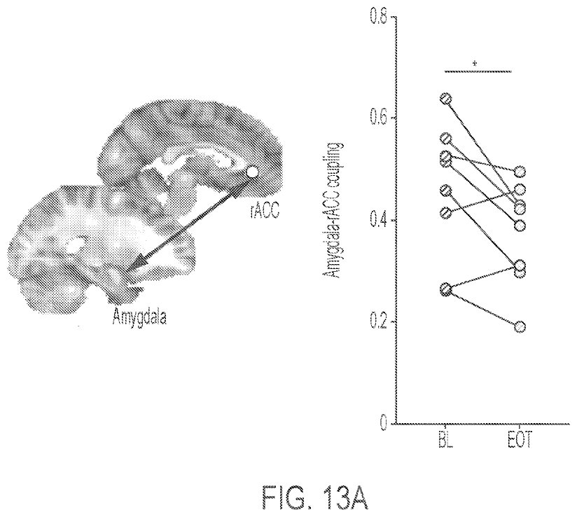

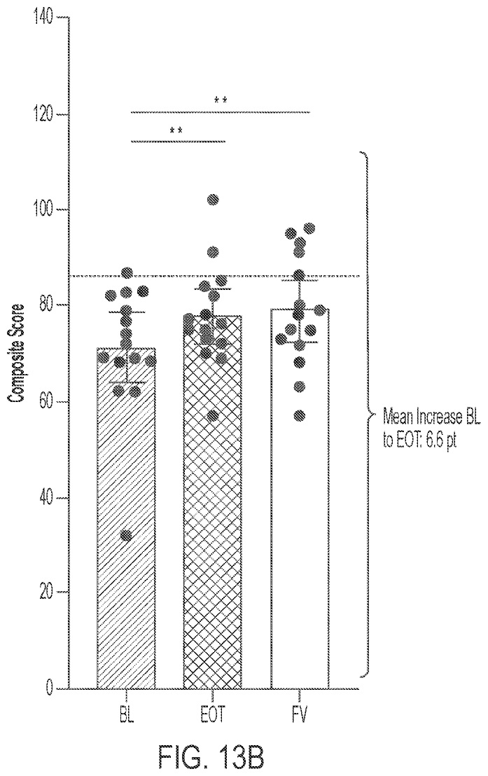

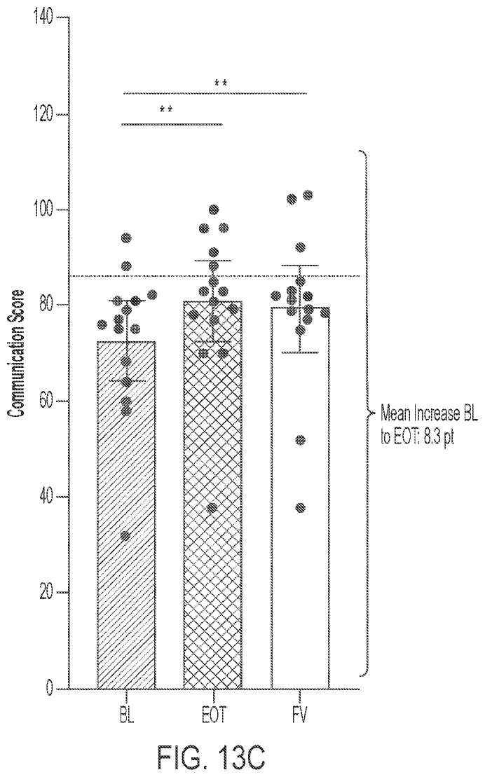

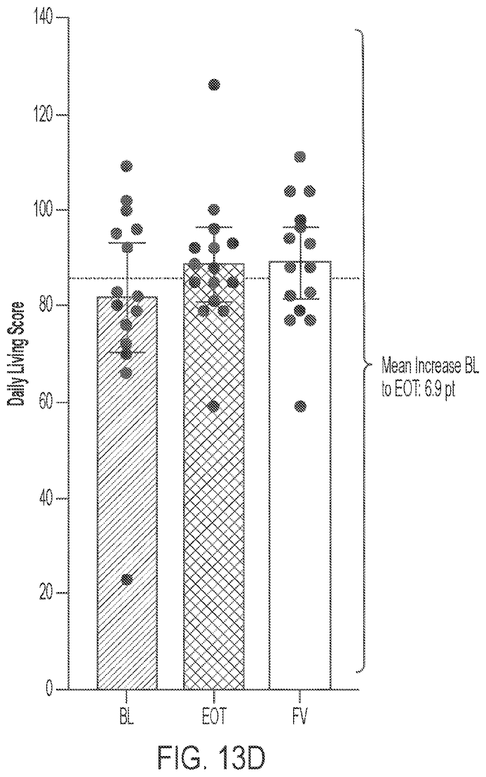

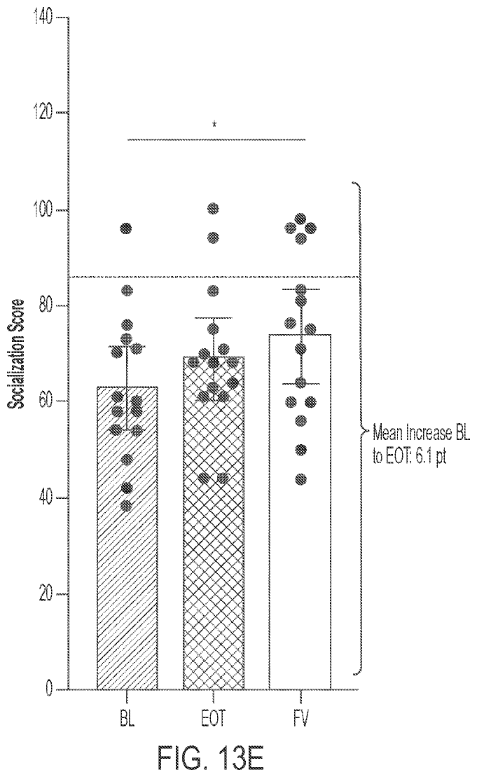

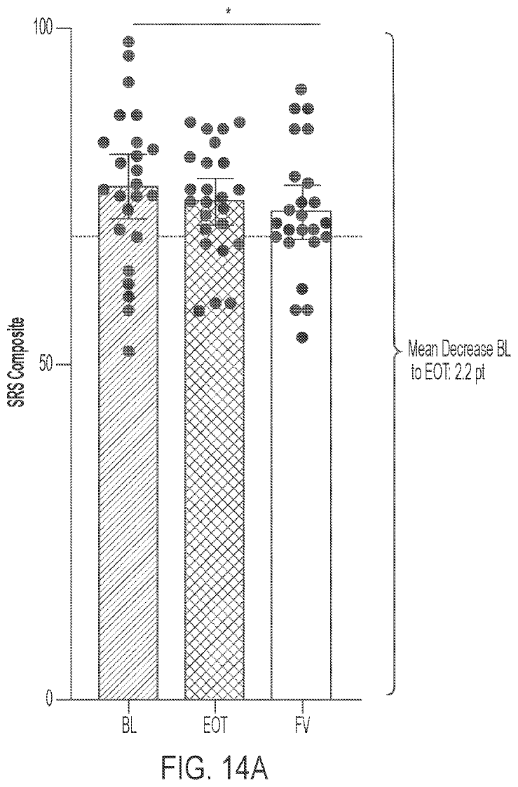

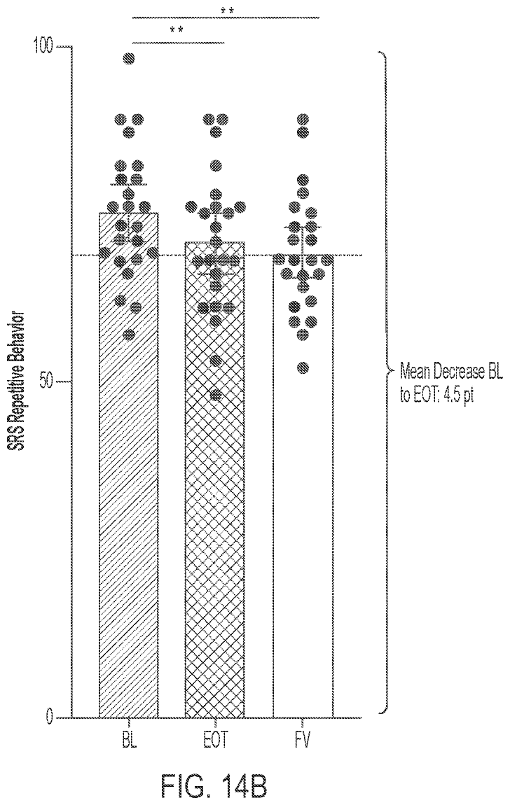

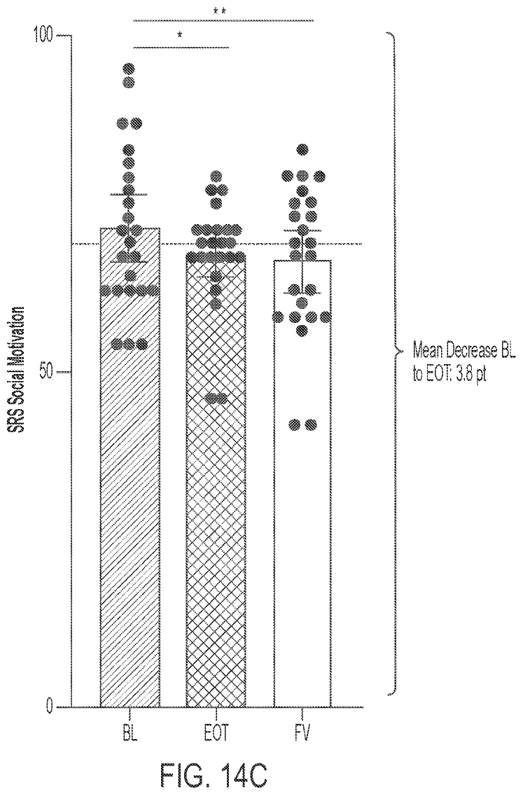

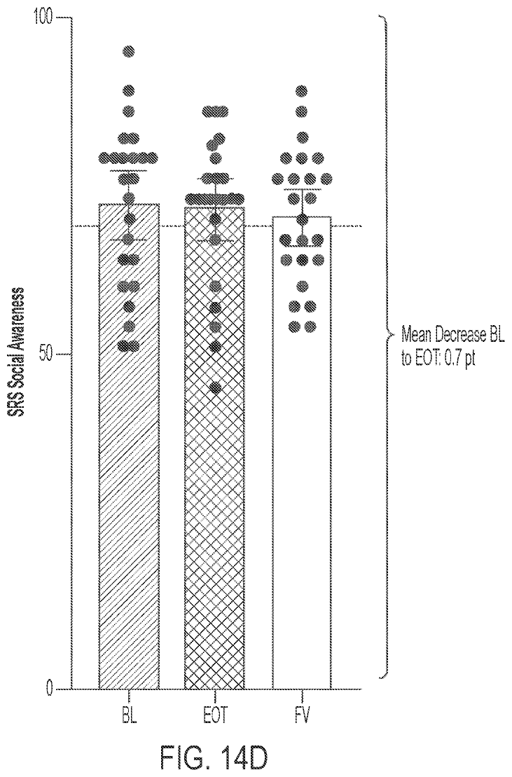

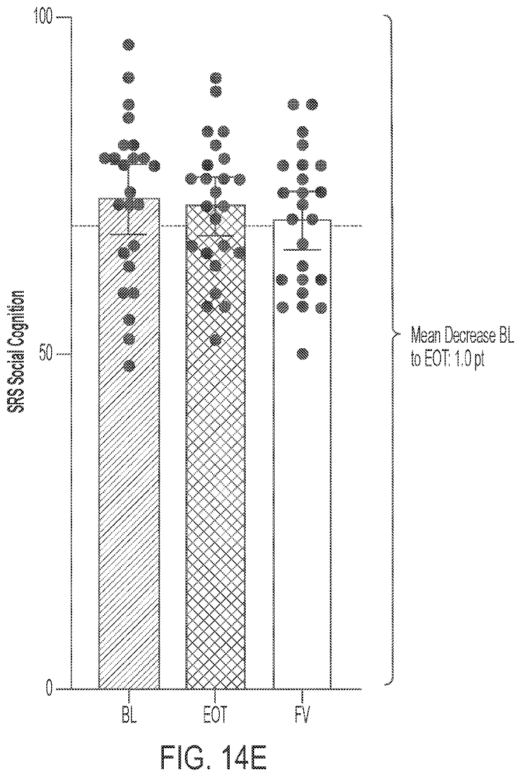

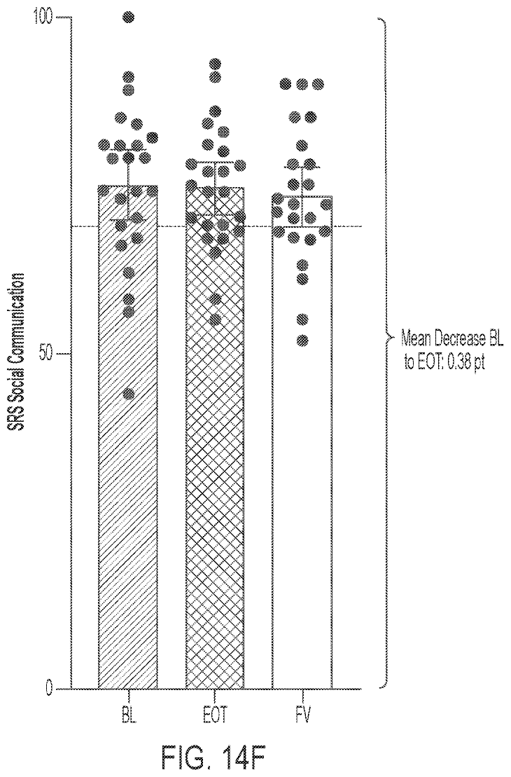

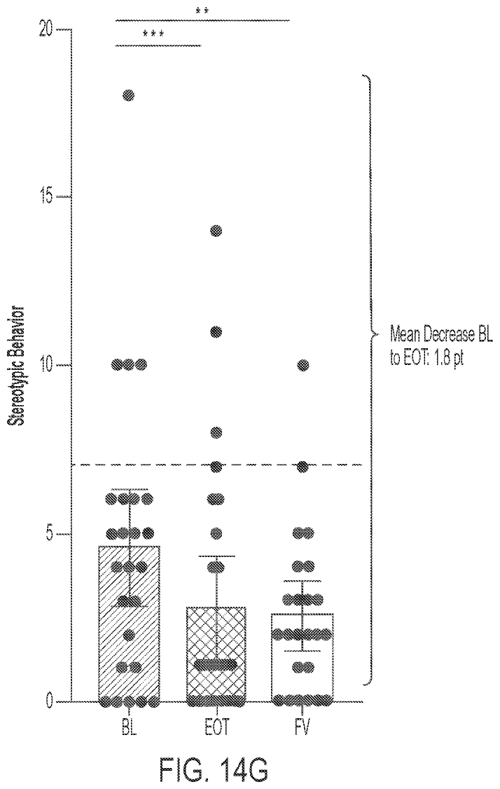

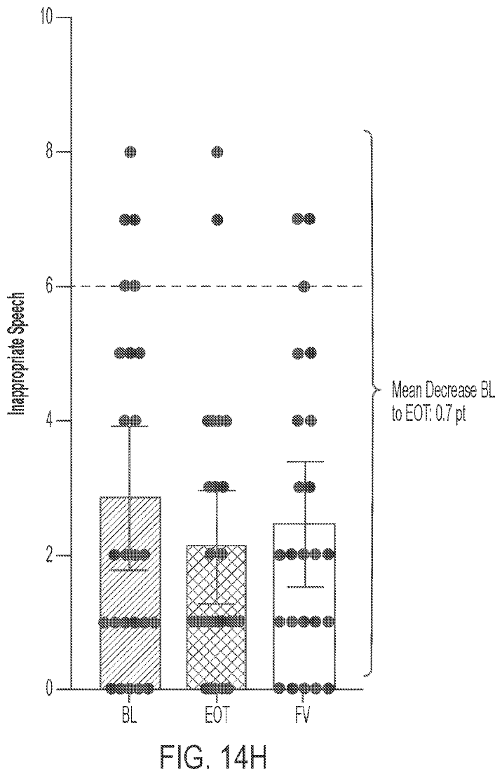

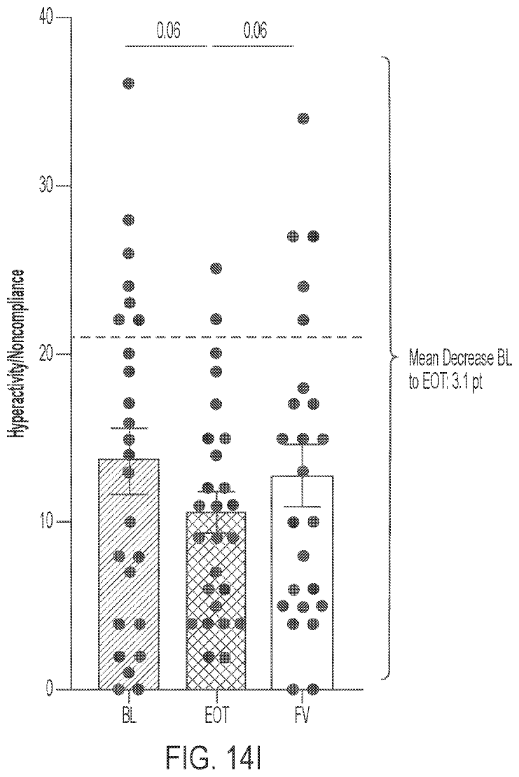

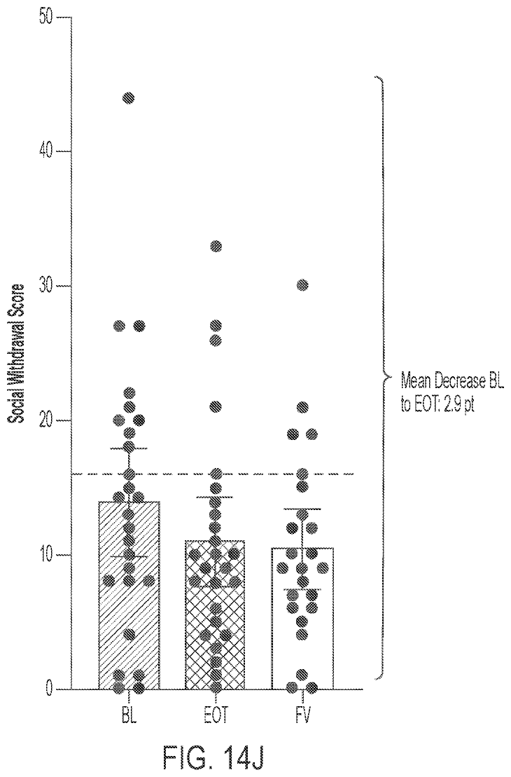

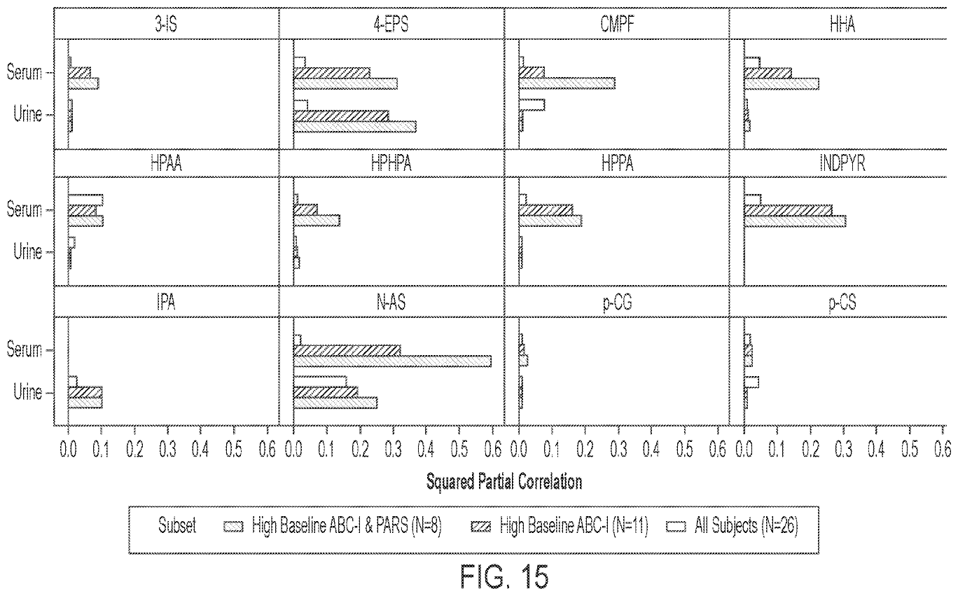

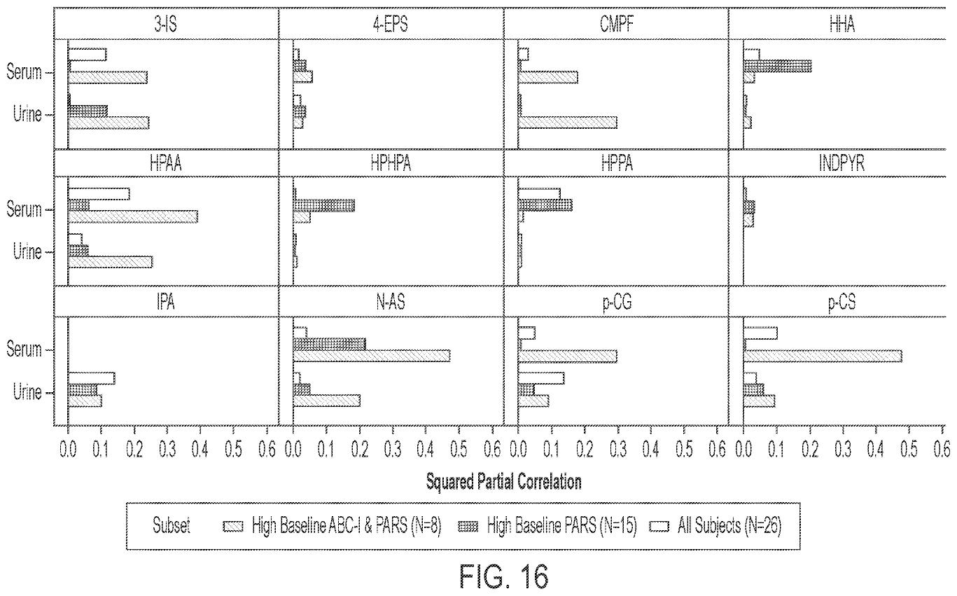

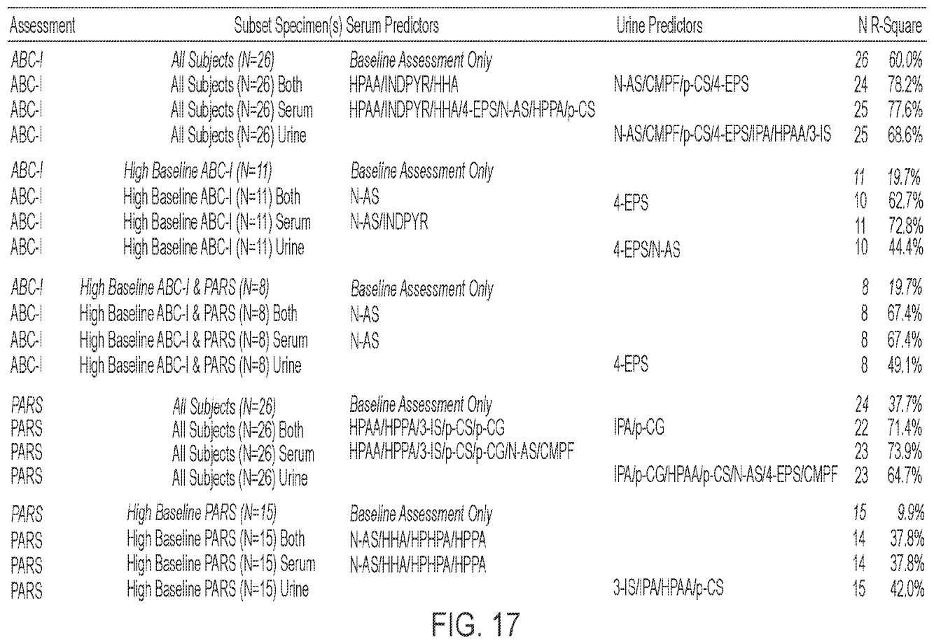

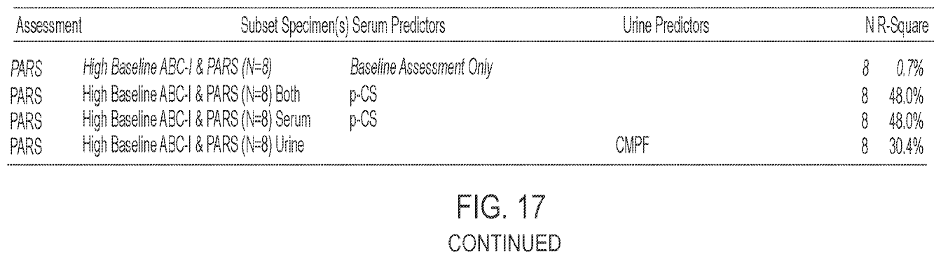

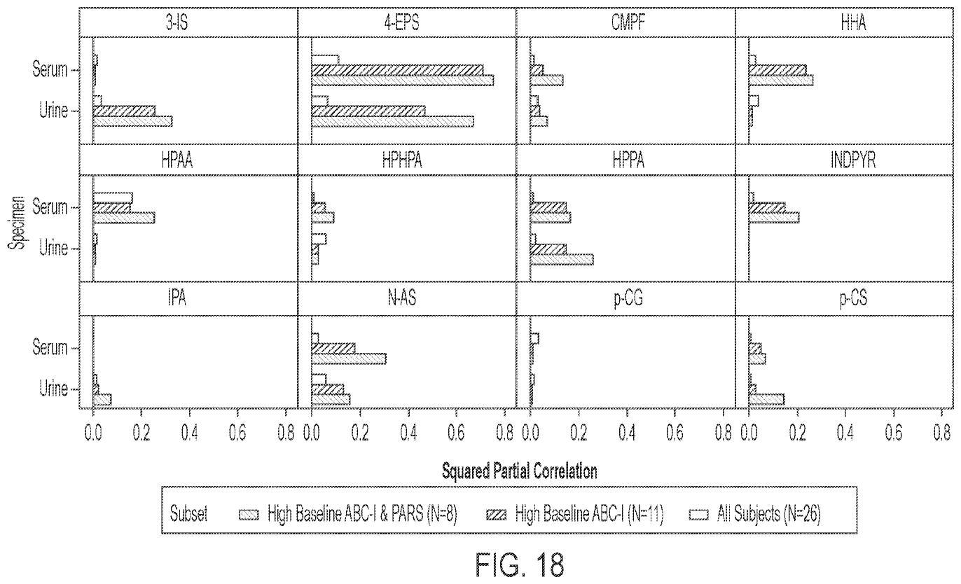

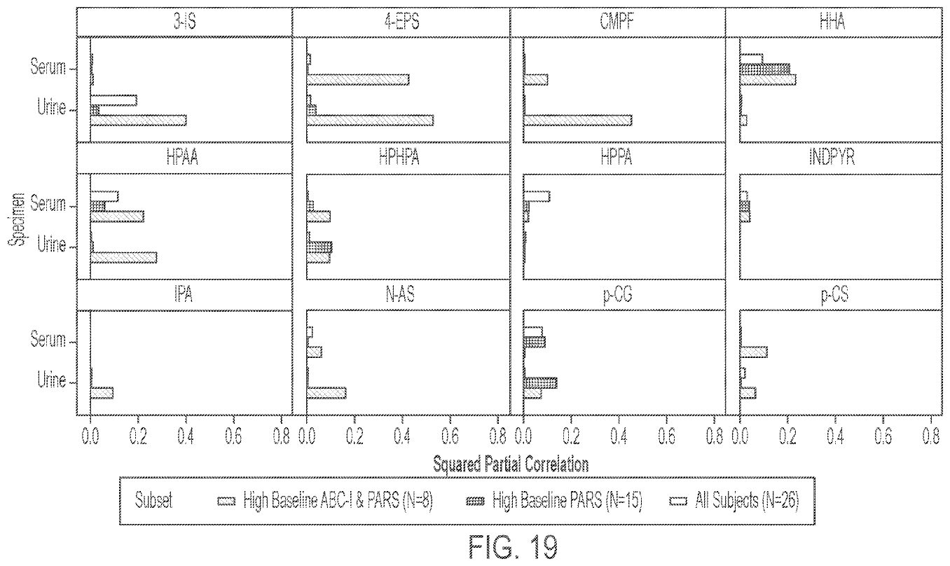

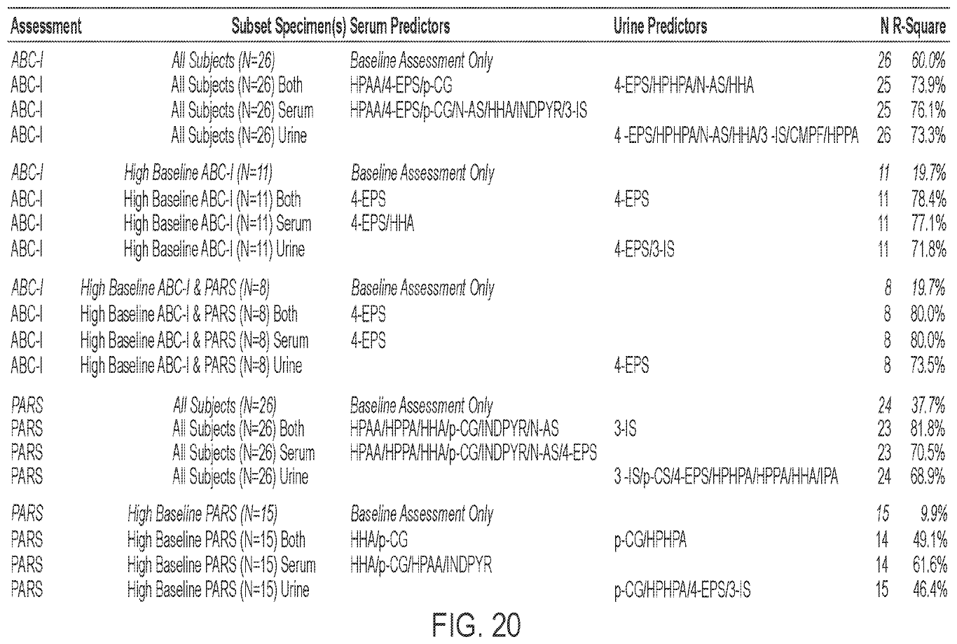

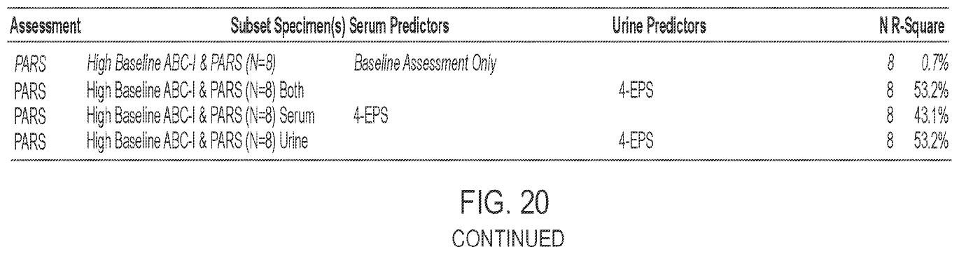

See FIGS. 12-14. FIGS. 12 and 13 show the correlation of individual metabolite levels at baseline (pre-treatment) with changes in irritability (ABC-I) score (FIG. 12), changes in anxiety (PARS) score (FIG. 13). As can be seen, some of these metabolites show good correlations to these score changes on an individual metabolite basis. For example, FIGS. 12 and 13 show high correlation of levels of serum and urine 4-EPS in subjects with high baseline irritability (ABC-I) and both high baseline irritability (ABC-I) and anxiety (PARS). FIG. 14 shows the correlations of different combinations of metabolites with these different subsets of subjects (all subjects, high baseline irritability, high baseline irritability and anxiety). For example, FIG. 14 shows several combinations of metabolites have r-squared correlation values >75%, which indicates a high correlation in this data set.

### 2. Composite Biomarkers of Change (e.g., Measuring Treatment Response)

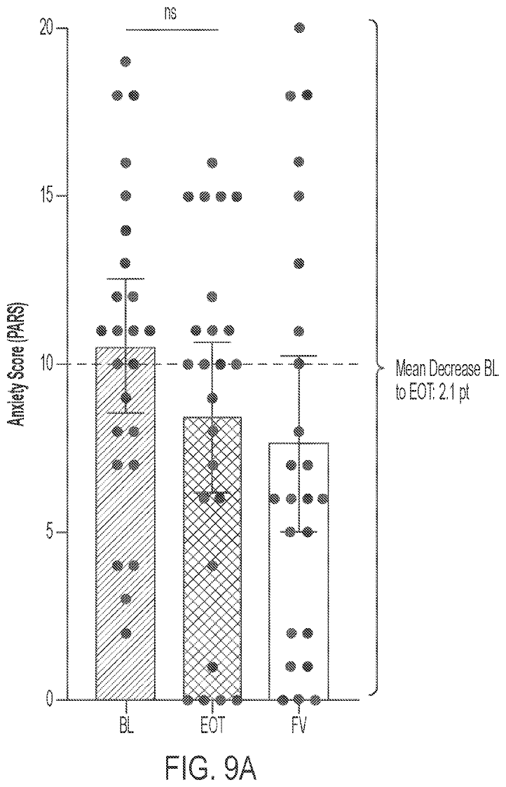

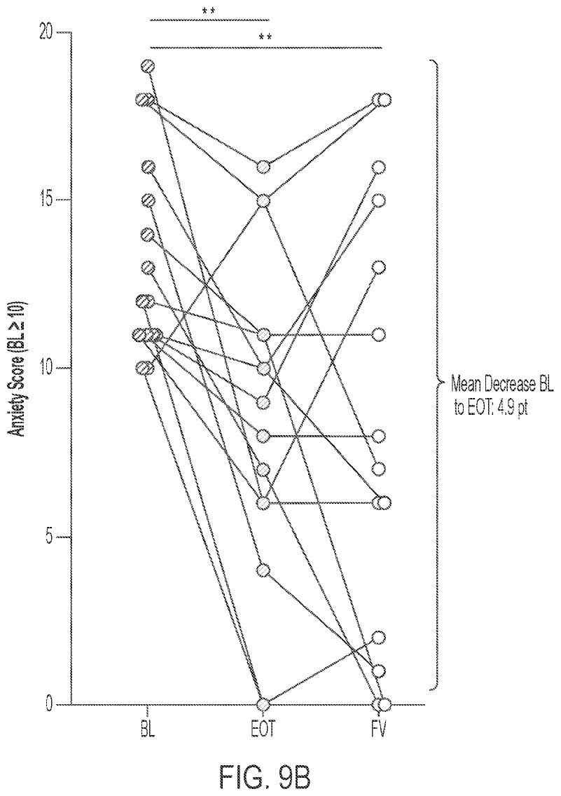

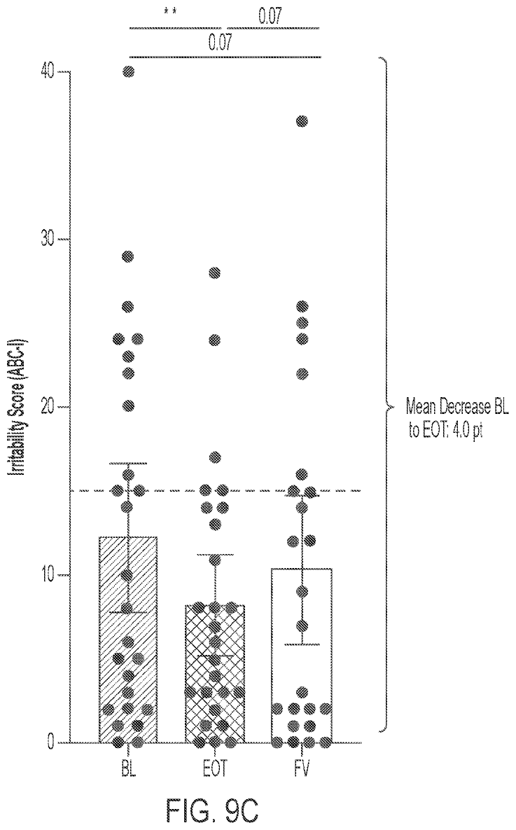

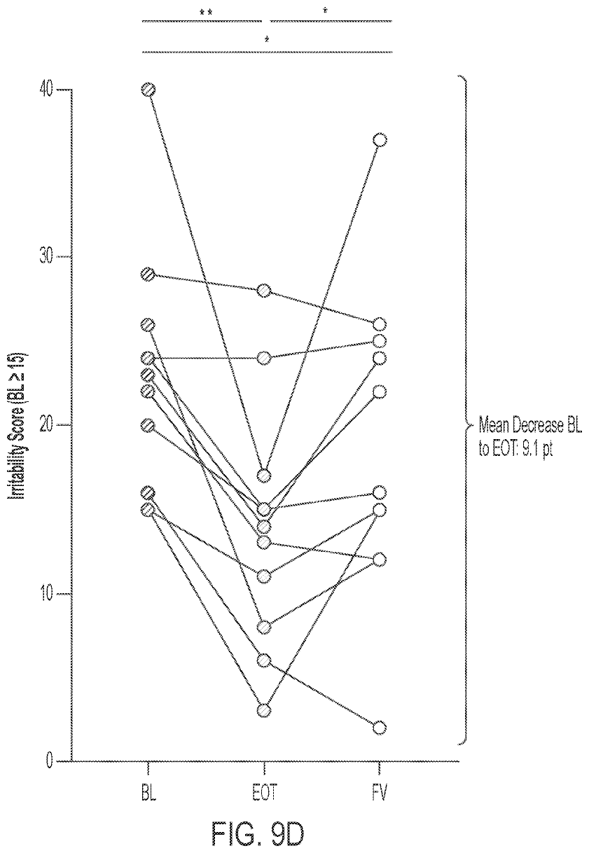

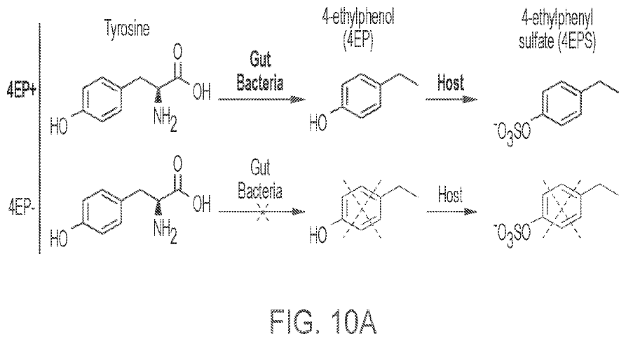

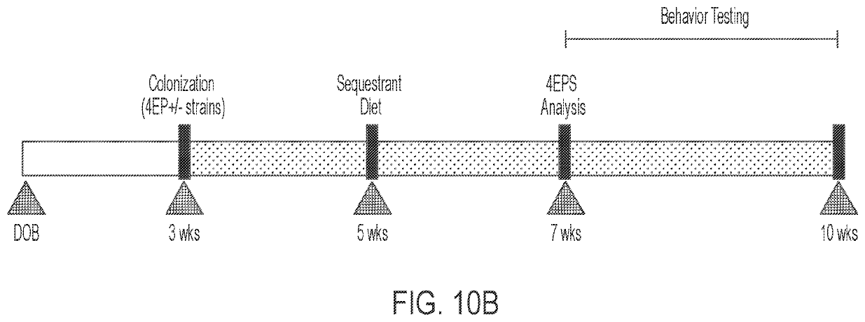

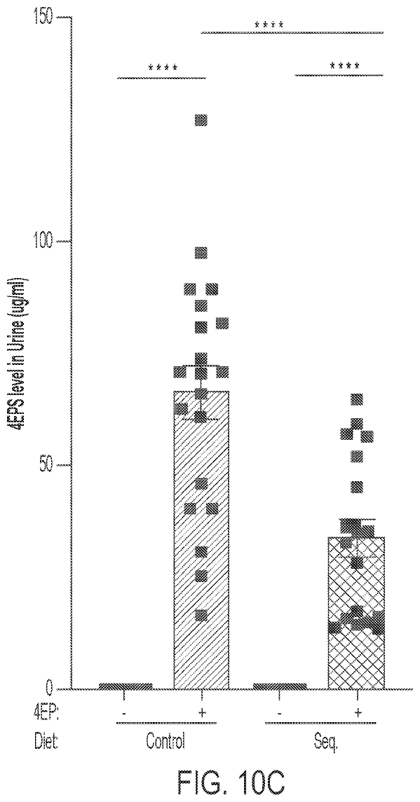

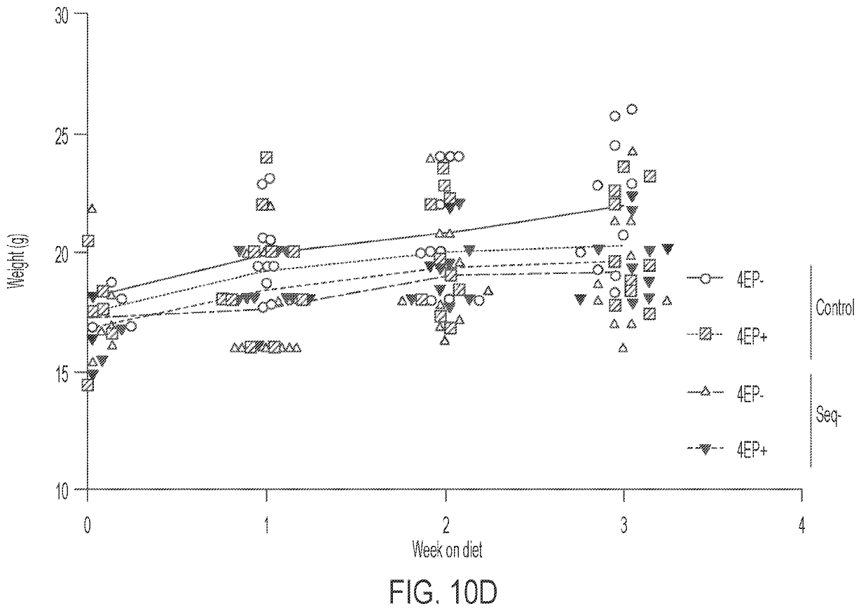

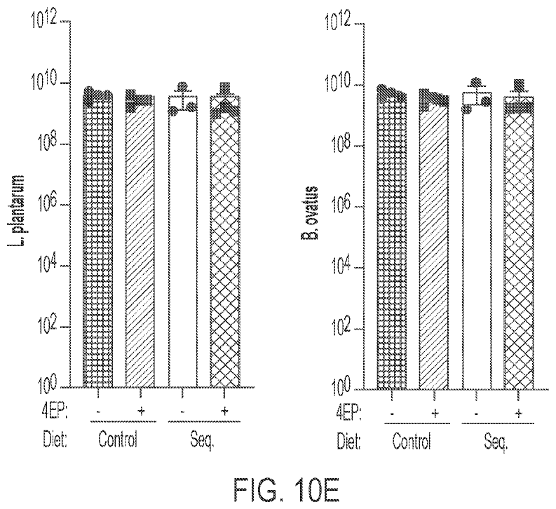

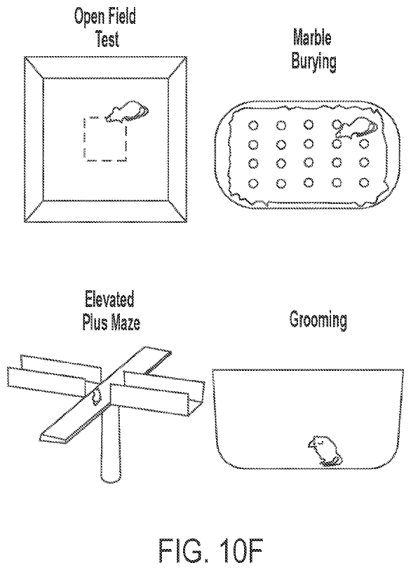

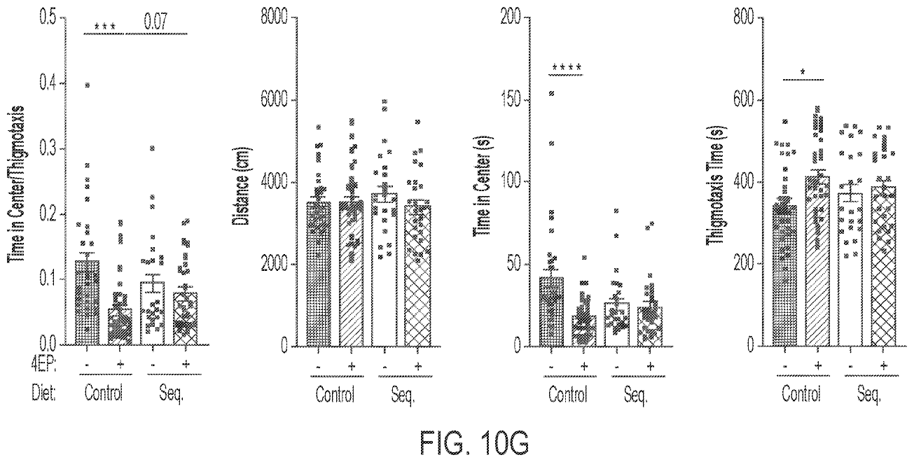

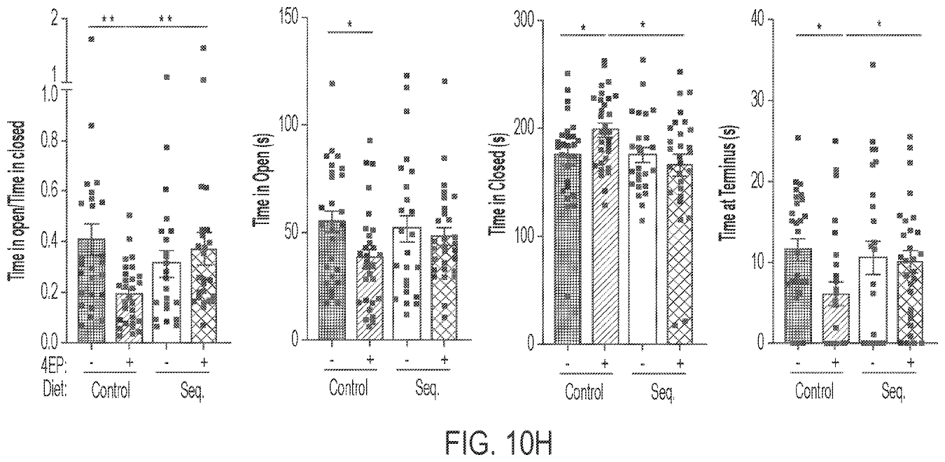

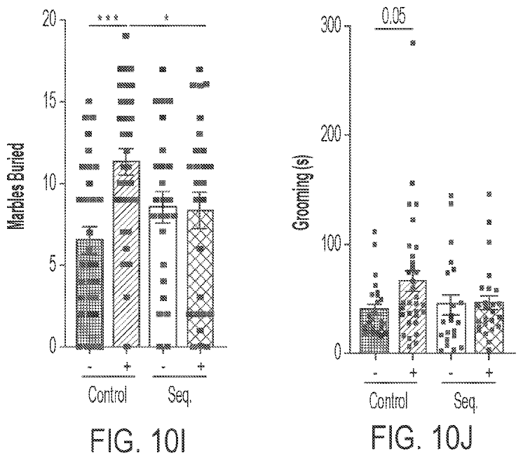

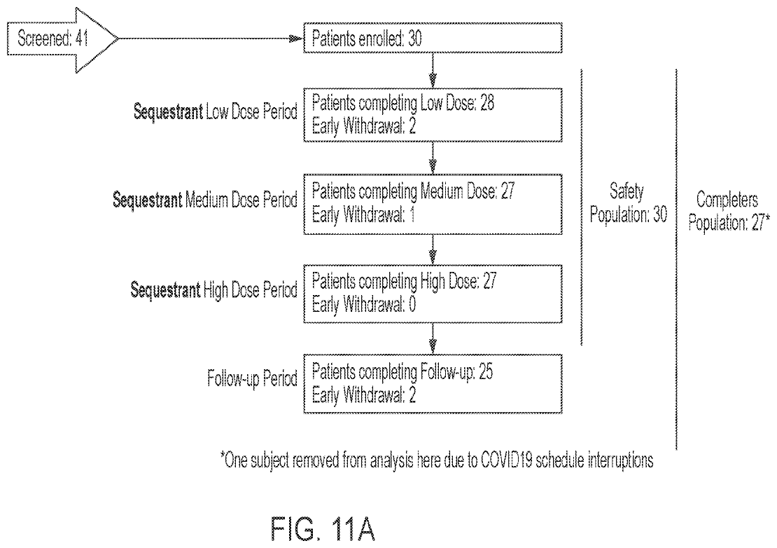

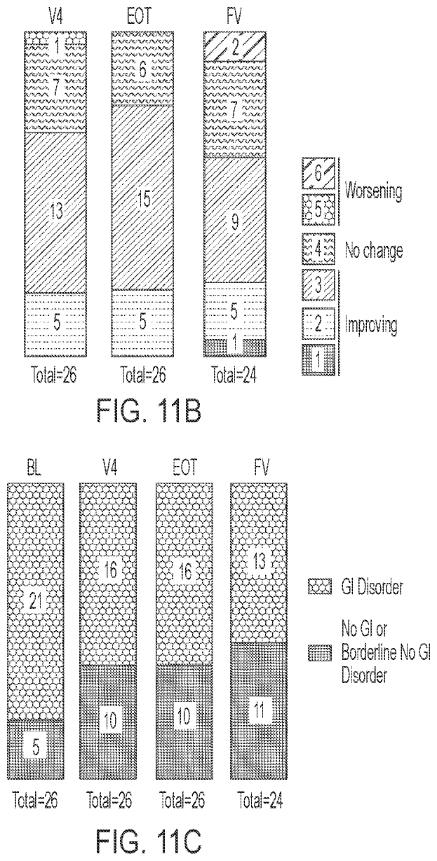

See FIGS. 9-11. FIGS. 9 and 10 shows show the correlation of change of individual metabolite levels after treatment with changes in irritability (ABC-I) score (page 1), changes in anxiety (PARS) score (page 2). Some of these metabolites show good correlations to these score changes on an individual metabolite basis. For example, FIG. 9 shows changes in serum and urine 4-EPS correlate strongly with changes in irritability (ABC-I) score. FIG. 11 shows the correlations of changes in different combinations of metabolites with changes in scores within these different subsets of subjects (all subjects, high baseline irritability, high baseline irritability and anxiety). For example, FIG. 11 shows changes in several combinations of metabolites have r-squared correlation values >70%, again, which indicates a high correlation in this data set.

### 3. Composite Biomarkers for Diagnosis

See FIGS. 12-14. This data can be used to make diagnoses. For example, those subjects with certain baseline levels of metabolites (e.g. 4-EPS in serum and urine, or HHA in serum) may be predicted to have high irritability given the high correlation value of change (improvement) in irritability. It could be inferred that those subjects must have measurable irritability, and hence could be diagnosed with irritability, in order to see an improvement in irritability.

4. Composite Biomarkers for Risk of Different Clinical Phenotypes (and/or Symptoms)

See FIGS. 9-14. This data can be used to predict risk of different clinical phenotypes or symptoms in a way that is explained under paragraph 3 above.

### Example 3

### Exploratory Analysis of Metabolite Biomarkers or Composite Biomarkers

An exploratory analysis was conducted to determine the nature of the relationship between change in metabolites from Baseline to Week 8 and change in assessment scores over that period, for subjects who participated in a placebo-controlled Phase 2b clinical trial in which AB2004 is tested. The wet methods and the statistical analysis described in Example 2 is used to analyze the Phase 2b data, and is adaptable to data sets of any size, and the predictive power should increase with larger data sets.

### Example 4

### 4A. Metabolite Binding Study

### Procedure for the Phosphate Buffer (Reduced Salt)

For preparation of 5× buffer, weigh the chemicals, 1.2 g of monobasic sodium phosphate and 1.6 g of dibasic sodium phosphate. Weigh 5 g of sodium chloride, 3.3 g of sodium acetate, 1.9 g of sodium propionate and 2.2 g of sodium butyrate.

To a 500 ml beaker, add 200 ml of purified water by the help of graduated cylinder and add the six weighed chemicals.

Place a stir bar and allow it to stir till the chemicals are completely dissolved.

Measure the pH of this mixture with pH meter and pH probe. Dropwise, with stirring add sodium hydroxide, (5 N) to adjust the pH to 7.2.

When pH 7.2 is obtained, add the mixture to a graduated cylinder and add purified water to make 1000 ml as a final volume of the 5× buffer.

### Procedure for Buffer Prep Used in the Experiment

Prepare Primary solutions of all three metabolites and Sodium Deoxycholate.

Weigh 200 mg of all the three metabolites and dissolve in water to make 200 mL volume to achieve the concentration of 1 mg/ml.

Weigh 1392.3 mg of Sodium deoxycholate and add 1391 mL of water to achieve the concentration of concentration of 1 mg/ml.

Add 696 ml of sodium deoxycholic acid primary solution into 2000 ml flask.

Add 100 ml of the Para cresol primary solution, 42 ml of Indole primary solution, 42 ml of 4-Ethyl phenol primary solution, 100 ml of Indoxyl Sulphate primary solution and 420 ml of Phosphate Buffer in the 2000 ml beaker and make up the volume to 2100 ml with water.

In a 50 mL centrifuge tube, weigh 20 mg of activated carbon particles. Repeat to generate triplicate samples for activated carbon particles and control. Label with name, replicate—1, 2 or 3.

For each triplicate, in each tube, add 20 mL of buffer. Note the starting time.

Screw the cap and place the tubes in incubator-shaker at 37 C and 60 revolutions.

Pull samples at 1, 3, 6 and 24 h into Omega 10K filter. Centrifuge to collect the filtrate. Place the filtrate in HPLC sample vial, ensuring that each adsorbent (labeled 1, 2 or 3) and each sample vial are labeled for the time point. Proposed label is name of adsorbent-duplicate #-timepoint.

### Quantitate by HPLC.

### Solutions Prepared According to Protocol:

Stock solution 1000 mL vol

| | gram | conc. Mg/mL |
|---|---|---|
| NaH₂PO₄ | 1.2 | 1.2 |
| Na₂HPO₄ | 1.6 | 1.6 |
| NaCl | 5 | 5 |
| Sodium | 1.9 | 1.9 |
| Propionate | | |
| Sodium | 2.2 | 2.2 |
| Butyrate | | |

### Binding Medium

### Results:

| Stock mL | Compound | final(ug/mL) |
|---|---|---|
| 100 | p-Cresol | 48 |
| 42 | 4-EP | 20 |
| 100 | Indoxyl sulfate | 48 |
| 42 | Indole | 20 |
| 696 | DCA | 331.429 |
| 420 | buffer | |
| | NaH₂PO₄ | 240 |
| | Na₂HPO₄ | 320 |
| | NaCl | 1000 |
| | Sodium | 380 |
| | Propionate | |
| | Sodium | 440 |
| | Butyrate | 500 |

| Indoxyl Sulfate | | | | | | | Conc. Calc. | |
|---|---|---|---|---|---|---|---|---|
| Sample | Time | | Peak Area | | | | | % |
| Name | point | | Prep-1 | Prep-2 | Prep-3 | Average | □g/mL | Remaining |
| Initial | t = 0 | | 756.92 | 759.69 | 785.24 | 767.28 | 48.23 | 100.0% |
| Activated | 1 | hrs | 711.20 | 720.68 | 747.43 | 726.44 | 45.65 | 94.7% |
| Carbon | 3 | hrs | 670.24 | 665.41 | 667.92 | 667.85 | 41.95 | 87.0% |
| | 6 | hrs | 629.97 | 641.60 | 648.89 | 640.15 | 40.20 | 83.4% |
| | 24 | hrs | 516.40 | 480.23 | 498.45 | 498.36 | 31.25 | 64.8% |

| p-Cresol | | | | | | | Conc. Calc. | |
|---|---|---|---|---|---|---|---|---|
| Sample | | | Peak Area | | | | | % |
| Name | Time | | Prep-1 | Prep-2 | Prep-3 | Average | □g/mL | Remaining |
| Initial | t = 0 | | 568.78 | 568.33 | 569.83 | 568.98 | 47.93 | 100.0% |
| Activated | 1 | hrs | 244.82 | 270.59 | 288.63 | 268.01 | 22.38 | 46.7% |
| Carbon | 3 | hrs | 147.51 | 124.74 | 107.04 | 126.43 | 10.36 | 21.6% |
| | 6 | hrs | 55.04 | 73.50 | 85.32 | 71.29 | 5.68 | 11.8% |
| | 24 | hrs | 16.80 | 14.33 | 15.81 | 15.65 | 0.95 | 2.0% |

| 4-EP | | | | | | | Conc. Calc. | |
|---|---|---|---|---|---|---|---|---|
| Sample | | | Peak Area | | | | | % |
| Name | Time | | Prep-1 | Prep-2 | Prep-3 | Average | □g/mL | Remaining |
| Initial | t = 0 | | 212.48 | 213.43 | 213.85 | 213.25 | 20.83 | 100.0% |
| Activated | 1 | hrs | 91.97 | 101.42 | 107.99 | 100.46 | 9.89 | 47.5% |
| Carbon | 3 | hrs | 54.52 | 44.88 | 39.15 | 46.18 | 4.62 | 22.2% |
| | 6 | hrs | 18.87 | 25.95 | 30.34 | 25.06 | 2.57 | 12.3% |
| | 24 | hrs | 2.27 | 1.67 | 1.92 | 1.96 | 0.33 | 1.6% |

| Indole | | | | | | Conc. Calc. | |
|---|---|---|---|---|---|---|---|
| Sample | Time | Peak Area | | | | | % |
| Name | point | Prep-1 | Prep-2 | Prep-3 | Average | □g/mL | Remaining |
| Initial | t = 0 | 287.42 | 289.12 | 288.46 | 288.34 | 21.30 | 100.0% |
| Activated | 1 hrs | 92.51 | 103.12 | 114.15 | 103.26 | 7.72 | 36.2% |
| Carbon | 3 hrs | 45.63 | 36.82 | 29.25 | 37.23 | 2.88 | 13.5% |
| | 6 hrs | 11.58 | 17.53 | 21.51 | 16.87 | 1.38 | 6.5% |
| | 24 hrs | 0.79 | 0.37 | 0.55 | 0.57 | 0.18 | 0.9% |

### 4B. Preparation of Formulations

The coating formulations, containing a dye, were cast in silicone molds, as described:

In a 15 mL centrifuge tube, 30 mg of Pectin (JRS Pharma and Batch Number: CL1925), was mixed in 1.92 mL water. The mixture was sonicated for 45 minutes until a homogenous dispersion was observed. This tube was labelled 043021-1 (Table 13).

In another 15 mL centrifuge tube, 30 mg of Soy Polysaccharides (JRS Pharma and Lot Number: P660011628X), was mixed in 1.92 mL water. The mixture was vortexed for 5 minutes to ensure sufficient mixing. This tube was labelled 043021-2 (Table 13).

In a 15 mL centrifuge tube, 30 mg of Guar Gum, (Spectrum Chemical MFG Corpand Lot Number: 2JD0301), was mixed in 1.92 mL water. The mixture was sonicated for 45-60 minutes until a homogenous dispersion was observed. This tube was labelled 043021-3 (Table 13).

To the mixtures mentioned in steps 1 to 3, Eudragit NM 30D (Evonik and Lot Number: C180862004) was added and the resulting dispersions were vortexed for 5 seconds.

To these dispersions, 150 μL of congo red solution was added and the dispersion was vortexed again for 5 seconds.

To these mixtures, Eudragit NM 30D (Evonik and Lot Number: C180862004), and congo red solution were mixed (Table 13).
To the tube labelled 043021-1, 0.9 mL of Eudragit NM 30D and 150 μL of congo red solution were added and the dispersion was vortexed for 2-3 minutes.
To the tube labelled 043021-2, 0.9 mL of Eudragit NM 30D and 150 μL of congo red solution were added and the dispersion was vortexed for 2 minutes.
To the tube labelled 043021-3, 0.9 mL of Eudragit NM 30D and 150 μL of congo red solution were added and the dispersion was vortexed for 2-3 minutes.

A single sided scotch tape was attached to the sticky side of the Grace-Bio silicone molds with 8-9 mm diameter×1.7 mm depth cavities.

These tape-attached molds were placed in a plastic container which were then placed within an incubation orbital shaker and the cavities in the molds were filled with the above dispersions.

All the above dispersions were vortexed for 10 seconds before filling them in the cavities of the molds.

The filled molds were dried in the incubation orbital shaker at 23 C and 175 rpm over the weekend.

TABLE 13
Formula table for quantities used of Eudragit NM
30D, Pectin, Soy Polysaccharides, and Guar Gum.

| | | | | | | 2% W/V |
|---|---|---|---|---|---|---|
| | | Soy | | | | Congo Red |
| | Pectin | Polysaccharides | Guar Gum | Water | Eudragit NM | Solution |
| Label | (mg) | (mg) | (mg) | (mL) | 30D (mL) | (μL) |
| 043021-1 | 30 | — | — | 1.92 | 0.9 | 150 |
| 043021-2 | — | 30 | — | 1.92 | 0.9 | 150 |
| 043021-3 | — | — | 30 | 1.92 | 0.9 | 150 |

### Observations

### See FIG. 21A-C

### 4C. Evaluation of Formulations

Effect of simulated GI environment on dissolution of the coating formulation was monitored by quantitating the amount of dye released. Example procedure and results are provided below:

Obtain 3 24-multi well plates and label the plates as follows:
Plate I: Standards Plate/Control (left half) and Pectin Films (right half)
Plate II: Guar Gum Films (left half) and Soy Polysaccharide Films (right half)
Plate III: Vegan Glaze Films (left half)

Cut the tea bags to create small pockets for the films to fit inside in each of the wells listed below
Plate I: Wells A4-A6, B4-B6 and C4-C6
Plate II: Wells A1-A6, B1-B6 and C1-C6
Plate III: Wells A1-A3, B1-B3 and C1-C3

Tape the tea bag pocket to the side wall of each well listed above and use forceps to insert one film per well

Once the film has been placed inside of the tea bag pocket, ensure the film is secure by taping the pocket's edges

Continue the process above until every pocket has a film inside

Before beginning experimentation, use a micropipette to pipette 2.4 mL of liquid per well based on the 5 tables below.

Using a screw driver, attach the polycarbonate box inside of the orbital shaker and place the 3 24-well plates inside

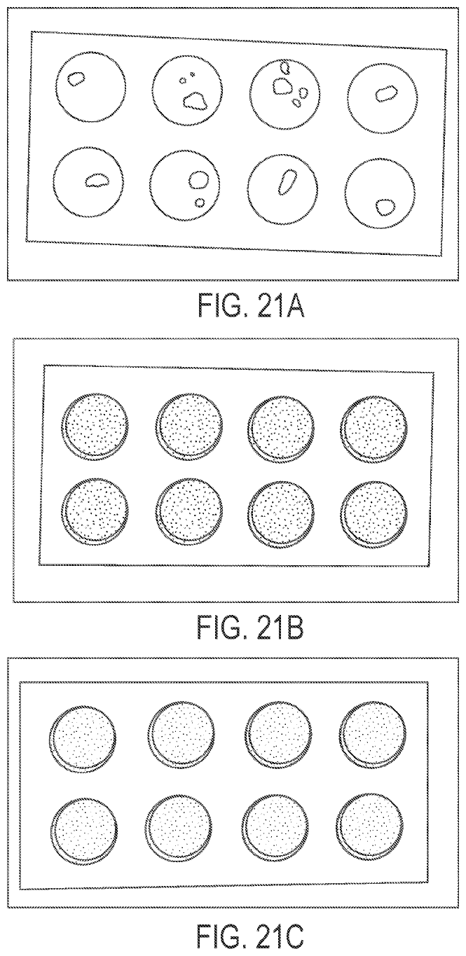

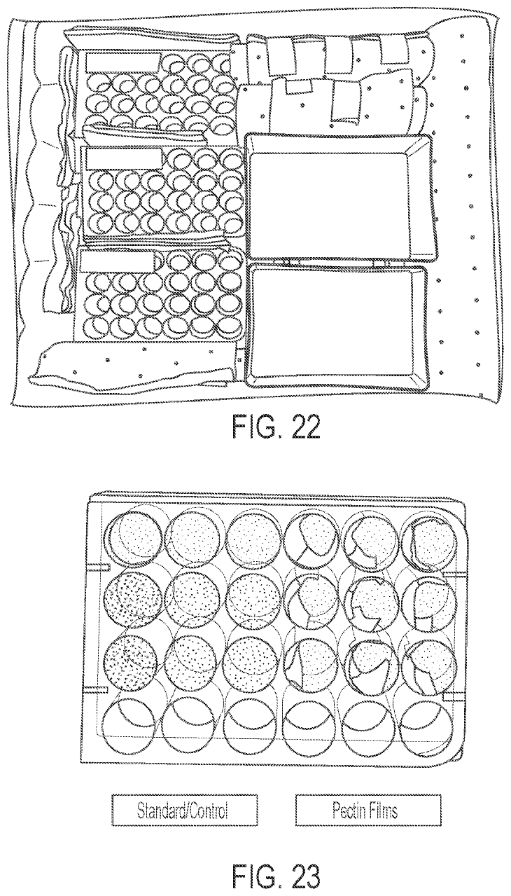

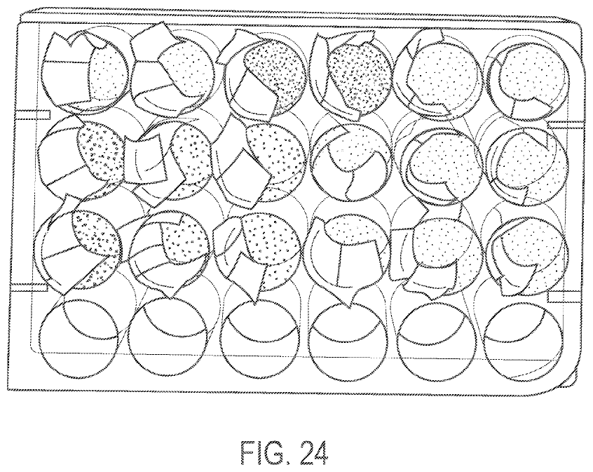

Use yellow soak pads and bubble wrap to ensure the well plates are secured inside of the polycarbonate box (see FIG. 22)

Once the well plates are secure, set the orbital shaker to 175 rpms and 37° C. Take the weight of each plate and absorbance readings using the spectrophotometer at each of the time points in the tables (0.5, 2, 4, 6, 24, 30 hours)

### Results:

TABLE 14
Plate I Absorbance Readings: Standards/Control:

| Time, | A1, NW | A2, NW | A3, NW | B1, SI | B2, SI | B3, SI | C1, C | C2, C | C3, C |
|---|---|---|---|---|---|---|---|---|---|
| h | 5 μg/mL | 10 μg/mL | 20 μg/mL | 5 μg/mL | 10 μg/mL | 20 μg/mL | 5 μg/mL | 10 μg/mL | 20 μg/mL |
| .5 | 0.403 | 0.657 | 1.305 | 0.847 | 1.355 | 1.75 | 0.267 | 0.445 | 0.814 |
| 2 | 0.386 | 0.661 | 1.337 | 1.055 | 1.855 | 1.855 | 0.274 | 0.474 | 0.899 |
| 4 | 0.411 | 0.686 | 1.262 | 1.131 | 1.538 | 2.306 | 0.331 | 0.534 | 0.944 |
| 6 | 1.404 | 0.672 | 1.248 | 1.179 | 1.589 | 2.015 | 0.628 | 0.791 | 1.121 |
| 24 | 0.386 | 0.653 | 1.224 | 1.198 | 1.594 | 2.035 | 2.049 | 1.373 | 2.64 |
| 30 | 0.371 | 0.600 | 1.081 | 1.227 | 1.573 | 2.017 | 2.099 | 2.103 | 2.628 |

TABLE 15
Plate I Absorbance Readings: Pectin:

| | A4, | A5, | A6, | B4, | B5, | B6, | C4, | C5, | C6, |
|---|---|---|---|---|---|---|---|---|---|
| Time, h | NW 4 | NW 5 | NW 6 | SI 4 | SI 5 | SI 6 | C 4 | C 5 | C 6 |
| .5 | 0.258 | 0.196 | 0.325 | 0.561 | 0.653 | 0.583 | 0.682 | 0.815 | 0.601 |
| 2 | 0.33 | 0.235 | 0.533 | 0.531 | 0.528 | 0.486 | 0.667 | 0.674 | 0.52 |
| 4 | 0.385 | 0.413 | 0.617 | 0.509 | 0.315 | 0.337 | 0.437 | 0.291 | 0.335 |
| 6 | 0.248 | 0.247 | 0.642 | 0.878 | 0.262 | 0.263 | 0.496 | 0.232 | 0.278 |
| 24 | 0.401 | 0.244 | 0.55 | 0.14 | 0.976 | 1.459 | 1.546 | 0.727 | 0.839 |
| 30 | 0.366 | 0.434 | 0.439 | 1.381 | 1.345 | 1.384 | 1.549 | 0.876 | 0.879 |

TABLE 16
Plate II Absorbance Readings: Guar Gum

| | A1, | A2, | A3, | B1, | B2, | B3, | C1, | C2, | C3, |
|---|---|---|---|---|---|---|---|---|---|
| Time, h | NW 1 | NW 2 | NW 3 | SI 1 | SI 2 | SI 3 | C 1 | C 2 | C 3 |
| .5 | 0.478 | 0.486 | 1.711 | 0.212 | 0.23 | 0.243 | 0.308 | 0.436 | 0.305 |
| 2 | 0.804 | 0.866 | 1.814 | 0.261 | 0.29 | 0.219 | 0.312 | 0.37 | 0.368 |
| 4 | 0.834 | 0.644 | 1.807 | 0.209 | 0.549 | 0.232 | 0.214 | 0.288 | 0.337 |
| 6 | 0.831 | 0.627 | 1.61 | 0.192 | 1.173 | 0.243 | 0.189 | 0.218 | 0.278 |
| 24 | 0.582 | 0.459 | 1.182 | 0.428 | 0.996 | 1.249 | 0.927 | 0.75 | 1.228 |
| 30 | 0.534 | 1.037 | 1.131 | 0.547 | 1.389 | 1.383 | 0.941 | 0.929 | 1.240 |

TABLE 17
Plate II Absorbance Readings: Soy Polysaccharide

| | A4, | A5, | A6, | B4, | B5, | B6, | C4, | C5, | C6, |
|---|---|---|---|---|---|---|---|---|---|
| Time, h | NW 4 | NW 5 | NW 6 | SI 4 | SI 5 | SI 6 | C 4 | C 5 | C 6 |
| .5 | 0.266 | 0.478 | 0.268 | 0.167 | 0.15 | 0.123 | 0.189 | 0.178 | 0.243 |
| 2 | 0.447 | 0.558 | 0.192 | 0.157 | 0.107 | 0.114 | 0.17 | 0.099 | 0.224 |
| 4 | 0.628 | 0.159 | 0.151 | 0.105 | 0.099 | 0.109 | 0.138 | 0.067 | 0.137 |
| 6 | 0.735 | 0.114 | 0.134 | 0.09 | 0.097 | 0.1 | 0.111 | 0.062 | 0.109 |
| 24 | 0.572 | 0.556 | 0.456 | 0.906 | 1.033 | 0.663 | 0.964 | 1.068 | 0.13 |
| 30 | 0.525 | 0.779 | 0.517 | 0.729 | 1.096 | 0.692 | 0.961 | 1.175 | 0.416 |

TABLE 18
Plate III Absorbance Readings: Vegan Glaze

| | A1, | A2, | A3, | B1, | B2, | B3, | C1, | C2, | C3, |
|---|---|---|---|---|---|---|---|---|---|
| Time, h | NW 1 | NW 2 | NW 3 | SI 1 | SI 2 | SI 3 | C 1 | C 2 | C 3 |
| .5 | 0.25 | 0.042 | 0.07 | 0.065 | 0.07 | 0.064 | 0.101 | 0.082 | 0.08 |
| 2 | 0.415 | 0.043 | 0.096 | 0.066 | 0.07 | 0.063 | 0.098 | 0.079 | 0.076 |
| 4 | 0.1 | 0.062 | 0.065 | 0.091 | 0.096 | 0.063 | 0.087 | 0.079 | 0.095 |
| 6 | 0.11 | 0.066 | 0.064 | 0.096 | 0.099 | 0.078 | 0.107 | 0.089 | 0.096 |
| 24 | 1.542 | 0.606 | 0.901 | 1.47 | 1.464 | 0.819 | 0.317 | 0.585 | 1.126 |
| 30 | 1.618 | 1.023 | 2.231 | 1.438 | 1.453 | 2.122 | 0.434 | 0.678 | 1.175 |

TABLE 19
Weight of Plates:

| Time, h | Plate I Weight (g) | Plate II Weight (g) | Plate III Weight (g) |
|---|---|---|---|
| 0 | 101.4 | 103 | 81.3 |
| .5 | 101.4 | 103 | 81.3 |
| 2 | 101.3 | 102.8 | 81.2 |
| 4 | 101.3 | 102.8 | 81.1 |
| 6 | 101.2 | 102.8 | 81.0 |
| 24 | 100.3 | 102.3 | 80.5 |
| 30 | 99.5 | 102.1 | 79.7 |

| | | Eudragit | | | Oleic |
|---|---|---|---|---|---|
| Type of | Formulation | FS30D | | Zein | Acid |
| Film: | # | (mL) | Polysaccharide | (mg) | (mg) |
| pH, | EuPZ1 | 20 | 20 | 54.54 | 5.454 |
| polysaccharide | EuPZ2 | 20 | 40 | 36.36 | 3.636 |
| and protease | EuPZ3 | 30 | 30 | 36.36 | 3.636 |
| | EuPZ4 | 40 | 20 | 36.36 | 3.636 |
| | EuPZ5 | 40 | 40 | 18.18 | 1.818 |

| Formulation # | Flozein (μL) | Oleic Acid (μL) | 85% EtOH |
|---|---|---|---|
| 010821-1 | 600 | 100 | 300 |
| 010821-2 | 622 | 67 | 311 |
| 010821-3 | 645 | 33 | 322 |

| Formulation | | Eudragit | |
|---|---|---|---|
| # | Surelease (mL) | FL30D (mL) | Water (mL) |
| 011221-1 | 1 | 0 | 1.5 |
| 011221-2 | 0 | 1 | 2 |

| Formulation | Zein Solution (10% Zein*) | Oleic Acid Solution |
|---|---|---|
| # | (μL) | (μL) |
| 011321-1 | 30 | 60 |
| 011321-2 | 65 | 130 |
| 011321-3 | 100 | 200 |

*Zein Solution = 100 mg/mL + 5M NaOH (increased pH >12.5)
Oleic Acid Solution = 50 mg Oleic Acid + 8 mL Water + .4 mL NaOH (5M) + 1.1 mL Water

| Formulation # | Zein Solution (μL) | Oleic Acid Solution (μL) |
|---|---|---|
| 011421-1 (3% OA) | 970 | 100 |
| 011421-2 (6.5% OA) | 935 | 216.6 |
| 011421-3 (10% OA) | 900 | 333.3 |

| Formulation # | Zein Solution (μL) | Oleic Acid Solution (μL) |
|---|---|---|
| 011521-1 (3% OA) | 970 | 1000 |
| 011521-2 (6.5%) | 935 | 2170 |
| 011521-3 (10%) | 900 | 3333 |

| Formulation # | Zein Solution (μL) | Oleic Acid Solution (μL) |
|---|---|---|
| 012021-1 (12% OA) | 880 | 4000 |
| 012021-2 (14% OA) | 860 | 4667 |
| 012021-3 (16% OA) | 840 | 5333 |

| | Eudragit FS | Eudragit FL | Water, | 1% Congo Red, |
|---|---|---|---|---|
| Formulation # | 30 D, mL | 30D, mL | mL | mL (stock) |
| 012721-1 | 0.24 | 0.06 | 0.6 | 0.1 |

| | 90% Zein, | | | |
|---|---|---|---|---|
| | 10% Oleic | | | 1% Congo |
| | Acid Solution, | Guar Gum, | Water, | Red, mL |
| Formulation # | mL | mg | mL | (stock) |
| 012721-2 | 10 | 200 | 9.8 | 0.1 |

| | | | | 90% Zein, | | Congo Red | Vol |
|---|---|---|---|---|---|---|---|
| Formulation | Eudragit | Eudragit | | 10% Oleic | | (20 mg/mL | diluent |
| # | L 30 D | FS 30 D | Alginate | Acid | Volume | stock), mL | (Water) |
| 021021-1 | 0.07 | 0.13 | 0.06 | 1.800 | 2.06 | 0.03 | 0.91 |
| 021021-2 | 0.07 | 0.13 | 0.12 | 1.200 | 1.52 | 0.03 | 1.45 |
| 021021-3 | 0.1 | 0.2 | 0.09 | 1.200 | 1.59 | 0.03 | 1.38 |
| 021021-4 | 0.13 | 0.27 | 0.06 | 1.200 | 1.66 | 0.03 | 1.31 |
| 021021-5 | 0.13 | 0.27 | 0.12 | 0.600 | 1.12 | 0.03 | 1.85 |
| 021021-6 | 0.07 | 0.13 | 0 | 2.400 | 2.60 | 0.03 | 0.37 |
| 021021-7 | 0.12 | 0.23 | 0 | 1.950 | 2.30 | 0.03 | 0.67 |
| 021021-8 | 0.17 | 0.33 | 0 | 1.500 | 2.00 | 0.03 | 0.97 |
| 021021-9 | 0.22 | 0.43 | 0 | 1.050 | 1.70 | 0.03 | 1.27 |
| 021021-10 | | | 0.06 | 2.400 | 2.46 | 0.03 | 0.51 |
| 021021-11 | | | 0.105 | 1.950 | 2.06 | 0.03 | 0.92 |
| 021021-12 | | | 0.15 | 1.500 | 1.65 | 0.03 | 1.32 |
| 021021-13 | | | 0.195 | 1.050 | 1.25 | 0.03 | 1.73 |
| | | Eudragit | | | | | |
| | | NM 30 D | | | | | |
| 021021-15 | | 0.3 | 0.09 | 1.200 | 1.59 | 0.03 | 1.38 |

| Film | Eudragit FS | Eudragit FL | 2% Congo Red, mL |
|---|---|---|---|
| Formulation # | 30 D, mL | 30 D, mL | (stock) |
| 022421-1 | 4.0 | 1.0 | 1.0 |

| Formulation | Eudragit L | Eudragit | 10% | 90% Zein, | 2% Congo | Vol |
|---|---|---|---|---|---|---|
| # | 30 D, mL | FS 30 D, | Pectin, g | 10% Oleic | Red, mL | Diluent |
| | | mL | | Acid, mL | | (Water) |
| EuPZ-1-315 | 0.07 | 0.13 | 0.6 | 1.800 | 0.22 | 0.18 |
| EuPZ-2-315 | 0.07 | 0.13 | 1.2 | 1.200 | 0.22 | 0.18 |
| EuPZ-3-315 | 0.10 | 0.2 | 0.9 | 1.200 | 0.22 | 0.38 |
| EuPZ-4-315 | 0.13 | 0.27 | 0.6 | 1.200 | 0.22 | 0.58 |
| EuPZ-5-315 | 0.13 | 0.27 | 1.2 | 0.600 | 0.22 | 0.58 |
| EuZ-1-315 | 0.07 | 0.13 | 0 | 2.400 | 0.22 | 0.18 |
| EuZ-2-315 | 0.12 | 0.23 | 0 | 1.950 | 0.22 | 0.48 |
| EuZ-3-315 | 0.17 | 0.33 | 0 | 1.500 | 0.22 | 0.78 |
| EuZ-4-315 | 0.22 | 0.43 | 0 | 1.050 | 0.22 | 1.08 |
| PZ-1-315 | 0 | 0 | 0.600 | 2.400 | 0.22 | 0 |
| PZ-2-315 | 0 | 0 | 1.050 | 1.950 | 0.22 | 0 |
| PZ-3-315 | 0 | 0 | 1.500 | 1.500 | 0.22 | 0 |
| PZ-4-315 | 0 | 0 | 1.950 | 1.050 | 0.22 | 0 |
| | | Eudragit | | | | |
| | | NM30D | | | | |
| NMPZ-1-315 | | 0.3 | 0.9 | 1.200 | 0.22 | 0.38 |
| NMPZ-2-315 | | 0.2 | 1.2 | 1.200 | 0.22 | 0.18 |
| NMPZ-3-315 | | 0.1 | 1.350 | 1.350 | 0.22 | 0 |

| | Eudragit | | 90% Zein, | 2% Congo |
|---|---|---|---|---|
| Formulation | NM30D, | 10% | 10% Oleic | Red, mL |
| # | mL | Pectin | Acid, mL | (stock) |
| NMPZ-4-317 | 0.1 | 0 | 2.700 | 0.22 |
| NMPZ-5-317 | 0.1 | 2.700 | 0 | 0.22 |

| | | Eudragit | 10% Pectin | 2% Congo | Diluent |
|---|---|---|---|---|---|
| Formulation | | NM 30D, | Dispersion, | Red, mL | (Water), |
| # | Layer | mL | mg | (stock) | mL |
| CP-1-319-L1 | Inner | 0 | 3000 | 0.22 | 0 |
| E-1-319-L2 | Outer | 1 | 0 | 0 | 2 |

| | | Eudragit | 10% Pectin | 2% Congo | Diluent |
|---|---|---|---|---|---|
| Formulation | | NM 30D, | Dispersion, | Red, mL | (Water), |
| # | Layer | mL | mg | (stock) | mL |
| CP-2-319-L1 | Inner | 0 | 3000 | 0.22 | 0 |
| EP-2-319-L2 | Outer | 0.9 | 270 | 0 | 1.83 |

| Formulation # | Aquazein, mL | Vegan Glaze, mL | 10% Pectin, mg |
|---|---|---|---|
| AZP-1-423 | 2.4 | 0 | 60 |
| VGP-1-423 | 0 | 2.4 | 60 |

| | | | Soy | Guar | 2% Congo | |
|---|---|---|---|---|---|---|
| Formulation | Eudragit | Pectin, | Polysaccharides, | Gum, | Red, mL | Water, |
| # | NM30D, mL | mg | mg | mg | (stock) | mL |
| 043021-1 | 0.9 | 30 | 0 | 0 | 0.15 | 1.92 |
| 043021-2 | 0.9 | 0 | 30 | 0 | 0.15 | 1.92 |
| 043021-3 | 0.9 | 0 | 0 | 30 | 0.15 | 1.92 |

| | 10% w/v | 10% w/v | 10% w/v | 10% w/v | Vegan | 2% Congo | |
|---|---|---|---|---|---|---|---|
| Formulation | polypropylene | Tween | Levulinic | Pectin, | Glaze, | Red, mL | Water, |
| # | glycol, mL | 80, mL | Acid, mL | mg | mL | (stock) | mL |
| 050321-1 | 0.6 | 0.6 | 0 | 0 | 0.5 | 0.22 | 0.8 |
| 050321-2 | 0 | 0.6 | 0 | 0 | 0.5 | 0.22 | 0.8 |
| 050421-3 | 0.6 | 0.6 | 0 | 0 | 0.5 | 0.22 | 6 |
| 050421-4 | 0.6 | 0.6 | 0 | 300 | 2 | 0.22 | 5.7 |
| 050421-5 | 0 | 0.6 | 0.6 | 300 | 2 | 0.22 | 5.7 |
| 050421-5 (II) | 0 | 0.6 | 0.6 | 300 | 2 | 0.22 | 5.7 |
| 050421-6 | 0 | 0 | 0.6 | 300 | 2 | 0.22 | 6.3 |

| Formulation # | NM30D, mL | 2% Congo Red, mL (stock) |
|---|---|---|
| 051221-1 | 3.0 | .22 |

| | Eudragit | Pectin | | 2% Congo | |
|---|---|---|---|---|---|
| Formulation | NM30D, | Powder, | Water, | Red, mL | Heat |
| # | mL | mg | mL | (stock) | Treatment? |
| 052021-1 | 1.8 | 60 | 3.84 | 0.3 | No |
| 052021-3 | 1.8 | 60 | 3.84 | 0.3 | Yes (Pectin) |
| 052021-5 | 1.8 | 60 | 3.84 | 0.3 | Yes |
| | | | | | (NM30D |
| | | | | | and Pectin) |

| | | | | 5% w/v | | | |
|---|---|---|---|---|---|---|---|
| | | | | Tween 80 | | 2% Congo | |
| Formulation | Vegan | Levulinic | 10% w/v | Solution, | Water, | Red, mL | Heat |
| # | Glaze, mg | Acid, mg | Pectin, mg | mL | mL | (stock) | Treatment? |
| 052021-2 | 2000 | 60 | 30 | 0.6 | 0.1 | 0.15 | No |
| 052021-4 | 2000 | 60 | 0 | 0.6 | 0.4 | 0.15 | No |
| 052121-6 | 2000 | 60 | 30 | 0.6 | 0.1 | 0.15 | Yes |
| 052121-7 | 2000 | 60 | 0 | 0.6 | 0.4 | 0.15 | Yes |

| | | | | | 2% Congo | |
|---|---|---|---|---|---|---|
| Formulation | Vegan | Levulinic | 10% | 85% | Red, mL | Water, |
| # | Glaze, mg | Acid, mg | Pectin, mg | Hydroalcohol | (stock) | mL |
| 052621-1 | 3051.0 | 91.5 | 508.5 | 1017.0 | 0.3 | 1032.0 |
| 052621-2 | 2757.3 | 82.7 | 1034.5 | 919.1 | 0.3 | 906.3 |
| 052621-3 | 2456.1 | 73.7 | 1578.9 | 818.7 | 0.3 | 772.7 |
| 052621-4 | 2142.8 | 64.3 | 2142.8 | 714.3 | 0.3 | 635.9 |
| 052621-5 | 1816.7 | 54.5 | 2725.0 | 605.6 | 0.3 | 498.3 |

| | | | | | 2% Congo | |
|---|---|---|---|---|---|---|
| Formulation | Vegan | 10% | Palmitic | | Red, mL | Water, |
| # | Glaze, mg | Pectin, mg | Acid, mg | DBS, mg | (stock) | mL |
| 060221-2 | 3051.0 | 508.5 | 228.8 | 0 | 0.3 | 1.912 |
| 060221-3 | 3051.0 | 508.5 | 0 | 160.2 | 0.3 | 1.912 |

| | | Starch-Butanol |
|---|---|---|
| Formulation # | Eudragit NM30D, mL | Complex*, mL |
| 060821-1 (20% Starch) | 5 | 2.7 |
| 060821-2 (30% Starch) | 7.5 | 2.33 |
| 061121-6 (40% Starch) | 10 | 2 |

*Starch-Butanol Complex: 4 g Amylo N-400 + 2 g Butanol and 88 g Water

| | Eudragit | Starch- | 2% Congo | |
|---|---|---|---|---|
| Formulation | NM30D, | Butanol | Red, mL | Heat |
| # | mL | Complex, mL | (stock) | Treatment? |
| 061421-1 | 0 | 6.00 | 0.22 | Yes |
| (100% S-B) | | | | |
| 061421-2 | 0 | 6.00 | 0.22 | No |
| (100% S-B) | | | | |
| 061421-3 | 6.00 | 0 | 0.22 | No |
| (0% S-B) | | | | |

*Films above were made with starch-butanol complex prepared on Jun. 13, 2021 and kept in the refrigerator overnight

| Formulation | Starch-Butanol | 2% Congo Red, mL | Heat |
|---|---|---|---|
| # | Complex, mL | (stock) | Treatment? |
| 061521-1 (100% | 6.00 | 0.22 | Yes |
| S-B) | | | |
| 061521-2 (100% | 6.00 | 0.22 | No |
| S-B) | | | |

*Films above were made with starch-butanol complex that was freshly prepared

| | Eudragit | Starch- | 2% Congo | |
|---|---|---|---|---|
| Formulation | NM30D, | Butanol | Red, mL | Heat |
| # | mL | Complex, mL | (stock) | Treatment? |
| 061621-1 | 2.0 | 10.0 | 0.60 | No |
| (40% S-B) | | | | |
| 061621-2 | 0.67 | 7.5 | 0.41 | No |
| (60% S-B) | | | | |
| 061621-3 | 0.33 | 10.0 | 0.52 | No |
| (80% S-B) | | | | |
| 061621-4 | 0 | 6.0 | 0.30 | No |
| (100% S-B) | | | | |

| | | Vegan Glaze & | 2% Congo |
|---|---|---|---|
| Formulation | Degradation | 85% EtOH | Red, mL |
| # | Method | Solution, mL | (stock) |
| 062221-A | Initial Sample | 6.0 | 0.3 |
| 062221-B | Acidification & | 6.0 | 0.3 |
| | Neutralization | | |
| 062221-C | Acidification, | 6.0 | 0.3 |
| | Heat Treatment & | | |
| | Neutralization | | |
| 062221-D | Homogenization | 6.0 | 0.3 |

*Starting Zein Solution (pre-degradation methods/initial sample): 25 g Vegan Glaze diluted with 12.5 mL of 85% EtOH

| | Vegan Glaze | | | 2% Congo |
|---|---|---|---|---|
| Film | & 85% EtOH | Levulinic | | Red, mL |
| Formulation # | Solution, mL | Acid, mg | | (stock) |
| 062321-1 | 6.0 | 300 | (5%) | 0.3 |
| 062321-2 | 6.0 | 600 | (10%) | 0.3 |
| 062321-3 | 6.0 | 900 | (15%) | 0.3 |

*Starting Zein Solution (pre-degradation methods/initial sample): 25 g Vegan Glaze diluted with 12.5 mL of 85% EtOH

| | | Vegan Glaze & | 2% Fluorescein- |
|---|---|---|---|
| Film | Degradation | 85% EtOH | Dextran, mL |
| Formulation # | Method | Solution, mL | (stock) |
| 062921-A | Initial Sample | 6.0 | 0.3 |
| 062921-B | Acidification & | 6.0 | 0.3 |
| | Neutralization | | |
| 062921-C | Acidification, | 6.0 | 0.3 |
| | Heat Treatment & | | |
| | Neutralization | | |
| 062921-D | Homogenization | 6.0 | 0.3 |

*Starting Zein Solution (pre-degradation methods/initial sample): 25 g Vegan Glaze diluted with 12.5 mL 85% EtOH

| | Vegan Glaze | | 2% Congo | Heat |
|---|---|---|---|---|
| | & 85% EtOH | Levulinic | Red, mL | Treatment: |
| Formulation # | Solution, mL | Acid, mg | (stock) | (°, t) |
| 070721-1A | 6.0 | 0 | 0.3 | 80° C., .5 h |
| 070721-1B | 6.0 | 0 | 0.3 | 80° C., 2 h |
| 070721-1C | 6.0 | 0 | 0.3 | 80° C., 4 h |
| 070821-1D | 6.0 | 0 | 0.3 | 80° C., 8 h |
| 070721-2A | 6.0 | 50 | 0.3 | 80° C., .5 h |
| 070821-3A | 6.0 | 0 | 0.3 | 80° C., .5 h |

*Vegan Glaze & 85% EtOH Solution:
070721-1A, 1B, 1C, and 070821-1D, 3A: 20 g Vegan Glaze and 10 mL 85% EtOH
070721-2A: 5 g Vegan Glaze and 2.5 mL 85% EtOH

### 4D. Methods of Making Controlled-Release Compositions and Formulations

In some embodiments, multiple-layer systems of the present disclosure are generated by preparing in the mold film/disk of the bottom layer, allowing it to dry, and then adding the next layer. The process is continued until the desired number of layers are generated. During dissolution studies, the disk is protected on the bottom layer so only the top layer of the film is exposed to the dissolution conditions.

In some embodiments, one or more of the optimized formulations of the present disclosure, from the above screening matrix, are coated onto a therapeutic agent core using particle coating technology (for example, Wurster coating apparatus), using formulations and processes known to one skilled in the art.

In some embodiments, a coated therapeutic agent is evaluated using the dissolution conditions described herein. Dissolution testing and evaluation are performed using a USP Apparatus 3, utilizing simulated “fed stomach” buffers with enzymes; simulated small intestinal buffers with enzymes; simulated ascending colon (AC) buffers with enzymes; simulated transverse colon (TC) buffers with enzymes; and simulated descending colon (DC) buffers with enzymes. The composition of the buffers is tabulated:

| | Concentration | | Fed-small | | | | | | | |
|---|---|---|---|---|---|---|---|---|---|---|
| Component | Fed-Gastric | | intestine | | AC | | TC | | DC | |
| Sodium chloride | 237 | mM | | | | | | | | |
| Acetic acid | 17 | mM | | | | | | | | |
| Sodium acetate | 30 | mM | | | | | | | | |
| Pepsin, USP/NF | 1.5 | mg/mL | | | | | | | | |
| Deoxycholic acid | | | 3.64 | mg/mL | 0.364 | mg/mL | 0.364 | mg/mL | 0.364 | mg/mL |
| Na₂HPO₄ | | | 25 | mM | 10 | mM | 10 | mM | 10 | mM |
| NaH₂PO₄ | | | 25 | mM | 10 | mM | 10 | mM | 10 | mM |
| Pancreatin, | | | 10 | mg/mL | 10 | mg/mL | 10 | mg/mL | 10 | mg/mL |
| USP/NF | | | | | | | | | | |
| Pectinase/amylase | | | | | 1 | mg/mL | 0.5 | mg/mL | | |
| Bacterial protease | | | | | | | 0.5 | mg/mL | 1 | mg/mL |

| | | | | | |
|---|---|---|---|---|---|
| HCl/NaOH QS to | pH 5 | pH 6.5 | pH 5.75 | pH 6.25 | pH 6.75 |

AC: Ascending colon; TC: Transverse colon; DC: descending and sigmoidal colon

As described above, the dissolution apparatus allows for successive exposures of a coated therapeutic agent core to simulated GI buffers representing successive GI-compartments, encountered by orally-administered therapeutic agents, drugs or medications. When a dissolution test is performed in buffers containing p-cresol, and the p-cresol remaining post-exposure is quantitated, observation of a reduction in p-cresol level signifies availability of a sequestrant core to bind molecules in the buffer (believed to be due to dissolution/breakdown of whole or part of the therapeutic agent's coating or coatings). Formulations showing utilization of less than 10% of the binding capacity of the sequestrant in the pre-colonic buffers, but showing >50% of the binding capacity (compared to uncoated sequestrant core, subjected to the same conditions) utilized in colonic conditions, are considered for the next stage of evaluation. Selected pharmaceutical compositions for oral administration comprising coated therapeutic agent formulations can be further evaluated in a simulator of the human gastrointestinal processes, such as the ProDigest SHIME® apparatus. Formulations that demonstrate utilization of less than 10% of the binding capacity of the sequestrant core in the pre-colonic buffers, but demonstrating >50% of the binding capacity (compared to uncoated sequestrant, subjected to the same conditions) utilized in colonic conditions, are considered for the next stage of evaluation.

Selected coated therapeutic formulations can also be evaluated in suitable animal models, such as non-human primate, dog, mini-pig, etc. The formulations showing lack of drug-drug interaction with an immediate release formulation but showing urine or serum metabolite reduction equivalent to or greater than metabolite reduction observed for uncoated sequestrant, administered under similar conditions, are considered for the next stage of evaluation.

Selected coated therapeutic formulations can also be evaluated in humans. The formulations showing lack of drug-drug interaction with an immediate release formulation but showing urine or serum metabolite reduction equivalent to or greater than metabolite reduction observed for uncoated sequestrant, administered under similar conditions, are considered for the next stage of evaluation.

### (Formulation Example 5) In Vivo Evaluation Test (1): Influence on Blood Level of Simultaneously Administered Drug (Methylphenidate)

The methylphenidate-containing spherical carbon adsorbent capsule preparation can be orally administered to Cynomolgus monkeys one capsule per animal (5 animals in total). Blood can be collected before oral administration and 0.5, 1, 2, 4, 8, and 24 hours after administration, the plasma methylphenidate (active metabolite levels of methylphenidate) level can be measured, and the average of 5 animals can be summarized in a graph. As a comparative control, a combination capsule preparation can be used that could be produced in the same manner, except that size-5 capsules not coated with an enteric polymer can be filled with a spherical carbon adsorbent. Also, in order to measure the plasma methylphenidate level when methylphenidate is administered alone, one size-0 capsule filled solely with methylphenidate can be administered. It was shown that spherical carbon adsorbent when administered simultaneously with methylphenidate, which is often prescribed for treating ADD or ADHD subjects, reduces the blood level thereof, whereas the pharmaceutical preparation of the present disclosure, even when administered simultaneously with methylphenidate, enables the blood level to be maintained at the same level as a level attained when the therapeutic agent or drug is administered singly.

### (Formulation Example 6) In Vivo Evaluation Test (2): Influence on Blood Level of Simultaneously Administered Drug (Concerta)

Formulation Example 6 can be performed in the same manner as Formulation Example 5 by using the Concerta-containing spherical carbon adsorbent capsule preparation. Note that the dosage of Concerta could be 10 mg, and the plasma level of Concerta can be measured.

It can be shown that a spherical carbon adsorbent when administered simultaneously with Concerta, which is often prescribed for ADD or ADHD subjects, could reduce the blood level, whereas the capsule preparation of the present disclosure, even when administered simultaneously with Concerta, can enable the blood level to be maintained at the same level as a level attained when the therapeutic agent or drug is administered singly.

### (Formulation Example 7) in Vivo Evaluation Test (3): Influence on Plasma Uremic Toxin Levels or Bacterial Metabolite Levels

In this test, the granule preparation (Formulation Example 7) coated, for example, with Eudragit S100 as an enteric polymer to a ratio of 40% in terms of polymer solids relative to activated carbon particles could be used. Dosing solutions can be prepared by suspending the granule preparation and uncoated spherical carbon adsorbent each in an amount of 0.3 g in terms of the weight of spherical carbon adsorbent in 10 mL of a 1.25% tragacanth solution. 10 mL each of the dosing solutions could be orally administered per 1 kg of male rats, or an alternate rodent or non-rodent species, at 11 weeks old (N=5). The rats could be fasted after administration, blood could be collected from the caudal vein before oral administration and 1, 2, 4, and 6 hours after administration, the plasma level of p-cresol sulfate (PCS), which is one of the uremic toxins or bacterial metabolites, can be measured, and the extent of reduction of plasma PCS (D Plasma PCS, mg/dL) from the time before oral administration can be calculated. The average.+/−.standard error of each group at each time point can be graphed. The granule preparation of the present disclosure (Formulation Example 7) could show a greater reduction of plasma p-cresol sulfate level than uncoated spherical carbon adsorbent.

### (Formulation Example 8) In Vivo Evaluation Test (4): Dosage of Spherical Carbon Adsorbent and Influence on Plasma Uremic Toxin Levels or Bacterial Metabolite Levels

In this test, among the granule preparations produced in Example 3, the granule preparation (Formulation Example 12) coated with Eudragit S100 and L100 (3:1) as enteric polymers to a ratio of 30% in terms of polymer solids relative to activated carbon particles was used. Dosing solutions can be prepared by suspending the granule preparation and uncoated spherical carbon adsorbent each in an amount of 100 mg or 300 mg in terms of the weight of spherical carbon adsorbent in 10 mL of a 1.25% tragacanth solution. 10 mL each of the dosing solutions can be orally administered per 1 kg of SD(Slc:SD) rats (N=8 to 9). The rats can be fasted after administration, and 2 hours after oral administration, 1 mL of a probenecid solution (50 mg/mL) can be intravenously administered per 1 kg of the rats. Blood can be collected from the vein before oral administration, before intravenous administration, and 1, 2, 3, and 4 hours after intravenous administration, the plasma level of p-cresol sulfate (PCS), which is one of the uremic toxins or bacterial metabolites, can be measured, and the extent of reduction of plasma PCS (D plasma PCS, mg/dL) from the time before oral administration can be calculated. The granule preparation of the present disclosure (Formulation Example 12) showed an identical reduction of plasma p-cresol sulfate level in a dosage ⅓ of uncoated spherical carbon adsorbent.

### (Formulation Example 9) In Vivo Evaluation Test (5): Dosage of Spherical Carbon Adsorbent and Influence on Plasma Uremic Toxin Levels or Bacterial Metabolite Levels (Non-Rodent Animal)

In this test, among the granule preparations produced in Example 3, the granule preparation (Formulation Example 12) coated with Eudragit 5100 and L100 (3:1) as enteric polymers to a ratio of 30% in terms of polymer solids relative to activated carbon particles can be used. Gelatin capsules (No. 12 (¼ oz)) can be filled respectively with the granule preparation and uncoated spherical carbon adsorbent so as to be 100 mg or 300 mg in terms of the weight of spherical carbon adsorbent per 1 kg of Beagle dogs and could be orally administered. 1 hour after oral administration, 2.5 mL of a methylphenidate solution (20 mg/mL) can be orally administered per 1 kg of Beagle dogs. 2 hours after oral administration of spherical carbon adsorbent, the dogs can be fed (DS-A, 250 g/dog) again. Blood was collected from the vein before feeding and 2, 4, 6, and 8 hours after feeding, the plasma level of p-cresol sulfate (PCS), which is one of the uremic toxins or bacterial metabolites, was measured, and the extent of reduction of plasma PCS (D plasma PCS, mg/dL and D plasma PCS AUC (0 to 8 h), mgh/dL) from the time before feeding can be calculated. The average.+/−.standard error of each group at each time point can be graphed, and D plasma PCS AUC from before feeding (0 hours) to 8 hours can be determined. The granule preparation of the present disclosure (Formulation Example 12) might show an identical reduction of plasma p-cresol sulfate level in a dosage ⅓ of uncoated spherical carbon adsorbent.

### (Formulation Example 10) In Vivo Evaluation Test (6): Influence on Blood Uremic Toxin Levels or Bacterial Metabolite Levels (when the Type of Spherical Carbon Adsorbent and the Type/Amount of Coating of Enteric Polymer are Changed)

In this test, among the granule preparations produced in Example 3, the granule preparations (Formulation Examples 15, 16, 12, 19, 22, and 13) coated with an enteric polymer to a ratio of 10 to 30% in terms of polymer solids relative to spherical carbon adsorbent can be used. Dosing solutions can be prepared by suspending the granule preparations and uncoated spherical carbon adsorbent each in an amount of 100 mg in terms of the weight of spherical carbon adsorbent in 10 mL of a 1.25% tragacanth solution. 10 mL each of the dosing solutions was orally administered per 1 kg of rat or other rodent species (N=6 to 19). The rats can be fasted after administration, and 2 hours after oral administration, 1 mL of a methylphenidate solution (50 mg/mL) can be intravenously administered per 1 kg of the rats. Blood can be collected from the vein before oral administration, before intravenous administration, and 1, 2, 3, and 4 hours after intravenous administration, the plasma level of p-cresol sulfate (PCS), which is one of the uremic toxins or bacterial metabolites, can be measured, and the extent of reduction of plasma PCS (D plasma PCS, AUC (˜2 h to 4 h)) from the time before oral administration can be calculated. The ratio to the solvent control group (a 1.25% tragacanth solution administered group) being can be calculated, and the average of each group can be graphed. The granule preparations of the present disclosure (Formulation Examples 15, 16, 12, 19, 22, and 13) can show a greater reduction of plasma p-cresol sulfate level than uncoated spherical carbon adsorbent.
Pharmaceutical preparation (Type of carbon ratio vs adsorbent/coating, dissolution pH, amount) N control Control 19 1.00 Activated Carbon 19 0.47
Formulation Example 15 (Activated Carbon, 6-0.36 Eudragit_L30D55, pH 5.5, 10%)
Formulation Example 16 (Activated Carbon, 6-0.02 Eudragit_L30D55, pH 5.5, 30%)
Formulation Example 12 (Activated Carbon, 6 0.04 Eudragit_S100:L100=3:1, pH 6.75, 30%)
Formulation Example 19 (Activated Carbon, 7 0.33 Eudragit_S100, pH 7.0, 30%)
Formulation Example 22 (Activated Carbon, 6 0.35 HPMC-AS-HG, pH 6.5, 17%)
Formulation Example 13 (Activated Carbon, 6 0.20 Eudragit_S100:L100=3:1, pH 6.75, 30%)

### (Formulation Example 11) In Vivo Evaluation Test (7): Verification of Exposed State of Spherical Carbon Adsorbent Surface of a Pharmaceutical Composition for Oral Administration Preparation of the Present Disclosure, in Small Intestine, Large Intestine (Descending Colon)

In this test, among the granule preparations produced in Example 3, the granule preparation (Formulation Example 7) coated with Eudragit S100 as an enteric polymer to a ratio of 40% in terms of polymer solids relative to activated carbon particles can be used. 0.3 g of the granule preparation in terms of weight can be weighed and suspended in 10 mL of a 1.25% tragacanth solution to prepare a dosing solution. After 10 mL of the dosing solution can be orally administered per 1 kg of male rats at 11 weeks old, the rats can be fasted, then euthanized with carbon dioxide after 1 and 4 hours, and subjected to laparotomy to observe the state of spherical carbon adsorbent in the digestive tract. At 1 hour after administration, spherical carbon adsorbent could be present in the small intestine. This could appear grayish white, and it might be verified by observation with the naked eye that the coating was maintained. At 4 hours after administration, spherical carbon adsorbent can be present in the large intestine, in the descending colon, the coating dissolved, and spherical carbon adsorbent returned to black. It could be verified that activated carbon particles, which is the content, could be exposed. Accordingly, it could be shown that the enteric polymer does not dissolve in the small intestine but dissolves in the large intestine, in the descending colon, and activated carbon particles, which is the content, could be exposed in the descending colon.

### (Formulation Example 12) In Vivo Evaluation Test (8): Influence on Blood Level of Simultaneously Administered Drug (Methylphenidate) of Granule Preparation Wherein Spherical Carbon Adsorbent is Coated with Enteric Polymer

A granule preparation produced in the same manner as in Formulation Example 12 of Example 3 (Eudragit S100 and L100 (3:1) as enteric polymers) was used. One size-0 capsule filled with 171.+/−.4 mg of the granule preparation coated to a ratio of 30% relative to activated carbon particles and 0.5 mg of methylphenidate (0.5 mg of methylphenidate content obtained by grinding a 5 mg methylphenidate tablet) could be orally administered to Cynomolgus monkey (6 animals in total). Blood could be collected before oral administration and 0.5, 1, 2, 4, 8, and 24 hours after administration, the plasma methylphenidate level can be measured, and the average of 6 animals can be summarized in a graph. Eight days after the administration described above, one size-0 capsule could be filled with granular activated carbon particles (120.+/−.2 mg) not coated with an enteric polymer and 0.5 mg of methylphenidate (0.5 mg of methylphenidate content obtained by grinding a 5 mg methylphenidate tablet) was orally administered as a comparative control and, similarly, blood was collected to measure the plasma methylphenidate level, and the average of 6 animals can be summarized in a graph. Furthermore, in order to measure the plasma methylphenidate level when methylphenidate is administered singly, 12 days after the administration described above, one size-0 capsule filled with 0.5 mg of methylphenidate (0.5 mg of methylphenidate content obtained by grinding a 5 mg methylphenidate tablet) was orally administered and, similarly, blood can be collected to measure the plasma methylphenidate level, and the average of 6 animals was summarized in a graph. The results of total 3 times can be graphed.

It can be shown that spherical carbon adsorbent when administered simultaneously with methylphenidate, which is often prescribed for treating subjects with ADD or ADHD, reduces the blood level thereof, whereas the pharmaceutical compositions for oral administration preparations of the present disclosure, even when administered simultaneously with methylphenidate, enables the blood level to be maintained at the same level as a level attained when the therapeutic agent, or drug, is administered singly.

### (Formulation Example 13) Adsorption Test: Adsorption Performance Evaluation, of p-Cresol, by Granules Wherein a Spherical Carbon Adsorbent is Coated with a Colon-targeting Polymer Layer

The granule preparations of Formulation Example 12 and Formulation Examples 13 and 15 to 22 corresponding to 50 mg in terms of spherical carbon adsorbent cam be weighed and used in the test.

Test solutions having a pH of 1.2 (United States Pharmacopeia (USP) or Japanese Pharmacopoeia dissolution test 1st solution JP1) and a pH of 7.5 (Mcllvaine buffer) can be used. The test solutions can be adjusted such that the concentration of p-cresol, which is an adsorbate, is 40 mg/L. p-Cresol absorbance can be measured using a dissolution apparatus by a paddle method at 200 rpm in 500 mL of a test solution at a UV measurement wavelength of 246 nm (the test time is 2 hours at a pH of 1.2 and 6 hours at a pH of 7.5). In reference to the JIS activated carbon test, the amount of p-cresol adsorbed on 1 g of spherical carbon adsorbent can be calculated.

The results of the test performed using Formulation Example 12 and Formulation Examples 13 and 15 to 22 can be graphed. In the adsorption test using p-cresol, it can be verified that the pharmaceutical preparations adsorbed p-cresol in an amount of less than 50 mg/g in 2 hours in a test solution having a pH of 1.2, and adsorbed p-cresol in an amount of 80 mg/g or more in 6 hours in a test solution having a pH of 7.5. These results could show that pharmaceutical composition for oral administration preparations, coated with a colon-targeting polymer of the present disclosure, remain in a coated state or form, until reaching the target site (the descending colon) and thus function to maintain the intrinsic adsorption performance without being reduced, and demonstrate adsorption performance for the first time, after reaching the target site.

p-Cresol Amount of Adsorption Test (mg/g)

| Number | coating | pH 1.2 | pH 7.5 |
|---|---|---|---|
| Formulation Example 12 | 30% | 10.9 | 173.2 |
| Formulation Example 13 | 30% | 20.5 | 357.0 |
| Formulation Example 15 | 10% | −0.6 | 143.3 |
| Formulation Example 16 | 30% | −4.1 | 191.0 |
| Formulation Example 17 | 60% | 23.6 | 156.1 |
| Formulation Example 18 | 100% | 3.6 | 142.1 |
| Formulation Example 19 | 30% | 14.1 | 209.0 |
| Formulation Example 20 | 30% | 0.2 | 124.0 |
| Formulation Example 21 | 10% | 10.1 | 115.3 |
| Formulation Example 22 | 17% | 11.4 | 168.3 |

The pharmaceutical composition for oral administration preparations of the present disclosure do not affect, impair or otherwise adversely impact the blood levels of a concomitant therapeutic agent or drug, even when simultaneously administered, or co-administered with one or more concomitant therapeutic agents or drugs, and possess greater toxic substance adsorbability properties in a living subject or living body compared to a conventional spherical carbon adsorbent. The skilled artisan should appreciate that the volume of pharmaceutical composition, when administered, can be reduced, and the amount of water consumption can also be reduced. In some embodiments, pharmaceutical compositions for oral delivery preparations are effective for reducing blood uremic toxins or bacterial metabolites, inhibiting or ameliorating behavioral symptoms of a neurological disorder such as autism, autism spectrum disorder (ASD) and associated pathologies including intestinal hyperpermeability or leaky gut improving uremic symptoms, delaying dialysis initiation, or protecting renal functions in a subject with CKD.

### 4E. In Vitro Model of Immediate and Targeted Release

In a preliminary experiment, an activated carbon particle adsorbent was pre-exposed to a simulated upper gastro-intestinal tract, before exposure to metabolites in simulated colonic conditions. The simulation of the upper GI tract included simulated digested food as well as bile and pH changes (digestive enzymes were not added). Three different exposure conditions were used in this experiment. The buffer solution #1 mimicked the “fed” conditions of the stomach, which consisted of the commercially-available product “Ensure® Clear” liquid nutritional drink to represent, or model, a digested meal and, sodium chloride to provide ionic strength. 10 mg of activated carbon particles was weighed in each of three 250 ml bottles. These were exposed to buffer solution #1 for 105 mins, on an incubator shaker, at 250 rpm and 37° C. Later, the bottles were taken out of the incubator shaker and small intestinal conditions were simulated by addition of sodium taurocholate (bile salt). pH was increased to 6.5 by addition of sodium hydroxide. The bottles were placed in the incubator shaker for 345 mins. Then, this buffer solution was filtered out through a buffon cap, retaining the activated carbon particles in the bottles. The buffer solution #2 was prepared to simulate the condition of the ascending and transverse colon by adding sodium acetate and sodium deoxycholate and acetic acid to adjust to pH 6. 60 ml of the buffer solution #2 was added to the bottles and activated carbon particles were exposed to the same incubator conditions for 960 mins. In order to mimic the descending colon, buffer solution #3 was prepared with sodium acetate, sodium deoxycholate and 3 metabolites. The concentrations of the metabolites were: p-cresol at 6 g/ml, p-cresol and 4-EP at 2 g/ml. The buffer solution #2 was removed from the bottles and buffer solution #3 was added. Along with these three bottles exposed to the GI condition, three more bottles containing activated carbon particles unexposed to buffer solutions #1 or #2 were introduced and buffer solution #3 was added. The six bottles were placed in an incubator shaker having 250 rpm for 1500 mins. 1 ml samples were pipetted at all timepoints (1, 2, 5, 24, 25 hours). An HPLC assay was used to quantitate the adsorbent remaining in solution. Measured metabolite adsorption kinetics were significantly decreased when the activated carbon particles were pre-exposed to simulated upper-GI conditions. For the first 5 h, activated carbon particles pre-exposed to upper GI conditions bound only half as much metabolites as activated carbon particles that were freshly introduced to colonic buffer with metabolites. At the 24 h timepoint, freshly introduced activated carbon particles still bound more metabolites, but the difference was negligible. Faster removal of metabolites is envisioned to help drive down the metabolite concentrations in the GIT.

The results demonstrated potential improvements in binding for such targeted release (TR) over the simulated immediate release (IR) administration of activated carbon particles. Furthermore, for in vivo applications, due to the presence of a multitude of adsorbates available in the upper GIT, the benefits of targeted release achieved could be greater compared to that of a non-coated composition or system.

### 4F. Testing Adsorption of p-Cresol by Activated Carbon

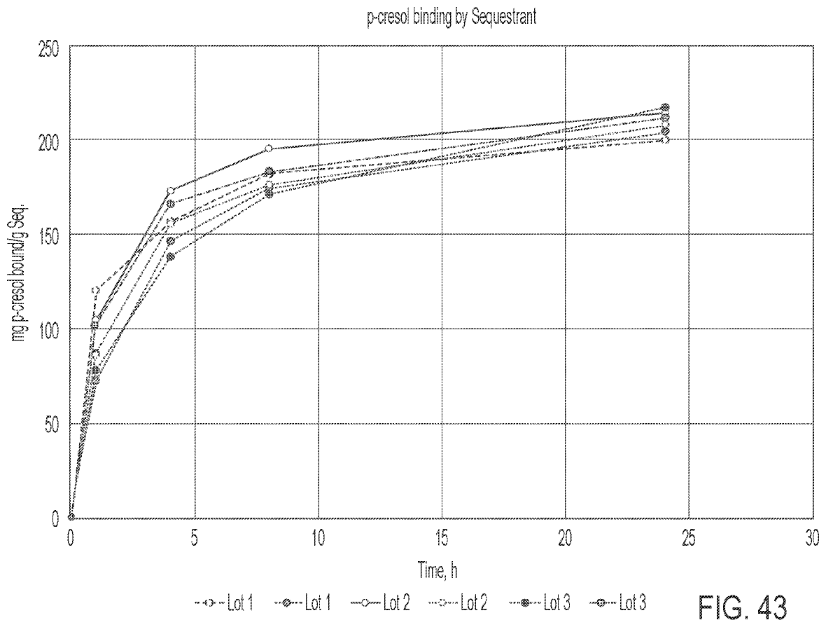

In some embodiments, the pharmaceutical preparation of the present disclosure is tested for p-cresol adsorption. For example, an amount corresponding to about 75 mg of the therapeutic agent is tested in a USP dissolution apparatus using 150 mL of a pH 7.2 phosphate buffer having a p-cresol concentration of 67 ug/mL at 37 C. Buffer samples are pulled at 0, 1, 4, 8 and 24 h and p-cresol concentration is quantitated by HPLC. P-cresol bound to activated carbon particles is calculated from decrease in p-cresol concentration in the buffer. FIG. 43 shows duplicate p-cresol binding obtained for 3 lots. Under the test conditions, at 24 h time point, not less than 178 mg and not more than 242 mg of p-cresol is bound per g of activated carbon particles.

The adsorption method can be miniaturized by decreasing the proportion of the buffer as well as activated carbon particles.

(Adsorption test method): Evaluations are made using a sample (for example, an amount corresponding to about 50 mg in terms of the weight of the therapeutic agent when the pharmaceutical composition preparation is a granule preparation containing the therapeutic coated with an enteric polymer, one tablet when the pharmaceutical preparation is a tablet preparation, one capsule when the capsule contents are coated with polymers described earlier).

A test solution having a pH of about 1.2 (such as the United States Pharmacopeia (USP) or Japanese Pharmacopeia dissolution test 1st solution JP1) or a pH of about 7.5 (such as Mcllvaine buffer) is used. The test solution is adjusted such that the concentration of p-cresol, which is an adsorbate, is 40 mg/L. The p-cresol absorbance is measured using a dissolution apparatus by a paddle method at 200 rpm in 500 mL of a test solution at 246 nm of UV measurement wavelength (the test time is 2 hours at a pH of 1.2 and 6 hours at a pH of 7.5). In reference to the JIS activated carbon test, the amount of p-cresol adsorbed on 1 g of spherical carbon adsorbent is calculated.

For the pharmaceutical composition preparation of the present disclosure, because the therapeutic agent is isolated from the environment of the digestive tract until the target site is reached, the composition functions to deliver and maintain an optimal intrinsic adsorption performance without being reduced, impaired, or diminished, and delivers such optimal adsorption performance of the therapeutic agent to the desired target site.

### 4G. Methods of Making and Assessing Controlled-Release Compositions

The present disclosure provides that the optimal region in the GIT to release a therapeutic agent core is determined using “Apparatus 3”, which is defined as comprising the USP Apparatus 3, a reciprocating cylinder apparatus, utilized for mimicking reported values for parameters such as time, temperature, pH, as well as short chain fatty acid, bile acid, protein and metabolite concentrations for the ascending, transverse, and descending and sigmoidal colon regions. A therapeutic agent core is introduced in a buffer representing the composition of ascending colon and after representative time frame, progressed to buffer representing transverse and then, descending colon. Furthermore, in a second set, the therapeutic agent core is introduced in buffer representing transverse colon and then progressed to buffer containing descending colon. In the third set, the therapeutic agent core is introduced into buffer containing descending and sigmoidal colon. Net metabolite removal by each of these introductions of a therapeutic agent core is quantitated to determine optimal colonic site for introduction of the therapeutic agent core. Alternatively, or in addition to USP Apparatus 3, a person skilled in the art can use one or more of a variety of dissolution apparatuses. For example, one can use one or more dissolution apparatus listed in the United States Pharmacopeia (USP), European pharmacopeia (EP), Japanese pharmacopeia (JP), or, by using other experimental techniques, such as ProDigest SHIME®, TNO's TIM-1 or TIM-2, Pion, Inc. μFLUX™, MacroFLUX™ or inFORM™, or similar techniques.

The SHIME® GI model is a unique scientifically validated dynamic model of the complete gastrointestinal tract to study physicochemical, enzymatic and microbial parameters in the gastrointestinal tract in a controlled in vitro setting. The model consists of five reactors which sequentially simulate the stomach (acid conditions and pepsin digestion), small intestine (digestive processes) and the 3 regions of the large intestine, i.e., the ascending, transverse and descending colon (microbial processes). Careful control of the environmental parameters in these reactors delivers complex and stable microbial communities which are highly similar in both structure and function to the microbial community in the different regions of the human colon. This model can be used to study the metabolic fate of food, microbial and pharmaceuticals compounds over a period of several weeks.

The present disclosure provides results of screening of the targeted release formulations by forming dye-incorporated film/disk embodiments of the formulations disclosed. Stability of the formulations to different GI conditions (pH, enzymes) and rate of dissolution of a film component of the formulation is monitored using a plate reader, quantitating for dye released/solubilized. The following formulations are planned for initial screening (the numerical figures in the table represent percentages of the components comprising the composition):

In some embodiments, the coating of a therapeutic agent with a colon-targeting polymer can utilize a variety of widely-available procedures; among the procedures, techniques and devices available for the coating of fine particles are particular apparatuses used in coating fine particles, which include but are not limited to, a composite fluidized bed granulator coater, a Wurster fluidized (or fluid) bed granulator coater, a tumbling fluidized bed granulator coater, or a fluidized bed granulator coater, among others.

Tablet preparations and the like can be coated with a colon-targeting polymer by widely-used apparatuses and techniques designed for film coating and the like.

In the present disclosure, the “enteric capsule” refers to a capsule that dissolves in a lower part of the small intestine or the large intestine. The enteric capsule can be produced by coating an ordinary gelatin capsule or HPMC capsule with an enteric polymer by a widely used coating procedure using a rotary pan coater, a fluidized bed granulator coater, or the like. Commercially available enteric capsules can be used as well.

Herein, various embodiments of the present disclosure are described, together with certain other embodiments describing their production methods.

In some embodiments of the present pharmaceutical composition for oral administration, the preparation is a granule preparation containing a therapeutic agent coated with an enteric polymer.

In certain other embodiments of the present pharmaceutical composition for oral administration, the preparation is a granule preparation containing a therapeutic agent coated with a layer of colon-targeting polymer.

In yet other embodiments, the pharmaceutical composition for oral administration is a capsule preparation wherein the granule preparation of some embodiments is further encapsulated within an ordinary capsule.

In still other embodiments, the pharmaceutical composition for oral administration is a capsule preparation wherein an uncoated therapeutic agent is encapsulated within an enteric capsule.

In other embodiments, the pharmaceutical composition for oral administration is a capsule preparation wherein the granule preparation of certain embodiment is encapsulated within an enteric capsule.

In still other embodiments, the pharmaceutical composition for oral administration comprises at least one layer of a colon-targeting polymer; in some embodiments the colon-targeting polymer layer is an outer layer.

In still other embodiments, the pharmaceutical composition for oral administration comprises at least one layer of a colon-targeting polymer; in some embodiments the colon-targeting polymer layer is an outer layer; in some embodiments the preparation with at least one layer of a colon-targeting polymer is further encapsulated within an enteric capsule.

In other embodiments, the pharmaceutical composition for oral administration is a tablet preparation wherein the granule preparation of certain embodiments is compression-molded.

In other embodiments, the pharmaceutical composition for oral administration is a tablet preparation wherein a compression-molded product of a therapeutic agent is coated with an enteric polymer.

In other embodiments, the pharmaceutical composition for oral administration is a tablet preparation wherein a compression-molded product of a therapeutic agent is coated with multiple layers comprising one or more polymers, further comprising a colon-targeting polymer layer.

In other embodiments, the pharmaceutical composition for oral administration is a capsule preparation as in other embodiments, further containing an additional, or concomitant, therapeutic agent or drug.

The pharmaceutical composition preparations of the present disclosure can be produced by the following methods.

It should further be appreciated, by the skilled artisan, that one or more of the following Production Method steps, or procedures below, can be repeated multiple times, in a serial-, sequential-, alternating-, and/or iterative-fashion, so as to deposit upon a therapeutic agent surface multiple layers in order to prepare the embodiments described herein.

(Production Method A): The “granule preparation containing a therapeutic agent coated with a colon-targeted polymer” of certain embodiments of the present disclosure can be produced, for example, by the following methods:

A fluidized bed coater (such as a Glatt Mini-coater) can be used for coating. The therapeutic agent to be used is as described above; for example, an activated spherical carbon adsorbent can be used. The colon-targeting polymer to be used, and examples thereof, are as described above. If, in addition, an enteric polymer layer is to be added, triethyl citrate can be used as a plasticizer and talc can be used as a lubricant, in some embodiments. The enteric polymer, the plasticizer, and the lubricant are mixed with a solvent such as ethanol or the like in order to prepare a coating solution. A granule preparation can be obtained by coating a therapeutic agent with the prepared coating solution using the above-described fluidized bed coater.

The amount of the colon-targeting polymer coating can be suitably arranged according to the properties of the colon-targeting polymer so as to serve the function to expose the surface of the therapeutic agent for the first time in the colon. Whether the function to expose the surface of the therapeutic agent for the first time in the colon is served can be verified by the adsorption test described above. As for a range of the amount of coating, in some embodiments, the content of the colon-targeting polymer is about 10 to 100% by weight in terms of polymer solids; in some embodiments, the content of the colon-targeting polymer is about 10 to 60% by weight in terms of polymer solids; and in other embodiments, the content of the colon-targeting polymer is about 20 to 60% by weight in terms of polymer solids, based on the therapeutic agent.

(Production Method B): The capsule preparation of certain embodiments can be produced by encapsulating the granule preparation produced using Production Method A within a gelatin capsule, or an ordinary capsule that uses a cellulose derivative, starch, or the like, according to standard methods, together with one or more additives or the like as necessary. A second therapeutic agent or drug can also be encapsulated within said capsule, in order to provide a combination therapeutic agent or drug.

(Production Method C): The “capsule preparation wherein an uncoated therapeutic agent is encapsulated within a colonic delivery capsule” of certain embodiments can be produced, for example, by the following method:

A non-enteric gelatin capsule or ordinary capsule that uses a cellulose derivative, starch, or the like is coated with the colon-targeting polymer described above using a fluidized bed coater (such as a Glatt Mini-coater) or a rotary pan coater (such as Freund HC-Lab). The amount of the coating can be suitably arranged according to the properties of the colon-targeting polymer so as to serve the function to expose the surface of the therapeutic agent for the first time in the colon. A sub-coating, or first inner layer coating, can also be provided in order to improve the coating suitability of the polymer and reduce the clearance present between the cap and the body of the capsule before coating the capsule with a colon-targeting polymer or optionally, an enteric polymer.

In some embodiments, commercially available enteric capsules can also be used as a substitute to an enteric coating, as long as they serve the function to expose the surface of the therapeutic agent for the first time in the colon.

Whether the colonic capsule of some embodiments, or a layer of colon-targeting polymer of some embodiments, serves the function to expose the surface of the therapeutic agent for the first time in the colon can be verified by, for example, the adsorption test described above or a test as performed in Example 4, 11, or 13 described below).

A small-capsule preparation produced by this method can be further encapsulated within a gelatin capsule or an ordinary capsule that uses a cellulose derivative, starch, or the like together with a further therapeutic agent or drug to form a therapeutic agent combination capsule preparation as well.

(Production Method D): When producing the “capsule preparation wherein the granule preparation of some embodiments is encapsulated within an enteric capsule” of certain other embodiments, the capsule preparation can be produced by combining Production Method A and Production Method C. The amount of the coating on the therapeutic agent is adjusted according to the properties of the capsule. A further therapeutic agent or drug can be further encapsulated to form a therapeutic agent combination or drug combination capsule preparation as well.

(Production Method E): The tablet preparation, wherein the granule preparation of certain embodiments is compression-molded from certain other embodiments, can be obtained by suitably combining the granules obtained by Production Method A with a commonly used excipient, binder, disintegrant, lubricant, and the like, and compression-molding the mixture.

(Production Method F): The tablet preparation wherein a compression-molded product of a therapeutic agent is coated with an enteric polymer or colon-targeting polymer of certain embodiments, can be obtained by suitably combining the therapeutic agent with a commonly used excipient, binder, disintegrant, lubricant, and the like, compression-molding the mixture, and then coating the mixture with an enteric polymer layer or a colon-targeting polymer layer using a widely used tablet coater. A sub-coating, or first inner layer coating, can also be provided in order to improve the coating suitability of the polymer before coating with an enteric polymer layer or colon-targeting polymer layer.

### Compositions of Coatings as % w/w for Excipients

| | | Eudragit | Eudragit | | 90% Zein, 10% |
|---|---|---|---|---|---|
| Type | # | L 30 D | FS 30 D | Polysaccharide | oleic acid |
| pH, | EuPZ-1 | 7 | 13 | 20 | 60.0 |
| Polysaccharide, | EuPZ-2 | 7 | 13 | 40 | 40.0 |
| Protease | EuPZ-3 | 10 | 20 | 30 | 40.0 |
| | EuPZ-4 | 13 | 27 | 20 | 40.0 |
| | EuPZ-5 | 13 | 27 | 40 | 20.0 |
| pH, Protease | EuZ-1 | 7 | 13 | | 80.0 |
| | EuZ-2 | 12 | 23 | | 65.0 |
| | Euz-3 | 17 | 33 | | 50.0 |
| | EuZ-4 | 22 | 43 | | 35.0 |
| Polysaccharide, | PZ-1 | | | 20 | 80.0 |
| Protease | PZ-2 | | | 35 | 65.0 |
| | PZ-3 | | | 50 | 50.0 |
| | PZ-4 | | | 65 | 35.0 |
| | | | NM 30 D | | |
| WI, PSS, Zein | ECPZ-1 | | 30 | 30 | 40.0 |

Kinetics of film dissolution guide an iterative approach to optimize adsorbent release profiles that deliver an adsorbent close to the target colonic site. Zein takes a long time to dissolve under conditions of the colonic environment. In certain embodiments, it is desired that the zein coating should dissolve quickly. The dissolution kinetics of the film is optimized by a variety of techniques, including (1) partial digestion or hydrolysis of a film forming material; (2) incorporation of excipients to enhance dissolution of a film under appropriate conditions; and (3) incorporation of a layer of a swelling agent. The present disclosure provides materials and methods wherein the zein has been partially digested or partially hydrolyzed; neither partial digestion, partial hydrolysis of a film-forming material, nor incorporation of a swelling layer, designed to burst open films, have been previously disclosed or used for delayed/targeted release of an adsorbent material.

One example of partial digestion of the film-forming material is acid/alkali/enzymatic digestion or hydrolysis of zein. The intent is to derive a material or materials that provide a barrier to film dissolution until the targeted colonic region is reached, then break down quickly at a desired target site. Using procedures listed herein (film formation and dissolution studies), the present disclosure provides materials and methods to optimize both a targeted release distribution profile as well as the kinetics of said targeted release. While zein is an example, polymer-length modification is evaluated and utilized with other film-formers.

The present disclosure provides to the skilled artisan, materials and methods that are broadly applicable to a wide variety of molecules one would desire to administer alone, or to co-administer, along with other therapeutic agents, for targeted release in the colon, particularly the descending colon.

### Example 5

### Development and Assessment of Microbial-Responsive Layers

### Coating Development Process:

### Strategy #1: Insoluble Polymeric Coatings

Microbial-sensitive layers or coatings based on insoluble polymeric dispersions with or without plasticizers were developed and tested. Insoluble polymeric dispersions included aqueous ethyl cellulose dispersions (e.g., Surelease® and Aquacoat® ECD 30, Colorcon, Inc., Harleysville, PA), and poly(ethyl acrylate-co-methyl methacrylate) co-polymers (e.g., Eudragit® NM 30 D, Evonik Industries AG, Darmstadt, DE). These insoluble polymeric dispersions were strengthened using various amounts of plasticizers (e.g., PlasACRYL®, Evonik Industries AG, Darmstadt, DE; dibutyl sebacate (DBS), triethyl citrate (TEC)) with heating/acidification according to standard methods. The maximum plasticizer equivalent to the strongest coating was determined for each polymeric dispersion type.

In order to produce layers for colonic targeting, various microbial-responsive components were incorporated into the insoluble polymeric dispersions and tested. These microbial-responsive components included high amylose corn starch (Roquette® Amylo N-400 Starch, Knowde, Inc., San Jose, CA), soluble pea protein (Roquette® NUTRALYS® S, Knowde, Inc., San Jose, CA), and soluble fiber (Roquette® Nutriose, Knowde, Inc., San Jose, CA). Sodium octenyl succinate starch (SOS) (e.g., Roquette® CLEARGUM®, Knowde, Inc., San Jose, CA), and fructo-oligosaccharides (FOS) are also useful as microbial-responsive components.

### Strategy #2: Protein Coatings

Zein, a corn protein, is substantially resistant to digestion by the human enzymes in the upper gastrointestinal tract. A solution of 15% zein and 85% hydroalcohol was tested as a base for microbial-sensitive layers or coatings (FloZein™ or AquaZein™, FloZein Products, Ashburnham, MA).

Pectin, a highly water soluble molecule, can also be used. The absorption of water by pectin leads to swelling and disruption of the coating. To control the rate or timing of disruption, pectin can be modified to reduce solubility and/or insoluble polymer dispersions can be incorporated into the coating.

### Overview of Coating Process:

The coating process consisted of the following steps. First, the various components were mixed to generate a coating formulation. Next, therapeutic agent core particles (in these experiments, activated carbon particles were used) were coated with the formulation using a fluidized bed dryer (Mini-Glatt, Glatt GmbH, Blinzen, DE). If the coating process was successful, determined using qualitative measures (e.g., visual inspection methods described in Example 4), p-cresol binding studies were performed in simulated upper GI fluid. If the coating performed well, a reciprocating cylinder (USP Apparatus 3) was used to test the coating to at 6, 8 and 12 hours. If the coating met the requirements of (1) low binding % to p-cresol and (2)<10% uncoated binding in simulated fluids after 5-6 hours, it was further tested in an in vitro model for the combined simulation of the physiological, chemical and microbiological properties of the gastrointestinal tract (i.e., Simulator of the Human Intestinal Microbial Ecosystem or SHIME® assay, ProDigest, Ghent, BE).

### Results:

To model degradation of microbial-responsive coatings in the upper GI, therapeutic agent core particles were coated with different microbial-responsive coatings and exposed to phosphate buffered saline (PBS)+p-cresol to develop p-cresol binding profiles. In this experiment, low levels of p-cresol binding are desirable and indicate that the coating is substantially intact and preventing binding to the therapeutic agent core.

To model degradation of microbial-responsive coatings in the colon, therapeutic agent core particles were coated with different microbial-responsive coatings and the SHIME assay was used to develop p-cresol and indole binding profiles. In this experiment, substantial binding of p-cresol/indole is desirable and indicates that the microbial-responsive coating has degraded and exposed the therapeutic agent core and allowed binding of the metabolite (e.g., p-cresol) to the therapeutic agent.

### Results for 100% Surelease Coating:

TABLE 20

| Time, | 7% | 12% | 18% | 24% | 29% |
|---|---|---|---|---|---|
| h | Average Amount of p-cresol Adsorbed by Coated Particles (μg) | | | | |
| 1 | 107 | 67 | −17 | −76 | −48 |
| 4 | 735 | 687 | 207 | 132 | 73 |
| 24 | 2075 | 1758 | 1214 | 779 | 694 |
| 48 | 2138 | 1894 | 1485 | 1034 | 775 |

TABLE 21

| Time, | 7% | 12% | 18% | 24% | 29% |
|---|---|---|---|---|---|
| h | % Total p-cresol Binding | | | | |
| 1 | 3 | 2 | 0 | −2 | −1 |
| 4 | 20 | 18 | 6 | 4 | 2 |
| 24 | 58 | 49 | 34 | 22 | 19 |
| 48 | 60 | 53 | 42 | 29 | 22 |

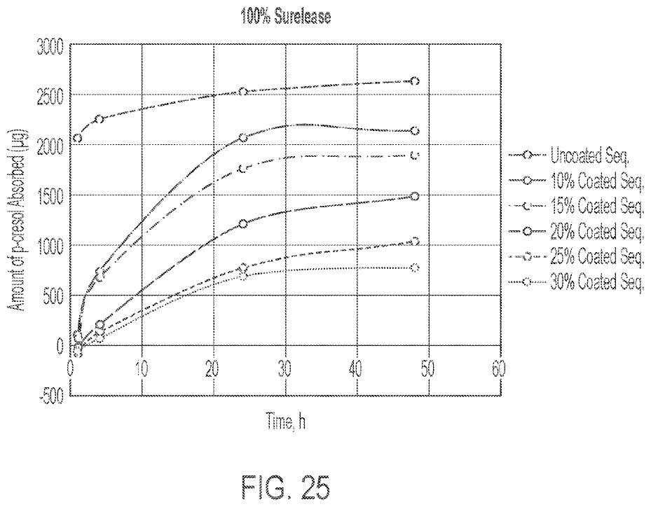

### Also see FIG. 25

### Results for 20% High Amylo N-400+Surelease:

TABLE 22

| | Amount (μg) p-cresol Absorbed by Coated Particles | | | | |
|---|---|---|---|---|---|
| Time, | % Coating | | | | |
| h | 9% | 15% | 20% | 24% | E.O.C % |
| 1 | 365 | 212 | 163 | 117 | 95 |
| 3 | 931 | 498 | 369 | 272 | 196 |
| 68 | 2858 | 2834 | 2734 | 2592 | |

TABLE 23

| Time, | 9% | 15% | 20% | 24% | E.O.C % |
|---|---|---|---|---|---|
| h | % Total p-cresol Binding | | | | |
| 1 | 9.6 | 5.6 | 4.3 | 3.1 | 2.5 |
| 3 | 24.6 | 13.1 | 9.7 | 7.2 | 5.2 |
| 68 | 76.0 | 75.4 | 72.7 | 69.0 | N/A |

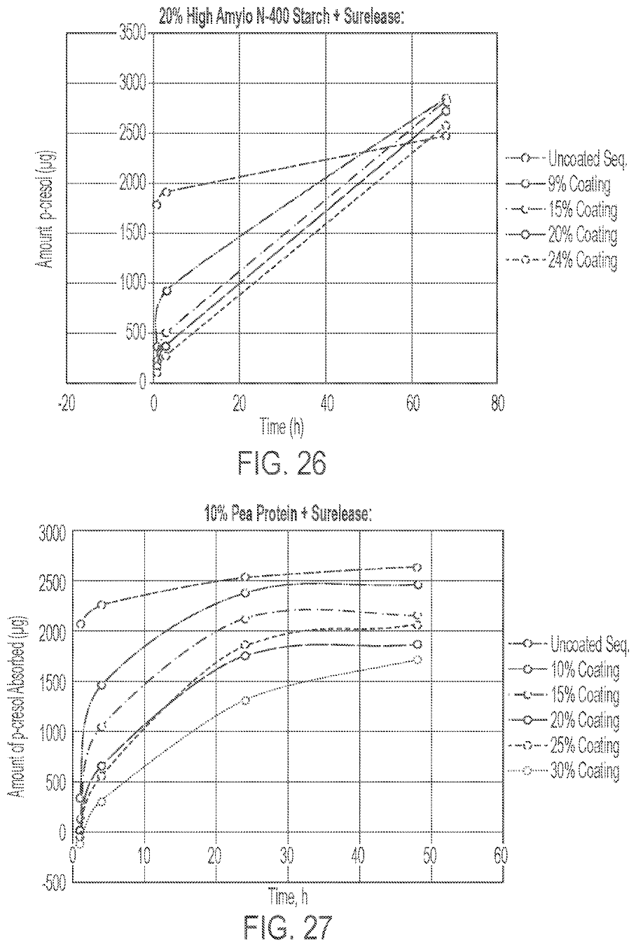

See also FIG. 26

### Results for 10% Pea Protein+Surelease:

TABLE 24

| Time, | 10% | 15% | 20% | 25% | 30% |
|---|---|---|---|---|---|
| h | Average Amount of p-cresol Absorbed by Coated Particles (μg) | | | | |
| 1 | 337 | 115 | 11 | −46 | −130 |
| 4 | 1454 | 1035 | 645 | 552 | 300 |
| 24 | 2374 | 2117 | 1745 | 1848 | 1300 |
| 48 | 2458 | 2150 | 1861 | 2051 | 1703 |

TABLE 25

| Time, | 10% | 15% | 20% | 25% | 30% |
|---|---|---|---|---|---|
| h | % Total p-cresol Binding | | | | |
| 1 | 9 | 3 | 0 | −1 | −4 |
| 4 | 39 | 28 | 17 | 15 | 8 |
| 24 | 77 | 59 | 49 | 52 | 36 |
| 48 | 69 | 61 | 52 | 58 | 48 |

See also FIG. 27

### Results for 10% Nutriose+Surelease:

TABLE 26

| Time, | 10% | 15% | 20% | 25% |
|---|---|---|---|---|
| h | Avg Amount of p-cresol Absorbed by Coated Particles (μg) | | | |
| 1 | 166 | 13 | −21 | −75 |
| 4 | 1274 | 841 | 585 | 350 |
| 24 | 2391 | 2139 | 1608 | 1302 |
| 48 | 2511 | 2274 | 2006 | 1571 |

TABLE 27

| | | | | |
|---|---|---|---|---|
| Time, | 10% | 15% | 20% | 25% |

| | |
|---|---|
| h | % Total p-cresol Binding |

| | | | | |
|---|---|---|---|---|
| 1 | 5 | 0 | −1 | −2 |
| 4 | 34 | 23 | 16 | 9 |
| 24 | 67 | 60 | 45 | 37 |
| 48 | 71 | 64 | 56 | 44 |

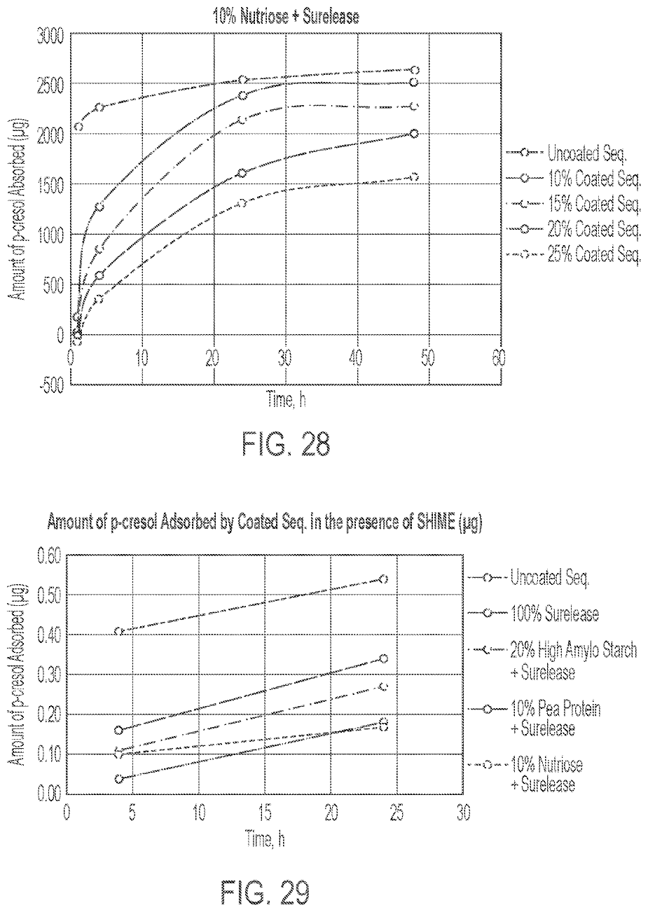

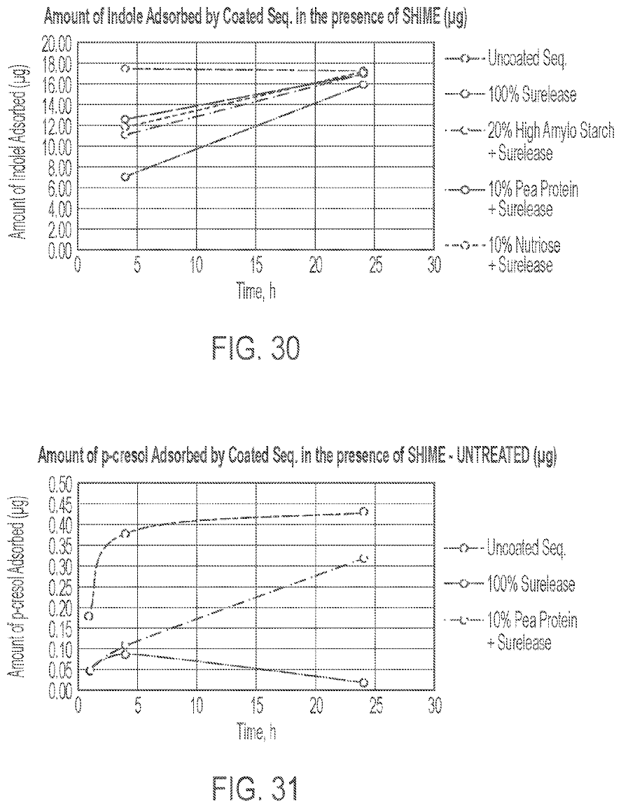

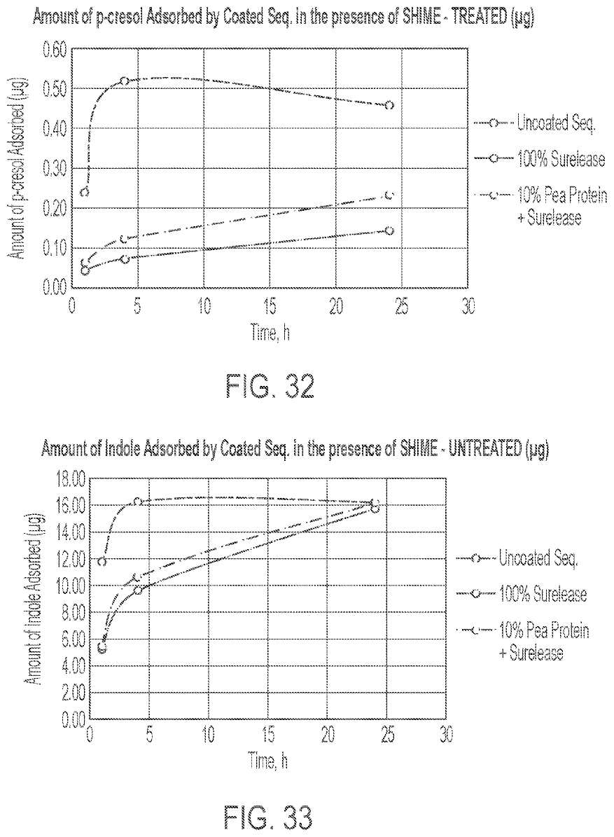

See also FIG. 28

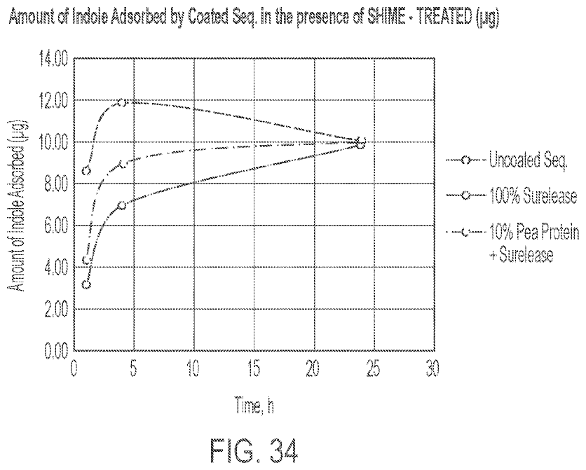

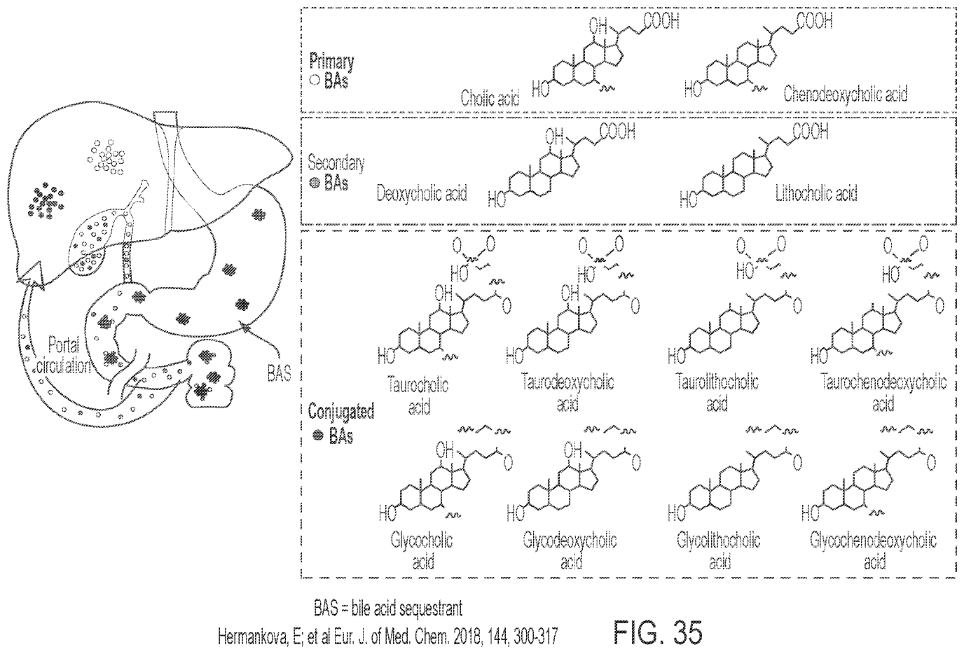

FIG. 29—FIG. 34 show data from SHIME assays for adsorption of different metabolites using activated carbon particles as the therapeutic agent core.

### Example 6

### Selective Sequestration of Secondary Bile Acids Using Gut Restricted Synthetic Polymers

Bacterial gut microbes in the small intestine and colon metabolize conjugated primary bile acids to produce unconjugated primary and secondary bile acids. Increased levels of secondary bile acids in the colon can increase the risk of colon damage including inflammation, increased gastrointestinal permeability, and cancer. Increased levels of secondary bile acids in the liver can increase the risk of liver damage including inflammation, NASH, fibrosis, cirrhosis, and cancer. Elevated secondary bile acids can cause inflammatory and immunological responses that extend beyond the gut to the peripheral and the central nervous system. A cationic polymer molecule that selectively binds secondary bile acids like DCA and LCA and their conjugated forms in the GI could prevent colon damage, the reabsorption of secondary bile acids to the liver, and inflammatory and immunological sequelae.

A therapeutic with this profile could be used treat diseases like colon cancer, Crohn's Disease, other inflammatory bowel diseases, NASH, fibrosis, cirrhosis, HCC, Parkinson's disease, Alzheimer's disease and autoimmune diseases as well as modulate immune responses. A targeted approach, in which synthetic polymers selectively bind secondary bile acids over primary bile acids in the colon, can reduce or avoid upregulation of bile acid synthesis as has been seen with unselective bile acid sequestrants. A compound of this type that does not bind other acidic metabolites would also have an advantage over unselective binders. An additional advantage of using a polymeric bile acid sequestrant is the lack of systemic exposure which should provide a favorable safety profile.

### Methods

An assay was developed that was capable of determining the binding capacity of synthetic polymers with cationic groups such as those derived from chitosan (WO2013006458; US20140080785). A mixture of bile acid and several short chain fatty acids in assay buffer were mixed with the polymer. Samples were removed at specific time points and passed through a molecular weight cut-off filter which removed polymer and anything that was bound to it. The concentration of bile acid remaining in solution was then determined using LC-MS/MS.

### Assay for Measuring Bile Acid Binding Capacity of Cationic Polymeric Molecules; Bile Acid Binding Study Procedure

### Procedure for the Phosphate Buffer

For preparation of 5× buffer, weigh:1.2 g of monobasic sodium phosphate, 1.6 g of dibasic sodium phosphate, 5 g of sodium chloride, 3.3 g of sodium acetate, 1.9 g of sodium propionate and 2.2 g of sodium butyrate.

To a 500 mL beaker, add 200 mL of purified water using a graduated cylinder and add the six weighed chemicals.

Place a stir bar and allow it to stir on a stir plate until the chemicals are completely dissolved.

Measure the pH of this mixture with pH meter and pH probe. Dropwise, with stirring add Sodium Hydroxide, (5 N) to adjust the pH to 7.2.

When pH 7.2 is obtained, add the mixture to a graduated cylinder and add purified water to 1000 mL final volume of the 5× buffer.

### Diluent

Add 60 mL 5× buffer into 240 mL water and mix well.

### Prepare Primary Solutions of Each Bile Acids

The following bile acids were used in the study: Cholic acid (CA), deoxycholic acid (DCA), taurocholic acid (TCA), taurodeoxycholic acid (TDCA), glycocholic acid (GCA), glycodeoxycholic acid (GDCA), chenodeoxycholic acid (CDCA), glycochenodeoxycholic acid (GCDCA) and taurochenodeoxycholic acid (TCDCA).

15 mg of each bile acid was weighed and 50 mL of diluent was added to achieve the concentration of 0.30 mg/ml. The mixtures were stored at −20° C. before use. CA and DCA did not completely dissolve in buffer. The solutions were stirred at RT overnight, and the solutions were filtered with a 0.45 m filter before use. The filtered solutions were used to determine the 0 hour concentration.

### Procedure for Binding Test

For AB-2106-02, Weigh 2 mg of adsorbent into a 4 mL vial, add 2 mL of bile acid primary solution into the vial and mix well, separate the sample solution into 4 vials with 0.5 mL/vial. Repeat to generate triplicate samples for each adsorbent. Label with adsorbent name, replicate—1, 2 or 3. (Resin: 1 mg/mL)

For the other resins, weigh about 0.5 mg of adsorbent into 4 of 4 mL vials, add 0.5 mL of bile acid primary solution into each vial and mix well. Repeat to generate triplicate samples for each adsorbent. Label with adsorbent name, replicate—1, 2 or 3. (Resin: 1 mg/mL) Note the starting time. Screw the cap and place the tubes in incubator-shaker at 37° C. and 60 revolutions.

Pull samples at 1, 3, 6 and 24 hours. Filter the samples through a molecular weight cut-off filter (Amicon® Ultra, 0.5 mL, 3K NMWL, pre-saturate the filters with 0.5 mL bile acids, 14000 rpm, 20 minutes).

Quantitate the filtrate by LC-MS/MS.

TABLE 28

| Column | Ascentis Express C18, 2.7μ*4.6*100 mm | | | | | |
|---|---|---|---|---|---|---|
| Mobile Phase | MPA: 5 mM ammonium acetate and 0.012% FA in MeOH | | | | | |
| | MPA: 5 mM ammonium acetate and 0.012% FA in water | | | | | |
| | | % | % | | | |
| | Time | Mobile | Mobile | | | |
| | (min) | Phase A | Phase B | | | |
| Gradient | 0 | 80 | 20 | | | |
| | 1 | 80 | 20 | | | |
| | 3 | 30 | 70 | | | |
| | 6 | 10 | 90 | | | |
| | 7 | 10 | 90 | | | |
| | 8 | 80 | 20 | | | |
| | 10 | 80 | 20 | | | |
| MS switching | 0 | to waste | | | | |
| valve (the second | 3 min | to ms | | | | |
| method) | | | | | | |
| Flow rate | 1 ml/min | | | | | |
| Injection Volume | 1 μl | | | | | |
| Column | 30° C. | | | | | |
| | Polarity | Negative | | | | |
| | Compound | Precursor | Product | | | Collision |
| | Name | Ion | Ion | Dwell | Fragment | Energy |
| MS | TCA | 514.3 | 124 | 200 | 220 | 50 |
| | TDCA | 498.3 | 124 | 200 | 220 | 50 |
| | CA | 407.2 | 343.2 | 200 | 220 | 33 |
| | DCA | 391.2 | 345.2 | 200 | 300 | 36 |

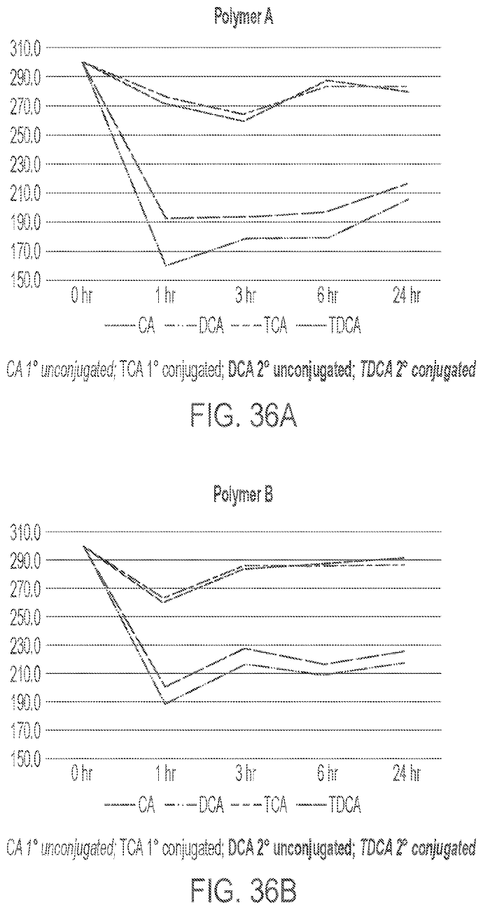

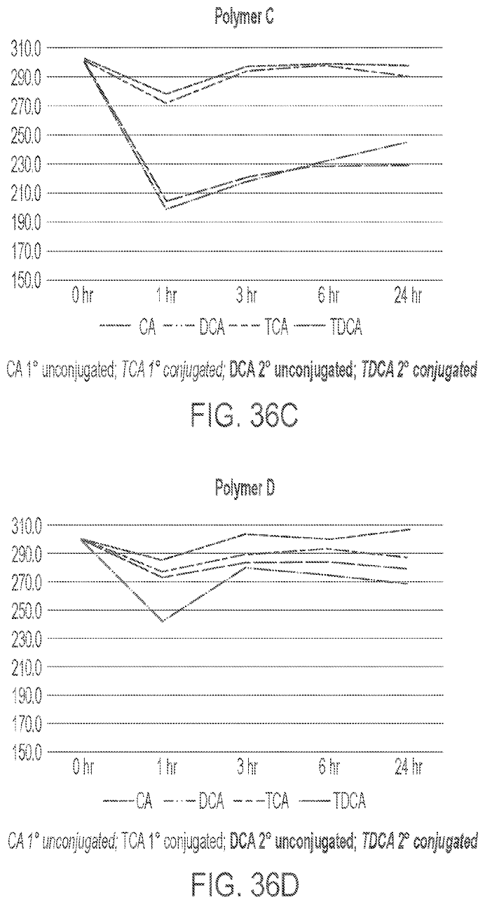

Typical results for a series of semi-synthetic glucosamine based cationic polymers are shown in FIG. 36A-D, in which the concentration of bile acid remaining in solution (g/mL) was plotted vs 4 time points out to 24 hours. A clear SAR was demonstrated, and Polymer A, Polymer B and Polymer C showed a preference for binding secondary conjugated and unconjugated bile acids (DCA, TDCA) over primary conjugated and unconjugated bile acids (CA, TCA). Polymer D showed only weak binding capacity of the tested bile acids and little to no selectivity.

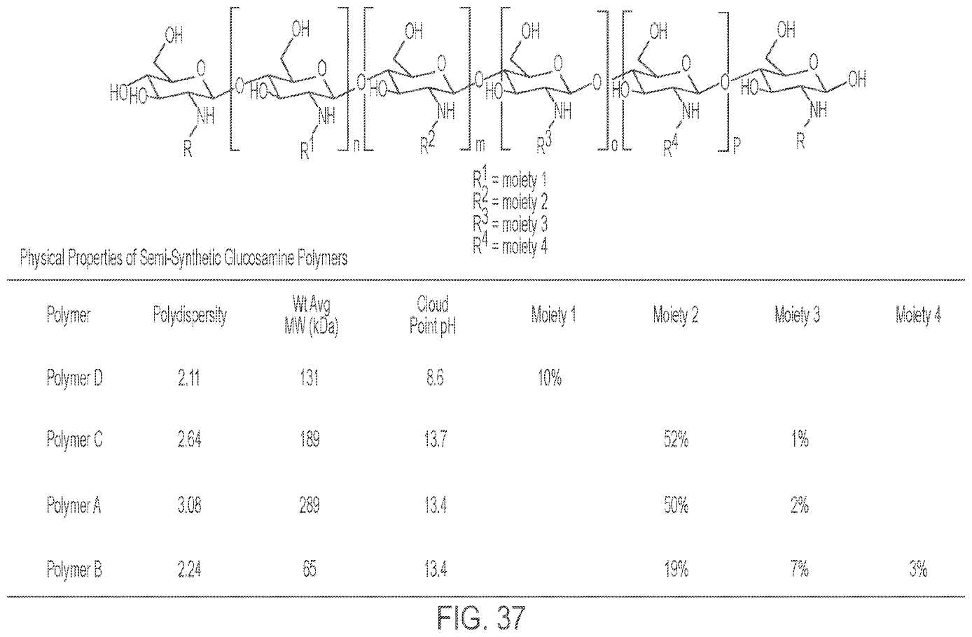

The primary difference between Polymer D and the other polymers was the nature of the cationic R group, moiety 1 for Polymer D, and moiety 2 for the other polymers. Polymer A, Polymer B and Polymer C all contained the same moiety but at varying percentages. Polymer A, Polymer B and Polymer C each contained an additional moiety. In the case of Polymer B two additional moieties were incorporated into the polymer. Other properties such as polydispersity, wt avg MW and cloud point are described in FIG. 37.

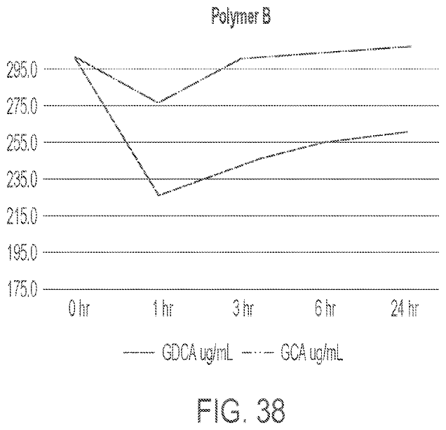

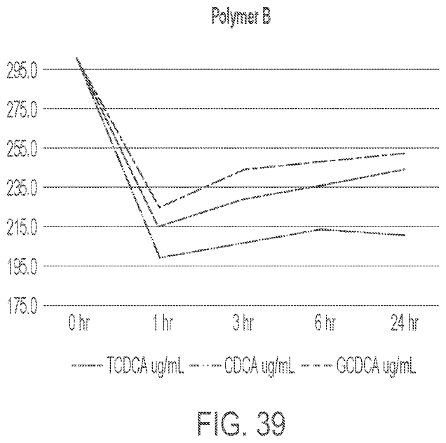

In order to generate additional structure-activity relationship (SAR) data, the assay was expanded to measure the binding affinity of Polymer B vs other bile acids GDCA, GCA, CDCA, GCDCA, TCDCA. Once again, Polymer B showed similar preference for the conjugated secondary bile acid GDCA vs primary conjugated bile acid GCA (FIG. 38). Polymer B also showed strong binding affinity to CDCA as well as its glycol and tauro conjugated forms (FIG. 39). The substituents described above and other substituents could be added to other polymeric backbones to obtain bile acids sequestrants with a range of specificity and bile acid binding affinities.

Cholestyramine, colesevelam and colestipol are FDA approved polycationic resins which are capable of sequestering hydrophobic acidic compounds including bile acids. (See, e.g., (Hermankova et al. (2018), Eur. J. of Med. Chem. 144:300-317.) Several disadvantages associated with the therapeutic use of unselective polycationic resins are high required doses, the removal of all bile acids resulting in the upregulation of bile acid synthesis, and binding of other anionic substances, which could lead to undesired off-target side effects.

The chitosan polymers described herein showed an unexpected preference for secondary over primary bile acids. A molecule that selectively binds secondary hydrophobic bile acids in the colon can prevent colon damage and the reabsorption of secondary bile acids to the liver and can therefore be used as a therapeutic for the treatment of disease. Selectively binding to secondary bile acids can provide the advantage of not causing up-regulation of bile acid synthesis. The fact that these polymers are not systemically available can result in a favorable safety/efficacy profile.

Compounds of this class are dosed in vivo to determine the effect of selective secondary bile acid binders on the ratio of bile acids in the plasma, cecum and feces. Several animal models of colon cancer and HCC/NASH exist, and the effect of selective secondary bile acid binders on disease severity and progression are measured.

Based on the binding affinity of capped cyclodextrins for secondary bile acids, R1 groups from the most active capped cyclodextrins are incorporated into glucosamine polymers to target adsorption of secondary bile acids and generate unique semi-synthetic polymers with glucosamine backbones.

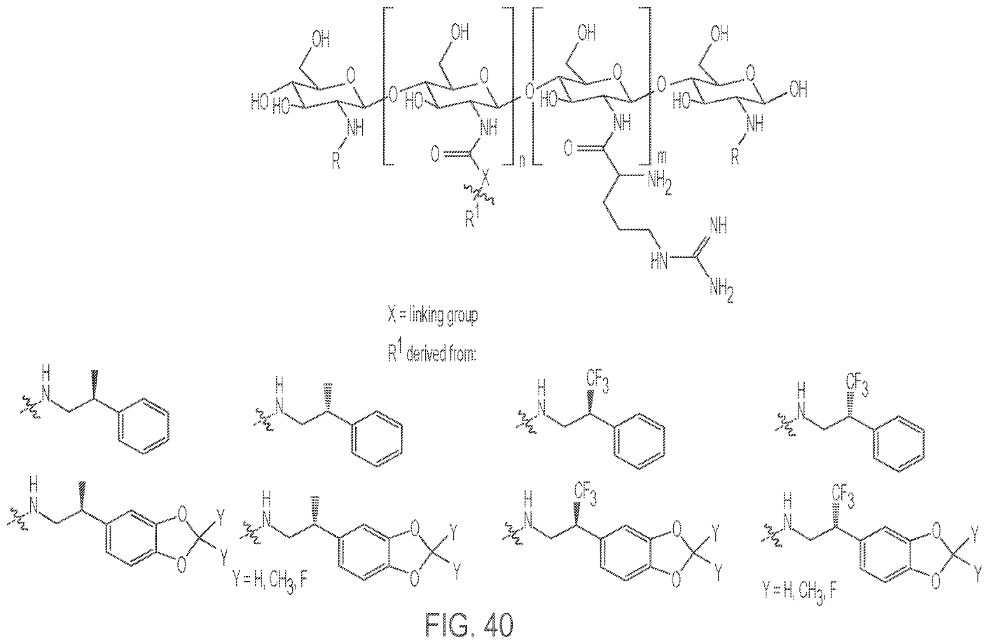

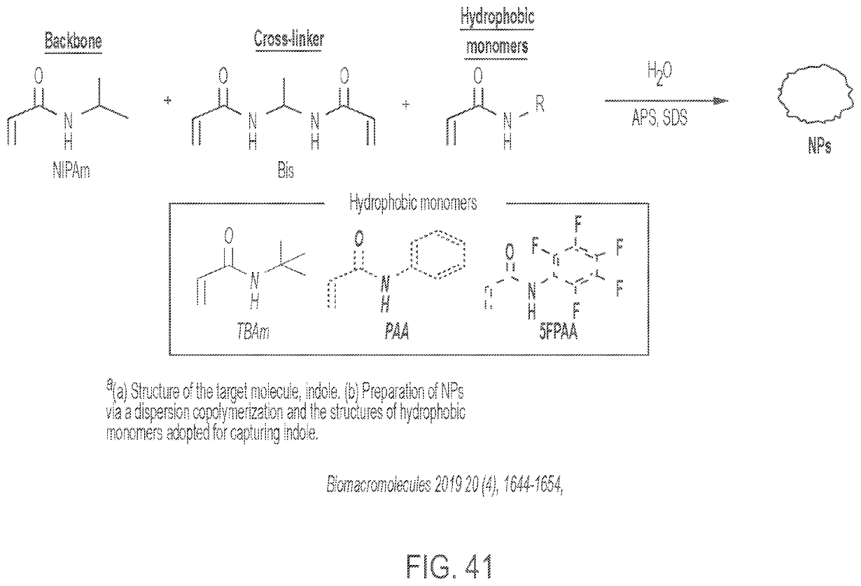

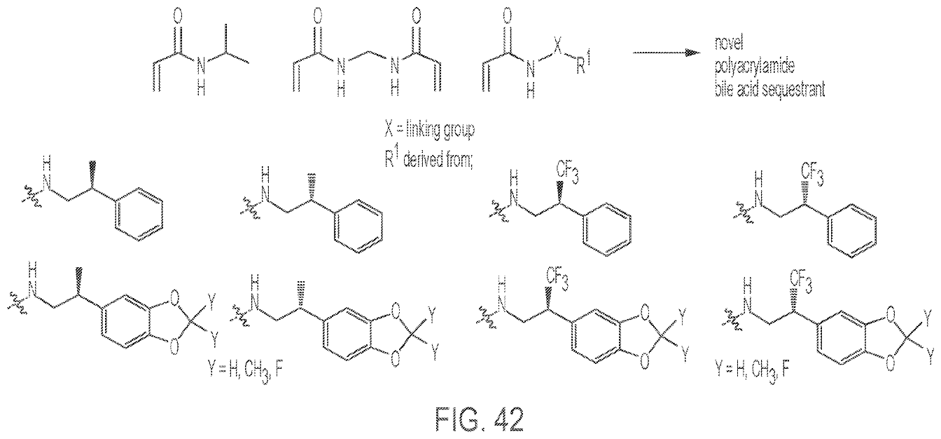

Another synthetic polymer system that binds small molecules was described by Okishima et al. (2019), Biomacromolecules 20(4):1644-1654.. In this polymer system, the polymer backbone was based on acrylamides. The binding of these polymers are targeted to secondary bile acids by incorporating R1 groups from the most active capped cyclodextrins in place of the hydrophobic monomers. This generates unique synthetic polymers with acrylamide backbones that are targeted to binding secondary bile acids. Previous technology has focused on producing nanoparticles with this technology to make the polymers more bioavailable. In this study, the goal is to make micro particles to prevent systemic exposure. See FIG. 41 and FIG. 42.

1.-18. (canceled)

19. A pharmaceutical composition for oral administration comprising:
a therapeutic agent core comprising porous activated carbon particles having substantially spherical particles that have a minimum average particle diameter of at least 0.005 mm and a maximum average particle diameter of less than 1.5 mm; and
a plurality of degradable layers for exposing a surface of the therapeutic agent core, wherein there is an inner degradable layer and an outer degradable layer;
wherein the outer layer comprises an acrylic based polymer selected from the group consisting of (a) methacrylic acid copolymer S or (b) a co-polymer of methacrylic acid and methyl methacrylate, and further wherein the outer layer comprises between about 20-40% weight of the acrylic polymer based on the therapeutic core, and
wherein the inner layer comprises a polymer selected from the group consisting of (a) ethyl cellulose or (b) poly(ethyl acrylate co-methyl methacrylate) co-polymer.

20.-26. (canceled)

27. The pharmaceutical composition of claim 19 wherein the inner layer further comprises a microbial responsive component selected from the group consisting of high amylase corn starch, pea protein, soluble fiber, sodium octenyl succinate starch and fructo-oligosaccharides.

28. The pharmaceutical composition of claim 27 wherein the microbial responsive component is pea protein.

29. The pharmaceutical composition of claim 19 wherein the inner layer and/or the outer layer comprises an anti-tacking agent.

30. The pharmaceutical composition of claim 19 wherein the inner layer and/or the outer layer comprises a plasticizer.

31. The pharmaceutical composition of claim 27 wherein the inner layer and/or the outer layer comprises an anti-tacking agent.

32. The pharmaceutical composition of claim 27 wherein the inner layer and/or the outer layer comprises a plasticizer.

33. The pharmaceutical composition of claim 28 wherein the inner layer and/or the outer layer comprises an anti-tacking agent.

34. The pharmaceutical composition of claim 28 wherein the inner layer and/or the outer layer comprises a plasticizer.
